SEQUENCE LISTING (205 sequences)

SEQ ID NO: 1 - protein, 119 aa, Artificial sequence
  an artificially synthesized sequence
QVQLVESGGGVVQPGGSLRLSCAASGFTFSSYTMSWVRQAPGQGLEWMGTISSGGGRTYYPDSVKGRFTISRDNSKNTLYLQMNSLRDEDTAVYYCARGDYRYDGFAYWGEGTLVTVSS

SEQ ID NO: 2 - protein, 119 aa, Artificial sequence
  an artificially synthesized sequence
QVQLVESGGGVVQPGGSLRLSCAASGFTFSSYTMSWVREAPGQGLEWMGTISSGGGRTYYPDSVKGRFTISRDNSKNTLYLQMNSLRDEDTAVYYCARGDYRYDGFAYWGEGTLVTVSS

SEQ ID NO: 3 - protein, 128 aa, Artificial sequence
  an artificially synthesized sequence
QVQLVESGGGLVQPGRSLRLSCAASGFKFSNVWFHWVRKAPGKGLEWVAQIKDYYNAYAAYYAPSVKGRFTISRDDSKNSIYLQMNSLKTEDTAVYYCHYVHYASASTLLPAEGVDAWGQGTTVTVSS

SEQ ID NO: 4 - protein, 128 aa, Artificial sequence
  an artificially synthesized sequence
QVQLVESGGGLVQPGRSLRLSCAASGFKFSNVWFHWVRKAPGKGLEWVAQIKDYYNAYAGYYHPSVKGRFTISRDDSKNSIYLQMNSLKTEDTAVYYCHYVHYAAASQLLPAEGVDAWGQGTTVTVSS

SEQ ID NO: 5 - protein, 128 aa, Artificial sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSS

SEQ ID NO: 6 - protein, 128 aa, Artificial sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSS

SEQ ID NO: 7 - protein, 5 aa, Artificial sequence
  an artificially synthesized sequence
SYTMS

SEQ ID NO: 8 - protein, 5 aa, Artificial sequence
  an artificially synthesized sequence
SYTMS

SEQ ID NO: 9 - protein, 5 aa, Artificial sequence
  an artificially synthesized sequence
NVWFH

SEQ ID NO: 10 - protein, 5 aa, Artificial sequence
  an artificially synthesized sequence
NVWFH

SEQ ID NO: 11 - protein, 5 aa, Artificial sequence
  an artificially synthesized sequence
NVWFH

SEQ ID NO: 12 - protein, 5 aa, Artificial sequence
  an artificially synthesized sequence
NVWFH

SEQ ID NO: 13 - protein, 17 aa, Artificial sequence
  an artificially synthesized sequence
TISSGGGRTYYPDSVKG

SEQ ID NO: 14 - protein, 17 aa, Artificial sequence
  an artificially synthesized sequence
TISSGGGRTYYPDSVKG

SEQ ID NO: 15 - protein, 19 aa, Artificial sequence
  an artificially synthesized sequence
QIKDYYNAYAAYYAPSVKG

SEQ ID NO: 16 - protein, 19 aa, Artificial sequence
  an artificially synthesized sequence
QIKDYYNAYAGYYHPSVKG

SEQ ID NO: 17 - protein, 19 aa, Artificial sequence
  an artificially synthesized sequence
QIKDYYNAYAAYYAPSVKG

SEQ ID NO: 18 - protein, 19 aa, Artificial sequence
  an artificially synthesized sequence
QIKDYYNAYAGYYHPSVKG

SEQ ID NO: 19 - protein, 10 aa, Artificial sequence
  an artificially synthesized sequence
GDYRYDGFAY

SEQ ID NO: 20 - protein, 10 aa, Artificial sequence
  an artificially synthesized sequence
GDYRYDGFAY

SEQ ID NO: 21 - protein, 17 aa, Artificial sequence
  an artificially synthesized sequence
VHYASASTLLPAEGVDA

SEQ ID NO: 22 - protein, 17 aa, Artificial sequence
  an artificially synthesized sequence
VHYAAASQLLPAEGVDA

SEQ ID NO: 23 - protein, 17 aa, Artificial sequence
  an artificially synthesized sequence
VHYASASTLLPAEGVDA

SEQ ID NO: 24 - protein, 17 aa, Artificial sequence
  an artificially synthesized sequence
VHYAAASQLLPAEGVDA

SEQ ID NO: 25 - protein, 107 aa, Artificial sequence
  an artificially synthesized sequence
DIQMTQSPSSLSASVGDRVTITCRASYNIDSYLAWYQQKPGKAPELLIYYSTLLVDGVPDRFSGSRSGTDFTLTISSLQPEDFATYYCQHYYSIPYTFGQGTKVEIE

SEQ ID NO: 26 - protein, 107 aa, Artificial sequence
  an artificially synthesized sequence
DIQMTQSPSSLSASVGDRVTITCRASYNIDSYLAWYQKKPGKAPELLIYYSTLLVDGVPDRFSGSRSGTDFTLTISSLQPEDFATYYCQHYYSIPYTFGQGTKVEIE

SEQ ID NO: 27 - protein, 112 aa, Artificial sequence
  an artificially synthesized sequence
DIVMTQSPLSLPVTPGEPASISCQPSQEVVHMNRNTYLHWYQEKPGQAPRLLIYKVSNRFPGVPDRFSGSGSGTDFTLKISRVEAEDVGVYYCAQGTSHPFTFGQGTKLEIK

SEQ ID NO: 28 - protein, 112 aa, Artificial sequence
  an artificially synthesized sequence
DIVMTQSPLSLPVTPGEPASISCQPSQEVVHMNRNTYLHWYQQKPGQAPRLLIYKVSNRFPGVPDRFSGSGSGTDFTLKISRVEAEDVGVYYCAQGTSHPFTFGQGTKLEIK

SEQ ID NO: 29 - protein, 11 aa, Artificial sequence
  an artificially synthesized sequence
RASYNIDSYLA

SEQ ID NO: 30 - protein, 11 aa, Artificial sequence
  an artificially synthesized sequence
RASYNIDSYLA

SEQ ID NO: 31 - protein, 16 aa, Artificial sequence
  an artificially synthesized sequence
QPSQEVVHMNRNTYLH

SEQ ID NO: 32 - protein, 16 aa, Artificial sequence
  an artificially synthesized sequence
QPSQEVVHMNRNTYLH

SEQ ID NO: 33 - protein, 7 aa, Artificial sequence
  an artificially synthesized sequence
YSTLLVD

SEQ ID NO: 34 - protein, 7 aa, Artificial sequence
  an artificially synthesized sequence
YSTLLVD

SEQ ID NO: 35 - protein, 7 aa, Artificial sequence
  an artificially synthesized sequence
KVSNRFP

SEQ ID NO: 36 - protein, 7 aa, Artificial sequence
  an artificially synthesized sequence
KVSNRFP

SEQ ID NO: 37 - protein, 9 aa, Artificial sequence
  an artificially synthesized sequence
QHYYSIPYT

SEQ ID NO: 38 - protein, 9 aa, Artificial sequence
  an artificially synthesized sequence
QHYYSIPYT

SEQ ID NO: 39 - protein, 9 aa, Artificial sequence
  an artificially synthesized sequence
AQGTSHPFT

SEQ ID NO: 40 - protein, 9 aa, Artificial sequence
  an artificially synthesized sequence
AQGTSHPFT

SEQ ID NO: 41 - protein, 447 aa, Artificial sequence
  an artificially synthesized sequence
QVQLVESGGGVVQPGGSLRLSCAASGFTFSSYTMSWVREAPGQGLEWMGTISSGGGRTYYPDSVKGRFTISRDNSKNTLYLQMNSLRDEDTAVYYCARGDYRYDGFAYWGEGTLVTVSSASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 42 - protein, 447 aa, Artificial sequence
  an artificially synthesized sequence
QVQLVESGGGVVQPGGSLRLSCAASGFTFSSYTMSWVRQAPGQGLEWMGTISSGGGRTYYPDSVKGRFTISRDNSKNTLYLQMNSLRDEDTAVYYCARGDYRYDGFAYWGEGTLVTVSSASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 43 - protein, 437 aa, Artificial sequence
  an artificially synthesized sequence
DIQMTQSPSSLSASVGDRVTITCRASYNIDSYLAWYQQKPGKAPELLIYYSTLLVDGVPDRFSGSRSGTDFTLTISSLQPEDFATYYCQHYYSIPYTFGQGTKVEIESSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 44 - protein, 437 aa, Artificial sequence
  an artificially synthesized sequence
DIQMTQSPSSLSASVGDRVTITCRASYNIDSYLAWYQQKPGKAPELLIYYSTLLVDGVPDRFSGSRSGTDFTLTISSLQPEDFATYYCQHYYSIPYTFGQGTKVEIESSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 45 - protein, 447 aa, Artificial sequence
  an artificially synthesized sequence
QVQLVESGGGVVQPGGSLRLSCAASGFTFSSYTMSWVREAPGQGLEWMGTISSGGGRTYYPDSVKGRFTISRDNSKNTLYLQMNSLRDEDTAVYYCARGDYRYDGFAYWGEGTLVTVSSASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTREELSLSP

SEQ ID NO: 46 - protein, 447 aa, Artificial sequence
  an artificially synthesized sequence
QVQLVESGGGVVQPGGSLRLSCAASGFTFSSYTMSWVRQAPGQGLEWMGTISSGGGRTYYPDSVKGRFTISRDNSKNTLYLQMNSLRDEDTAVYYCARGDYRYDGFAYWGEGTLVTVSSASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTREELSLSP

SEQ ID NO: 47 - protein, 437 aa, Artificial sequence
  an artificially synthesized sequence
DIQMTQSPSSLSASVGDRVTITCRASYNIDSYLAWYQQKPGKAPELLIYYSTLLVDGVPDRFSGSRSGTDFTLTISSLQPEDFATYYCQHYYSIPYTFGQGTKVEIESSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 48 - protein, 447 aa, Artificial sequence
  an artificially synthesized sequence
QVQLVESGGGVVQPGGSLRLSCAASGFTFSSYTMSWVRQAPGQGLEWMGTISSGGGRTYYPDSVKGRFTISRDNSKNTLYLQMNSLRDEDTAVYYCARGDYRYDGFAYWGEGTLVTVSSASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 49 - protein, 447 aa, Artificial sequence
  an artificially synthesized sequence
QVQLVESGGGVVQPGGSLRLSCAASGFTFSSYTMSWVRQAPGQGLEWMGTISSGGGRTYYPDSVKGRFTISRDNSKNTLYLQMNSLRDEDTAVYYCARGDYRYDGFAYWGEGTLVTVSSASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTREELSLSP

SEQ ID NO: 50 - protein, 214 aa, Artificial sequence
  an artificially synthesized sequence
DIQMTQSPSSLSASVGDRVTITCRASYNIDSYLAWYQKKPGKAPELLIYYSTLLVDGVPDRFSGSRSGTDFTLTISSLQPEDFATYYCQHYYSIPYTFGQGTKVEIERTVAAPSVFIFPPSDKQLKSGTAKVVCLLNNFYPREAKVQWKVDNALQSGNSKESVTEQDSKDSTYSLSSTLTLSKADYEKHKVYACEVTHQGLSSPVTKSFNRGEC

SEQ ID NO: 51 - protein, 214 aa, Artificial sequence
  an artificially synthesized sequence
DIQMTQSPSSLSASVGDRVTITCRASYNIDSYLAWYQQKPGKAPELLIYYSTLLVDGVPDRFSGSRSGTDFTLTISSLQPEDFATYYCQHYYSIPYTFGQGTKVEIERTVAAPSVFIFPPSDEQLKSGTAKVVCLLNNFYPREAKVQWKVDNALQSGNSKESVTEQDSKDSTYSLSSTLTLSKADYEKHKVYACEVTHQGLSSPVTKSFNRGEC

SEQ ID NO: 52 - protein, 226 aa, Artificial sequence
  an artificially synthesized sequence
QVQLVESGGGVVQPGGSLRLSCAASGFTFSSYTMSWVRQAPGQGLEWMGTISSGGGRTYYPDSVKGRFTISRDNSKNTLYLQMNSLRDEDTAVYYCARGDYRYDGFAYWGEGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 214 aa, Artificial sequence
  an artificially synthesized sequence
DIQMTQSPSSLSASVGDRVTITCRASYNIDSYLAWYQQKPGKAPELLIYYSTLLVDGVPDRFSGSRSGTDFTLTISSLQPEDFATYYCQHYYSIPYTFGQGTKVEIERTVAAPSVFIFPPSDKQLKSGTAKVVCLLNNFYPREAKVQWKVDNALQSGNSKESVTEQDSKDSTYSLSSTLTLSKADYEKHKVYACEVTHQGLSSPVTKSFNRGEC

SEQ ID NO: 54 - protein, 456 aa, Artificial sequence
  an artificially synthesized sequence
QVQLVESGGGLVQPGRSLRLSCAASGFKFSNVWFHWVRKAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 55 - protein, 456 aa, Artificial sequence
  an artificially synthesized sequence
QVQLVESGGGLVQPGRSLRLSCAASGFKFSNVWFHWVRKAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 56 - protein, 456 aa, Artificial sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 57 - protein, 456 aa, Artificial sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 58 - protein, 456 aa, Artificial sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 59 - protein, 456 aa, Artificial sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 60 - protein, 456 aa, Artificial sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 61 - protein, 456 aa, Artificial sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 62 - protein, 456 aa, Artificial sequence
  an artificially synthesized sequence
QVQLVESGGGLVQPGRSLRLSCAASGFKFSNVWFHWVRKAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 63 - protein, 456 aa, Artificial sequence
  an artificially synthesized sequence
QVQLVESGGGLVQPGRSLRLSCAASGFKFSNVWFHWVRKAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 64 - protein, 456 aa, Artificial sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 65 - protein, 456 aa, Artificial sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 66 - protein, 456 aa, Artificial sequence
  an artificially synthesized sequence
QVQLVESGGGLVQPGRSLRLSCAASGFKFSNVWFHWVRQAPGKGLEWVAQIKDYYNAYAAYYAPSVKGRFTISRDDSKNSIYLQMNSLKTEDTAVYYCHYVHYASASTLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 67 - protein, 456 aa, Artificial sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASQLLPAEGVDAWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 68 - protein, 219 aa, Artificial sequence
  an artificially synthesized sequence
DIVMTQSPLSLPVTPGEPASISCQPSQEVVHMNRNTYLHWYQEKPGQAPRLLIYKVSNRFPGVPDRFSGSGSGTDFTLKISRVEAEDVGVYYCAQGTSHPFTFGQGTKLEIKRTVAAPSVFIFPPSDEQLKSGTAEVVCLLNNFYPREAKVQWKVDNALQSGNSEESVTEQDSKDSTYSLSSTLELSKADYEKHKVYACEVTHQGLSSPVTKSFNRGEC

SEQ ID NO: 69 - protein, 219 aa, Artificial sequence
  an artificially synthesized sequence
DIVMTQSPLSLPVTPGEPASISCQPSQEVVHMNRNTYLHWYQQKPGQAPRLLIYKVSNRFPGVPDRFSGSGSGTDFTLKISRVEAEDVGVYYCAQGTSHPFTFGQGTKLEIKRTVAAPSVFIFPPSDEQLKSGTAEVVCLLNNFYPREAKVQWKVDNALQSGNSEESVTEQDSKDSTYSLSSTLTLSKADYEKHKVYACEVTHQGLSSPVTKSFNRGEC

SEQ ID NO: 70 - protein, 219 aa, Artificial sequence
  an artificially synthesized sequence
DIVMTQSPLSLPVTPGEPASISCQPSQEVVHMNRNTYLHWYQQKPGQAPRLLIYKVSNRFPGVPDRFSGSGSGTDFTLKISRVEAEDVGVYYCAQGTSHPFTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 219 aa, Artificial sequence
  an artificially synthesized sequence
DIVMTQSPLSLPVTPGEPASISCQPSQEVVHMNRNTYLHWYQQKPGQAPRLLIYKVSNRFPGVPDRFSGSGSGTDFTLKISRVEAEDVGVYYCAQGTSHPFTFGQGTKLEIKRTVAAPSVFIFPPSDEQLKSGTAEVVCLLNNFYPREAKVQWKVDNALQSGNSEESVTEQDSKDSTYSLSSTLELSKADYEKHKVYACEVTHQGLSSPVTKSFNRGEC

SEQ ID NO: 72 - protein, 107 aa, Artificial sequence
  an artificially synthesized sequence
RTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 328 aa, Artificial sequence
  an artificially synthesized sequence
ASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 74 - protein, 328 aa, Artificial sequence
  an artificially synthesized sequence
ASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 75 - protein, 328 aa, Artificial sequence
  an artificially synthesized sequence
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 76 - protein, 330 aa, Artificial sequence
  an artificially synthesized sequence
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQEGNVFSCSVMHEALHNHYTQKSLSLSP

SEQ ID NO: 77 - protein, 328 aa, Artificial sequence
  an artificially synthesized sequence
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHAHYTRKELSLSP

SEQ ID NO: 78 - protein, 330 aa, Artificial sequence
  an artificially synthesized sequence
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHAHYTRKELSLSP

SEQ ID NO: 79 - protein, 328 aa, Artificial sequence
  an artificially synthesized sequence
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 80 - protein, 330 aa, Artificial sequence
  an artificially synthesized sequence
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 81 - protein, 328 aa, Artificial sequence
  an artificially synthesized sequence
ASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 82 - protein, 328 aa, Artificial sequence
  an artificially synthesized sequence
ASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDERVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTREELSLSP

SEQ ID NO: 83 - protein, 328 aa, Artificial sequence
  an artificially synthesized sequence
ASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 84 - protein, 328 aa, Artificial sequence
  an artificially synthesized sequence
ASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTREELSLSP

SEQ ID NO: 85 - protein, 107 aa, Artificial sequence
  an artificially synthesized sequence
RTVAAPSVFIFPPSDEQLKSGTAEVVCLLNNFYPREAKVQWKVDNALQSGNSEESVTEQDSKDSTYSLSSTLTLSKADYEKHKVYACEVTHQGLSSPVTKSFNRGEC

SEQ ID NO: 86 - protein, 107 aa, Artificial sequence
  an artificially synthesized sequence
RTVAAPSVFIFPPSDEQLKSGTAKVVCLLNNFYPREAKVQWKVDNALQSGNSKESVTEQDSKDSTYSLSSTLTLSKADYEKHKVYACEVTHQGLSSPVTKSFNRGEC

SEQ ID NO: 87 - protein, 107 aa, Artificial sequence
  an artificially synthesized sequence
RTVAAPSVFIFPPSDEQLKSGTAEVVCLLNNFYPREAKVQWKVDNALQSGNSEESVTEQDSKDSTYSLSSTLELSKADYEKHKVYACEVTHQGLSSPVTKSFNRGEC

SEQ ID NO: 88 - protein, 107 aa, Artificial sequence
  an artificially synthesized sequence
RTVAAPSVFIFPPSDKQLKSGTAKVVCLLNNFYPREAKVQWKVDNALQSGNSKESVTEQDSKDSTYSLSSTLTLSKADYEKHKVYACEVTHQGLSSPVTKSFNRGEC

SEQ ID NO: 89 - protein, 107 aa, Artificial sequence
  an artificially synthesized sequence
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 128 aa, Artificial sequence
  an artificially synthesized sequence
QVQLVESGGGLVQPGRSLRLSCAASGFTFSNAWMHWVRQAPGKGLEWVAQIKDKGNAYAAYYAPSVKGRFTISRDDSKNSIYLQMNSLKTEDTAVYYCHYVHYASASTVLPAFGVDAWGQGTTVTVSS

SEQ ID NO: 91 - protein, 128 aa, Artificial sequence
  an artificially synthesized sequence
QVQLVESGGGLVQPGRSLRLSCAASGFKFSNVWMHWVRQAPGKGLEWVAQIKDKYNAYAAYYAPSVKGRFTISRDDSKNSIYLQMNSLKTEDTAVYYCHYVHYASASTLLPAFGVDAWGQGTTVTVSS

SEQ ID NO: 92 - protein, 128 aa, Artificial sequence
  an artificially synthesized sequence
QVQLVESGGGLVQPGRSLRLSCAASGFKFSNVWMHWVRQAPGKGLEWVAQIKDKYNAYAAYYAPSVKGRFTISRDDSKNSIYLQMNSLKTEDTAVYYCHYIHYASASTLLPAFGVDAWGQGTTVTVSS

SEQ ID NO: 93 - protein, 128 aa, Artificial sequence
  an artificially synthesized sequence
QVQLVESGGGLVQPGRSLRLSCAASGFKFSNVWFHWVRQAPGKGLEWVAQIKDKYNAYATYYAPSVKGRFTISRDDSKNSIYLQMNSLKTEDTAVYYCHYVHYASASTLLPAFGVDAWGQGTTVTVSS

SEQ ID NO: 94 - protein, 128 aa, Artificial sequence
  an artificially synthesized sequence
QVQLVESGGGLVQPGRSLRLSCAASGFVFSNVWMHWVRQAPGKGLEWVAQIKDKWNAYAAYYAPSVKGRFTISRDDSKNSIYLQMNSLKTEDTAVYYCHYIHYASASTLLPAEGIDAWGQGTTVTVSS

SEQ ID NO: 95 - protein, 128 aa, Artificial sequence; identical to SEQ ID NO: 5
  an artificially synthesized sequence

SEQ ID NO: 96 - protein, 128 aa, Artificial sequence
  an artificially synthesized sequence
QVQLVESGGGLVQPGRSLRLSCAASGFKFSNTWFHWVRQAPGKGLEWVAQIKDYYNDYAAYYAPSVKGRFTISRDDSKNSIYLQMNSLKTEDTAVYYCHYVHYASASTLLPAEGVDAWGQGTTVTVSS

SEQ ID NO: 97 - protein, 128 aa, Artificial sequence
  an artificially synthesized sequence
QVQLVESGGGLVQPGRSLRLSCAASGFVFSNVWFHWVRQAPGKGLEWVAQIKDKYNAYADYYAPSVKERFTISRDDSKNSIYLQMNSLKTEDTAVYYCHYVHYASASTLLPAEGVDAWGQGTTVTVSS

SEQ ID NO: 98 - protein, 128 aa, Artificial sequence
  an artificially synthesized sequence
QVQLVESGGGLVQPGRSLRLSCAASGFVFSNVWFHWVRQAPGKGLEWVAQIKDKYNAYADYYAPSVEGRFTISRDDSKNSIYLQMNSLKTEDTAVYYCHYVHYASASTLLPAEGVDAWGQGTTVTVSS

SEQ ID NO: 99 - protein, 128 aa, Artificial sequence
  an artificially synthesized sequence
QVQLVESGGGLVQPGRSLRLSCAASGFVFSNVWFHWVRQAPGKGLEWVAQIKDYYNAYADYYAPSVEGRFTISRDDSKNSIYLQMNSLKTEDTAVYYCHYVHYASASTLLPAEGVDAWGQGTTVTVSS

SEQ ID NO: 100 - protein, 128 aa, Artificial sequence
  an artificially synthesized sequence
QVQLVESGGGLVQPGRSLRLSCAASGFVFSNVWFHWVRQAPGKGLEWVAQIKDKWNAYADYYAPSVKERFTISRDDSKNSIYLQMNSLKTEDTAVYYCHYIHYASASTLLPAEGIDAWGQGTTVTVSS

SEQ ID NO: 101 - protein, 128 aa, Artificial sequence
  an artificially synthesized sequence
QVQLVESGGGLVQPGRSLRLSCAASGFKFSNVWFHWVRQAPGKGLEWVAQIKDYYNAYAGYYHPSVKGRFTISRDDSKNSIYLQMNSLKTEDTAVYYCHYVHYAAASTLLPAEGVDAWGQGTTVTVSS

SEQ ID NO: 102 - protein, 128 aa, Artificial sequence; identical to SEQ ID NO: 6
  an artificially synthesized sequence

SEQ ID NO: 103 - protein, 5 aa, Artificial sequence
  an artificially synthesized sequence
NAWMH

SEQ ID NO: 104 - protein, 5 aa, Artificial sequence
  an artificially synthesized sequence
NVWMH

SEQ ID NO: 105 - protein, 5 aa, Artificial sequence
  an artificially synthesized sequence
NVWMH

SEQ ID NO: 106 - protein, 5 aa, Artificial sequence
  an artificially synthesized sequence
NVWFH

SEQ ID NO: 107 - protein, 5 aa, Artificial sequence
  an artificially synthesized sequence
NVWMH

SEQ ID NO: 108 - protein, 5 aa, Artificial sequence
  an artificially synthesized sequence
NVWFH

SEQ ID NO: 109 - protein, 5 aa, Artificial sequence
  an artificially synthesized sequence
NTWFH

SEQ ID NO: 110 - protein, 5 aa, Artificial sequence
  an artificially synthesized sequence
NVWFH

SEQ ID NO: 111 - protein, 5 aa, Artificial sequence
  an artificially synthesized sequence
NVWFH

SEQ ID NO: 112 - protein, 5 aa, Artificial sequence
  an artificially synthesized sequence
NVWFH

SEQ ID NO: 113 - protein, 5 aa, Artificial sequence
  an artificially synthesized sequence
NVWFH

SEQ ID NO: 114 - protein, 5 aa, Artificial sequence
  an artificially synthesized sequence
NVWFH

SEQ ID NO: 115 - protein, 5 aa, Artificial sequence
  an artificially synthesized sequence
NVWFH

SEQ ID NO: 116 - protein, 19 aa, Artificial sequence
  an artificially synthesized sequence
QIKDKGNAYAAYYAPSVKG

SEQ ID NO: 117 - protein, 19 aa, Artificial sequence
  an artificially synthesized sequence
QIKDKYNAYAAYYAPSVKG

SEQ ID NO: 118 - protein, 19 aa, Artificial sequence
  an artificially synthesized sequence
QIKDKYNAYAAYYAPSVKG

SEQ ID NO: 119 - protein, 19 aa, Artificial sequence
  an artificially synthesized sequence
QIKDKYNAYATYYAPSVKG

SEQ ID NO: 120 - protein, 19 aa, Artificial sequence
  an artificially synthesized sequence
QIKDKWNAYAAYYAPSVKG

SEQ ID NO: 121 - protein, 19 aa, Artificial sequence
  an artificially synthesized sequence
QIKDYYNAYAAYYAPSVKG

SEQ ID NO: 122 - protein, 19 aa, Artificial sequence
  an artificially synthesized sequence
QIKDYYNDYAAYYAPSVKG

SEQ ID NO: 123 - protein, 19 aa, Artificial sequence
  an artificially synthesized sequence
QIKDKYNAYADYYAPSVKE

SEQ ID NO: 124 - protein, 19 aa, Artificial sequence
  an artificially synthesized sequence
QIKDKYNAYADYYAPSVEG

SEQ ID NO: 125 - protein, 19 aa, Artificial sequence
  an artificially synthesized sequence
QIKDYYNAYADYYAPSVEG

SEQ ID NO: 126 - protein, 19 aa, Artificial sequence
  an artificially synthesized sequence
QIKDKWNAYADYYAPSVKE

SEQ ID NO: 127 - protein, 19 aa, Artificial sequence
  an artificially synthesized sequence
QIKDYYNAYAGYYHPSVKG

SEQ ID NO: 128 - protein, 19 aa, Artificial sequence
  an artificially synthesized sequence
QIKDYYNAYAGYYHPSVKG

SEQ ID NO: 129 - protein, 17 aa, Artificial sequence
  an artificially synthesized sequence
VHYASASTVLPAFGVDA

SEQ ID NO: 130 - protein, 17 aa, Artificial sequence
  an artificially synthesized sequence
VHYASASTLLPAFGVDA

SEQ ID NO: 131 - protein, 17 aa, Artificial sequence
  an artificially synthesized sequence
IHYASASTLLPAFGVDA

SEQ ID NO: 132 - protein, 17 aa, Artificial sequence
  an artificially synthesized sequence
VHYASASTLLPAFGVDA

SEQ ID NO: 133 - protein, 17 aa, Artificial sequence
  an artificially synthesized sequence
IHYASASTLLPAEGIDA

SEQ ID NO: 134 - protein, 17 aa, Artificial sequence
  an artificially synthesized sequence
VHYASASTLLPAEGVDA

SEQ ID NO: 135 - protein, 17 aa, Artificial sequence
  an artificially synthesized sequence
VHYASASTLLPAEGVDA

SEQ ID NO: 136 - protein, 17 aa, Artificial sequence
  an artificially synthesized sequence
VHYASASTLLPAEGVDA

SEQ ID NO: 137 - protein, 17 aa, Artificial sequence
  an artificially synthesized sequence
VHYASASTLLPAEGVDA

SEQ ID NO: 138 - protein, 17 aa, Artificial sequence
  an artificially synthesized sequence
VHYASASTLLPAEGVDA

SEQ ID NO: 139 - protein, 17 aa, Artificial sequence
  an artificially synthesized sequence
IHYASASTLLPAEGIDA

SEQ ID NO: 140 - protein, 17 aa, Artificial sequence
  an artificially synthesized sequence
VHYAAASTLLPAEGVDA

SEQ ID NO: 141 - protein, 17 aa, Artificial sequence
  an artificially synthesized sequence
VHYAAASQLLPAEGVDA

SEQ ID NO: 142 - protein, 112 aa, Artificial sequence
  an artificially synthesized sequence
DIVMTQSPLSLPVTPGEPASISCQASQELVHMNRNTYLHWYQQKPGQAPRLLIYKVSNRFPGVPDRFSGSGSGTDFTLKISRVEAEDVGVYYCAQGTSVPFTFGQGTKLEIK

SEQ ID NO: 143 - protein, 112 aa, Artificial sequence; identical to SEQ ID NO: 28
  an artificially synthesized sequence

SEQ ID NO: 144 - protein, 112 aa, Artificial sequence
  an artificially synthesized sequence
DIVMTQSPLSLPVTPGEPASISCQPSQEVVHMNNVVYLHWYQQKPGQAPRLLIYKVSNRFPGVPDRFSGSGSGTDFTLKISRVEAEDVGVYYCAQGTSHPFTFGQGTKLEIK

SEQ ID NO: 145 - protein, 112 aa, Artificial sequence
  an artificially synthesized sequence
DIVMTQSPLSLPVTPGEPASISCQPSQEVVHMNRNTYLHWYQQKPGQAPRLLIYKVSNVFPGVPDRFSGSGSGTDFTLKISRVEAEDVGVYYCAQGTHHPFTFGQGTKLEIK

SEQ ID NO: 146 - protein, 112 aa, Artificial sequence
  an artificially synthesized sequence
DIVMTQSPLSLPVTPGEPASISCQPSEEVVHMNRNTYLHWYQQKPGQAPRLLIYKVSNLFPGVPDRFSGSGSGTDFTLKISRVEAEDVGVYYCAQGTHHPFTFGQGTKLEIK

SEQ ID NO: 147 - protein, 16 aa, Artificial sequence
  an artificially synthesized sequence
QASQELVHMNRNTYLH

SEQ ID NO: 148 - protein, 16 aa, Artificial sequence
  an artificially synthesized sequence
QPSQEVVHMNRNTYLH

SEQ ID NO: 149 - protein, 16 aa, Artificial sequence
  an artificially synthesized sequence
QPSQEVVHMNNVVYLH

SEQ ID NO: 150 - protein, 16 aa, Artificial sequence
  an artificially synthesized sequence
QPSQEVVHMNRNTYLH

SEQ ID NO: 151 - protein, 16 aa, Artificial sequence
  an artificially synthesized sequence
QPSEEVVHMNRNTYLH

SEQ ID NO: 152 - protein, 7 aa, Artificial sequence
  an artificially synthesized sequence
KVSNRFP

SEQ ID NO: 153 - protein, 7 aa, Artificial sequence
  an artificially synthesized sequence
KVSNRFP

SEQ ID NO: 154 - protein, 7 aa, Artificial sequence
  an artificially synthesized sequence
KVSNRFP

SEQ ID NO: 155 - protein, 7 aa, Artificial sequence
  an artificially synthesized sequence
KVSNVFP

SEQ ID NO: 156 - protein, 7 aa, Artificial sequence
  an artificially synthesized sequence
KVSNLFP

SEQ ID NO: 157 - protein, 9 aa, Artificial sequence
  an artificially synthesized sequence
AQGTSVPFT

SEQ ID NO: 158 - protein, 9 aa, Artificial sequence
  an artificially synthesized sequence
AQGTSHPFT

SEQ ID NO: 159 - protein, 9 aa, Artificial sequence
  an artificially synthesized sequence
AQGTSHPFT

SEQ ID NO: 160 - protein, 9 aa, Artificial sequence
  an artificially synthesized sequence
AQGTHHPFT

SEQ ID NO: 161 - protein, 9 aa, Artificial sequence
  an artificially synthesized sequence
AQGTHHPFT

SEQ ID NO: 162 - protein, 122 aa, Artificial sequence
  an artificially synthesized sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSS

SEQ ID NO: 163 - protein, 112 aa, Artificial sequence
  an artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIK

SEQ ID NO: 164 - protein, 121 aa, Artificial sequence [sequence omitted]

SEQ ID NO: 165 - protein, 109 aa, Artificial sequence [sequence omitted]

SEQ ID NO: 166 - protein, 399 aa, Artificial sequence
  an artificially synthesized sequence
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 167 - protein, 444 aa, Artificial sequence
  an artificially synthesized sequence
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSFEDPEVKFNWYVDGVEVHNAKTKPREEPYNSTYRVVSVLTVLHQDWLNGKEYKCKVSNKAAPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 168 - protein, 214 aa, Artificial sequence
  an artificially synthesized sequence
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 169 - protein, 212 aa, Artificial sequence
  an artificially synthesized sequence
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVGSADDAKKDAAKKDDAKKDDAKKDGSQSIKGNHLVKVYDYQEDGSVLLTCDAEAKNITWFKDGKMIGFLTEDKKKWNLGSNAKDPRGMYQCKGSQNKSKPLQVYYRMDYKDDDDK

SEQ ID NO: 170 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 171 - protein, 4 aa, Artificial sequence
  an artificially synthesized sequence
GGGS

SEQ ID NO: 172 - protein, 4 aa, Artificial sequence
  an artificially synthesized sequence
SGGG

SEQ ID NO: 173 - protein, 5 aa, Artificial sequence
  an artificially synthesized sequence
GGGGS

SEQ ID NO: 174 - protein, 5 aa, Artificial sequence
  an artificially synthesized sequence
SGGGG

SEQ ID NO: 175 - protein, 6 aa, Artificial sequence
  an artificially synthesized sequence
GGGGGS

SEQ ID NO: 176 - protein, 6 aa, Artificial sequence
  an artificially synthesized sequence
SGGGGG

SEQ ID NO: 177 - protein, 7 aa, Artificial sequence
  an artificially synthesized sequence
GGGGGGS

SEQ ID NO: 178 - protein, 7 aa, Artificial sequence
  an artificially synthesized sequence
SGGGGGG

SEQ ID NO: 179 - protein, 189 aa, Artificial sequence
  an artificially synthesized sequence
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQHHHHHHGGGGSGLNDIFEAQKIEWHE

SEQ ID NO: 180 - protein, 22 aa, Artificial sequence
  an artificially synthesized sequence
LQDPCSNCPAGTFCDNNRNQIC

SEQ ID NO: 181 - protein, 31 aa, Artificial sequence
  an artificially synthesized sequence
DCTPGFHCLGAGCSMCEQDCKQGQELTKKGC

SEQ ID NO: 182 - protein, 72 aa, Artificial sequence
  an artificially synthesized sequence
SPCPPNSFSSAGGQRTCDICRQCKGVFRTRKECSSTSNAECDCTPGFHCLGAGCSMCEQDCKQGQELTKKGC

SEQ ID NO: 183 - protein, 63 aa, Artificial sequence
  an artificially synthesized sequence
LQDPCSNCPAGTFCDNNRNQICSPCPPNSFSSAGGQRTCDICRQCKGVFRTRKECSSTSNAEC

SEQ ID NO: 184 - protein, 122 aa, Artificial sequence
  an artificially synthesized sequence
QVQLVESGGGVVQPGGSLRLSCAASGFTFSNAWMHWVRKAPGKGLEWVAQIKDKSQNYATYVAESVKGRFTISRADSKNSIYLQMNSLKTEDTAVYYCRYVHYAAGYGVDIWGQGTTVTVSS

SEQ ID NO: 185 - protein, 112 aa, Artificial sequence
  an artificially synthesized sequence
DIVMTQSPLSLPVTPGEPASISCRSSQPLVHSNRNTYLHWYQEKPGQAPRLLIYKVSNRFSGVPDRFSGSGSGTDFTLKISRVEAEDVGVYYCGQGTQVPYTFGQGTKLEIK

SEQ ID NO: 186 - protein, 116 aa, Artificial sequence
  an artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSS

SEQ ID NO: 187 - protein, 107 aa, Artificial sequence
  an artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVK

SEQ ID NO: 188 - protein, 122 aa, Artificial sequence; identical to SEQ ID NO: 162
  an artificially synthesized sequence

SEQ ID NO: 189 - protein, 112 aa, Artificial sequence; identical to SEQ ID NO: 163
  an artificially synthesized sequence

SEQ ID NO: 190 - protein, 117 aa, Artificial sequence
  an artificially synthesized sequence
EVQLQQSGPELVKPGASMKISCKASGYSFTGYTMNWVKQSHGKNLEWIGLINPYNGGTIYNQKFKGKATLTVDKSSSTAYMELLSLTSEDSAVYYCARDYGFVLDYWGQGTTLTVSS

SEQ ID NO: 191 - protein, 107 aa, Artificial sequence
  an artificially synthesized sequence
DIVLTQSPSIMSVSPGEKVTITCSASSSVSYMHWFQQKPGTSPKLCIYSTSNLASGVPARFSGRGSGTSYSLTISRVAAEDAATYYCQQRSNYPPWTFGGGTKLEIK

SEQ ID NO: 192 - protein, 219 aa, Artificial sequence
  an artificially synthesized sequence
DIVMTQSPLSLPVTPGEPASISCRSSQPLVHSNRNTYLHWYQEKPGQAPRLLIYKVSNRFSGVPDRFSGSGSGTDFTLKISRVEAEDVGVYYCGQGTQVPYTFGQGTKLEIKRTVAAPSVFIFPPSDEQLKSGTAEVVCLLNNFYPREAKVQWKVDNALQSGNSEESVTEQDSKDSTYSLSSTLELSKADYEKHKVYACEVTHQGLSSPVTKSFNRGEC

SEQ ID NO: 193 - protein, 447 aa, Artificial sequence; identical to SEQ ID NO: 41
  an artificially synthesized sequence

SEQ ID NO: 194 - protein, 450 aa, Artificial sequence
  an artificially synthesized sequence
QVQLVESGGGVVQPGGSLRLSCAASGFTFSNAWMHWVRKAPGKGLEWVAQIKDKSQNYATYVAESVKGRFTISRADSKNSIYLQMNSLKTEDTAVYYCRYVHYAAGYGVDIWGQGTTVTVSSASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 195 - protein, 214 aa, Artificial sequence; identical to SEQ ID NO: 50
  an artificially synthesized sequence

SEQ ID NO: 196 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAVIRDFYNPLVAEAQKRELGASLYLGWAASGLLLLGGGLLCCTCPSGGSQGPSHYMARYSTSAPAISRGPSEYPTKNYV

SEQ ID NO: 197 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 198 - protein, 217 aa, Homo sapiens
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 199 - protein, 220 aa, Homo sapiens
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 200 - protein, 209 aa, Homo sapiens
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 201 - protein, 219 aa, Mus musculus
MASTGLQILGIVLTLLGWVNALVSCALPMWKVTAFIGNSIVVAQMVWEGLWMSCVVQSTGQMQCKVYDSLLALPQDLQAARALCVVTLLIVLLGLLVYLAGAKCTTCVEDRNSKSRLVLISGIIFVISGVLTLIPVCWTAHSIIQDFYNPLVADAQKRELGASLYLGWAASGLLLLGGGLLCCACSSGGTQGPRHYMACYSTSVPHSRGPPEYPTKNYV

SEQ ID NO: 202 - protein, 217 aa, Mus musculus
MASTGLELLGMTLAVLGWLGTLVSCALPLWKVTAFIGNSIVVAQVVWEGLWMSCVVQSTGQMQCKVYDSLLALPQDLQAARALCVVALLLALLGLLVAITGAQCTTCVEDEGAKARIVLTAGVLLLLSGILVLIPVCWTAHAIIQDFYNPLVAEALKRELGASLYLGWAAAALLMLGGGLLCCTCPPSHFERPRGPRLGYSIPSRSGASGLDKRDYV

SEQ ID NO: 203 - protein, 219 aa, Mus musculus
MSMGLEITGTSLAVLGWLCTIVCCALPMWRVSAFIGSSIITAQITWEGLWMNCVVQSTGQMQCKMYDSLLALPQDLQAARALIVVSILLAAFGLLVALVGAQCTNCVQDETAKAKITIVAGVLFLLAALLTLVPVSWSANTIIRDFYNPLVPEAQKREMGAGLYVGWAAAALQLLGGALLCCSCPPRDKYAPTKILYSAPRSTGPGTGTGTAYDRKDYV

SEQ ID NO: 204 - protein, 210 aa, Mus musculus
MASMGLQVLGISLAVLGWLGIILSCALPMWRVTAFIGSNIVTAQTSWEGLWMNCVVQSTGQMQCKMYDSMLALPQDLQAARALMVISIIVGALGMLLSVVGGKCTNCMEDETVKAKIMITAGAVFIVASMLIMVPVSWTAHNVIRDFYNPMVASGQKREMGASLYVGWAASGLLLLGGGLLCCSCPPRSNDKPYSAKYSAARSVPASNYV

SEQ ID NO: 205 - protein, 220 aa, Artificial sequence
  an artificially synthesized sequence
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEALKRELGASLYLGWAASGLLLLGGGLLCCTCPSGGSQGPSHYMARYSTSAPAISRGPSEYPTKNYV

(19) United States
(12) Patent Application Publication
(10) Pub. No.: US 2023/0220066 A1
(43) Pub. Date: Jul. 13, 2023

(54) CLAUDIN-6 TARGETING MULTISPECIFIC ANTIGEN-BINDING MOLECULES AND USES THEREOF

(71) Applicant: Chugai Seiyaku Kabushiki Kaisha, Tokyo (JP)

(72) Inventors: Shinya ISHII, Kanagawa (JP); Naoki KIMURA, Kanagawa (JP); Tatsushi KODAMA, Tokyo (JP)

(21) Appl. No.: 17/913,899

(22) PCT Filed: Mar. 30, 2021
(86) PCT No.: PCT/JP2021/013526

§ 371 Date: Sep. 23, 2022

(30) Foreign Application Priority Data
Mar. 31, 2020 (JP) 2020-062881
Apr. 16, 2020 (JP) 2020-073335

(51) Int. Cl. C07K 16/28; A61P 35/00

(52) U.S. Cl. CPC C07K 16/28; C07K 16/2803; C07K 16/2878; A61P 35/00; C07K 2317/31; A61K 39/00

(57) ABSTRACT

The present disclosure provides multispecific antigen-binding molecules capable of binding to CD3 and CD137 (4-1BB) but not binding to CD3 and CD137 at the same time, and capable of binding to CLDN6. The multispecific antigen-binding molecules of the present disclosure exhibit enhanced T-cell dependent cytotoxicity activity in a CLDN6-dependent manner through binding to the CD3/CD37 and CLDN6. The present invention provides multi-specific antigen-binding molecules and pharmaceutical compositions thereof that can be used for targeting cells expressing CLDN6, for use in immunotherapy for treating various cancers, especially those associated with CLDN6 such as CLDN6-positive cancers.

### CROSS-REFERENCE TO RELATED APPLICATIONS

This application is a U.S. National Phase of PCT Application No. PCT/JP2021/013526, filed Mar. 30, 2021, which claims the benefit of Japanese Patent Application No. 2020-062881, filed Mar. 31, 2020, and Japanese Patent Application No. 2020-073335, filed Apr. 16, 2020, each of which is incorporated herein by reference in its entirety.

### REFERENCE TO SEQUENCE LISTING SUBMITTED ELECTRONICALLY

The content of the electronically submitted sequence listing (Name: 6663_0213 Sequence_Listing.txt; Size: 258 KB; and Date of Creation: Sep. 22, 2022) filed with the application is incorporated herein by reference in its entirety.

### TECHNICAL FIELD

The present disclosure relates to Claudin-6 targeting multispecific antigen-binding molecules, uses thereof, and such.

### BACKGROUND ART

Claudin family is the family of cell membrane proteins of approximately 23 kD in molecular weight which have four transmembrane domains and constitute tight junctions. The Claudin family includes 24 members in humans and mice, and each member of the Claudin family is known to exhibit a very unique expression pattern depending on each epithelial cell type (NPL 1 to NPL 4). In the sheet of epithelial cells, a mechanism works to prevent substances from leaking (diffusing) in the intercellular spaces, and cell-cell adhesion systems called tight junctions have been shown to really play a central role as a “barrier” in the mechanism to prevent leakage.

A tight junction molecule Claudin 6 (CLDN6), a member of Claudin family proteins, shows transcriptionally silent expression in normal adult tissues (NPL 5 and NPL 6), while showing up-regulation in several kind of cancers such as ovarian cancer, NSCLC, and gastric cancers (NPL 7 to NPL 9).

Regarding anti-CLDN6 antibodies, monospecific antibodies against CLDN6 have been reported to have ADCC activity or internalization activity against CLDN6 positive cancer lines (PTL 1 to PTL 5). So far, CLDN6 targeting T cell-redirecting bispecific antibodies, named as 6PHU3, has been engineered using bispecific sc(Fv)₂ format with anti-CD3/anti-CLDN6 specificities (PTL 6 to PTL7). In preclinical evaluation, 6PHU3 has been reported to show a potent killing of cancer cells in vitro and in vivo (NPL 10).

### CITATION LIST

### Patent Literature

[PTL 1] WO2009/087978
[PTL 2] WO2011/057788
[PTL 3] WO2012/003956
[PTL 4] WO2012/156018
[PTL 5] WO2015/069794
[PTL 6] WO2014/075697
[PTL 7] WO2014/075788

### Non Patent Literature

[NPL 1] Furuse and Tsukita, TRENDS in Cell Biology 2006, 16: 181
[NPL 2] Wilcox, et al., Cell 2001, 104: 165
[NPL 3] Rahner, et al., GASTROENTEROLOGY 2001, 120: 411
[NPL 4] Morita, et al., Proc. Natl. Acad. Sci. USA 1999, 96: 511
[NPL 5] Dev Dyn. 2004 October; 231(2):425-31.
[NPL 6] Am J Physiol Renal Physiol. 2006 December; 291(6): F1132-41.
[NPL 7] Int J Cancer. 2014 Nov. 1; 135(9):2206-14.
[NPL 8] Histopathology. 2012 December; 61(6):1043-56.
[NPL 9] J Gastrointest Cancer. 2010 March; 41(1):52-9.
[NPL 10] Oncoimmunology. 2015 Oct. 29; 5(3):e1091555.

### SUMMARY OF INVENTION

### Technical Problem

An objective of the present disclosure is to provide multispecific antigen-binding molecules that can recruit T cells efficiently and specifically to the target cancer cells, especially CLDN6-expressing cells such as cancer cells, and can treat cancer through the cytotoxic activity of T cells against target cancer tissues containing CLDN6-expressing cells; methods for producing the antigen-binding molecules; and pharmaceutical compositions comprising the antigen-binding molecules as active ingredient. The invention also provides methods to obtain multispecific antigen-binding molecules which induce T-cell dependent cytotoxicity more efficiently whilst circumventing adverse toxicity concerns or side effects that prior art multispecific antigen-binding molecules may have.

### Solutions to Problem

Specifically, the present disclosure provides an antigen-binding molecule comprising: a first antigen-binding moiety that is capable of binding to CD3 and CD137 (4-1BB), but does not bind to CD3 and CD137 at the same time (i.e. dual-binding to CD3 and CD137 but not simultaneously); and a second antigen-binding moiety capable of binding to a molecule specifically expressed in a cancer tissue, specifically Claudin-6 (CLDN6).

Advantageously, by having a Dual binding capability to CD137 in addition to binding capability to CD3, the multispecific antigen-binding molecule of the present disclosure exhibits enhanced T-cell dependent cytotoxicity activity contributed by the synergistic co-stimulator CD137 signaling on the CD3 signaling, compared to a T-cell recruiting bispecific antibody which binds to CD3 alone. In addition, since the binding of the antigen-binding molecule to CD3 and CD137 is non-simultaneous (i.e. not binding to CD3 and CD137 at the same time), the simultaneous binding of CD3 and/or CD137 expressed on different immune cells (e.g. T cells) by the same antigen-binding molecule will not occur, thereby circumventing systemic toxicity concerns due to undesirable cross-linking between different immune cells which is considered to be responsible for adverse reactions when a conventional multispecific antigen-binding molecule capable of simultaneously binding to CD3 and a second molecule expressed on T cells (e.g. CD137) is administered in vivo.

Additionally, by engineering and improving the binding activity to CD137 without adversely affecting the Dual-binding activity to CD3 of the antigen-binding molecules of the present disclosure, the inventors have selected, out of more than 1000 variants, antigen binding molecules comprising specific heavy chain complementarity determining regions (HCDRs) or heavy chain variable region (VH) together with specific light chain complementarity determining regions (LCDRs) or light chain variable region (VL), that exhibit superior T-cell dependent cytotoxicity activity to tumors in a cancer antigen (CLDN6)-dependent manner. In one aspect, the inventors surprisingly found that, by engineering the optimal CD3 and CD137 binding profile, the selected antigen-binding molecules exhibit strong T-cell dependent cytotoxicity activity with low toxicity.

Finally, a common challenge in the development of multispecific antibodies has been the production of multispecific antibody constructs at a clinically sufficient quantity and purity, due to the mispairing of antibody heavy and light chains of different specificities upon co-expression, which decreases the yield of the correctly assembled construct and results in a number of non-functional side products from which the desired multispecific antibody may be difficult to separate. In one aspect, by way of careful antibody engineering and molecular format design (including charged mutations in the constant region, VH/VL exchanged, and Fc region selection), the present disclosure provides multispecific antigen-binding molecules designed for T cell activation and re-direction that combine good anticancer efficacy and low toxicity with favorable stability, manufacturability/producibility and structural homogeneity.

As a result of all the above efforts, the antigen-binding molecules and pharmaceutical compositions thereof can be used for targeting cells expressing CLDN6, for use in immunotherapy for treating various cancers, especially those associated with CLDN6 such as CLDN6-positive tumors.

More specifically, the present disclosure provides the following:

[1-1] A multispecific antigen-binding molecule comprising:
(i) a first antigen-binding moiety that is capable of binding to CD3 and CD137, but does not bind to CD3 and CD137 at the same time; and
(ii) a second antigen-binding moiety that is capable of binding to claudin-6 (CLDN6).
   [2-1] A multispecific antigen-binding molecule comprising:
(i) a first antigen-binding moiety that is capable of binding to CD3 and CD137, but does not bind to CD3 and CD137 at the same time; and
(ii) a second antigen-binding moiety that is capable of binding to claudin-6 (CLDN6);
wherein the first antigen-binding moiety comprises any one of (a1) to (a4) below:
(a1) a first antibody variable region comprising the complementarity determining region (CDR) 1 of SEQ ID NO: 9, the CDR 2 of SEQ ID NO: 15, and the CDR 3 of SEQ ID NO: 21, and a second antibody variable region comprising the CDR 1 of SEQ ID NO: 31, the CDR 2 of SEQ ID NO: 35, and the CDR 3 of SEQ ID NO: 39;
(a2) a first antibody variable region comprising the complementarity determining region (CDR) 1 of SEQ ID NO: 10, the CDR 2 of SEQ ID NO: 16, and the CDR 3 of SEQ ID NO: 22, and a second antibody variable region comprising the CDR 1 of SEQ ID NO: 31, the CDR 2 of SEQ ID NO: 35, and the CDR 3 of SEQ ID NO: 39;
(a3) a first antibody variable region comprising the complementarity determining region (CDR) 1 of SEQ ID NO: 11, the CDR 2 of SEQ ID NO: 17, and the CDR 3 of SEQ ID NO: 23, and a second antibody variable region comprising the CDR 1 of SEQ ID NO: 32, the CDR 2 of SEQ ID NO: 36, and the CDR 3 of SEQ ID NO: 40;
(a4) a first antibody variable region comprising the complementarity determining region (CDR) 1 of SEQ ID NO: 12, the CDR 2 of SEQ ID NO: 18, and the CDR 3 of SEQ ID NO: 24, and a second antibody variable region comprising the CDR 1 of SEQ ID NO: 32, the CDR 2 of SEQ ID NO: 36, and the CDR 3 of SEQ ID NO: 40.
   [2-2] The multispecific antigen-binding molecule of [2-1], wherein the second antigen-binding moiety comprises any one of (b1) to (b3) below:
(b1) a third antibody variable region comprising the complementarity determining region (CDR) 1 of SEQ ID NO: 8, the CDR 2 of SEQ ID NO: 14, and the CDR 3 of SEQ ID NO: 20, and a fourth antibody variable region comprising the CDR 1 of SEQ ID NO: 30, the CDR 2 of SEQ ID NO: 34, and the CDR 3 of SEQ ID NO: 38;
(b2) a third antibody variable region comprising the complementarity determining region (CDR) 1 of SEQ ID NO: 7, the CDR 2 of SEQ ID NO: 13, and the CDR 3 of SEQ ID NO: 19, and a fourth antibody variable region comprising the CDR 1 of SEQ ID NO: 29, the CDR 2 of SEQ ID NO: 33, and the CDR 3 of SEQ ID NO: 37;
(b3) a third antibody variable region comprising the complementarity determining region (CDR) 1 of SEQ ID NO: 29, the CDR 2 of SEQ ID NO: 33, and the CDR 3 of SEQ ID NO: 37, and a fourth antibody variable region comprising the CDR 1 of SEQ ID NO: 7, the CDR 2 of SEQ ID NO: 13, and the CDR 3 of SEQ ID NO: 19.
   [2-3] A multispecific antigen-binding molecule comprising:
(i) a first antigen-binding moiety that is capable of binding to CD3 and CD137, but does not bind to CD3 and CD137 at the same time; and
(ii) a second antigen-binding moiety that is capable of binding to claudin-6 (CLDN6);
wherein the second antigen-binding moiety comprises any one of (b1) to (b3) below:
(b1) a third antibody variable region comprising the complementarity determining region (CDR) 1 of SEQ ID NO: 8, the CDR 2 of SEQ ID NO: 14, and the CDR 3 of SEQ ID NO: 20, and a fourth antibody variable region comprising the CDR 1 of SEQ ID NO: 30, the CDR 2 of SEQ ID NO: 34, and the CDR 3 of SEQ ID NO: 38;
(b2) a third antibody variable region comprising the complementarity determining region (CDR) 1 of SEQ ID NO: 7, the CDR 2 of SEQ ID NO: 13, and the CDR 3 of SEQ ID NO: 19, and a fourth antibody variable region comprising the CDR 1 of SEQ ID NO: 29, the CDR 2 of SEQ ID NO: 33, and the CDR 3 of SEQ ID NO: 37;
(b3) a third antibody variable region comprising the complementarity determining region (CDR) 1 of SEQ ID NO: 29, the CDR 2 of SEQ ID NO: 33, and the CDR 3 of SEQ ID NO: 37, and a fourth antibody variable region comprising the CDR 1 of SEQ ID NO: 7, the CDR 2 of SEQ ID NO: 13, and the CDR 3 of SEQ ID NO: 19.
   [2-4] The multispecific antigen-binding molecule of any one of [2-1] to [2-3], wherein the antibody variable regions comprised in the first and/or second antigen-binding moiety comprise human antibody frameworks or humanized antibody frameworks.
   [2-5] A multispecific antigen-binding molecule comprising:
(i) a first antigen-binding moiety that is capable of binding to CD3 and CD137, but does not bind to CD3 and CD137 at the same time; and
(ii) a second antigen-binding moiety that is capable of binding to claudin-6 (CLDN6);
wherein the first antigen-binding moiety comprises any one of (c1) to (c4) below:
(c1) a first antibody variable region comprising the amino acid sequence of SEQ ID NO: 3, and a second antibody variable region comprising the amino acid sequence of SEQ ID NO: 27;
(c2) a first antibody variable region comprising the amino acid sequence of SEQ ID NO: 4, and a second antibody variable region comprising the amino acid sequence of SEQ ID NO: 27;
(c3) a first antibody variable region comprising the amino acid sequence of SEQ ID NO: 5, and a second antibody variable region comprising the amino acid sequence of SEQ ID NO: 28;
(c4) a first antibody variable region comprising the amino acid sequence of SEQ ID NO: 6, and a second antibody variable region comprising the amino acid sequence of SEQ ID NO: 28.
   [2-6] The multispecific antigen-binding molecule of [2-5], wherein the second antigen-binding moiety comprises any one of (d1) to (d3) below:
(d1) a third antibody variable region comprising the amino acid sequence of SEQ ID NO: 2, and a fourth antibody variable region comprising the amino acid sequence of SEQ ID NO: 26;
(d2) a third antibody variable region comprising the amino acid sequence of SEQ ID NO: 1, and a fourth antibody variable region comprising the amino acid sequence of SEQ ID NO: 25;
(d3) a third antibody variable region comprising the amino acid sequence of SEQ ID NO: 25, and a fourth antibody variable region comprising the amino acid sequence of SEQ ID NO: 1.
   [2-7] A multispecific antigen-binding molecule comprising:
(i) a first antigen-binding moiety that is capable of binding to CD3 and CD137, but does not bind to CD3 and CD137 at the same time; and
(ii) a second antigen-binding moiety that is capable of binding to claudin-6 (CLDN6);
wherein the second antigen-binding moiety comprises any one of (d1) to (d3) below:
(d1) a third antibody variable region comprising the amino acid sequence of SEQ ID NO: 2, and a fourth antibody variable region comprising the amino acid sequence of SEQ ID NO: 26;
(d2) a third antibody variable region comprising the amino acid sequence of SEQ ID NO: 1, and a fourth antibody variable region comprising the amino acid sequence of SEQ ID NO: 25;
(d3) a third antibody variable region comprising the amino acid sequence of SEQ ID NO: 25, and a fourth antibody variable region comprising the amino acid sequence of SEQ ID NO: 1.
   [2-8] A multispecific antigen-binding molecule comprising:
(i) a first antigen-binding moiety that binds to CD3; and
(ii) a second antigen-binding moiety that binds to claudin-6 (CLDN6);
wherein the first antigen-binding moiety comprises any one of (a1) to (a4) below:
(a1) a first antibody variable region comprising the complementarity determining region (CDR) 1 of SEQ ID NO: 9, the CDR 2 of SEQ ID NO: 15, and the CDR 3 of SEQ ID NO: 21, and a second antibody variable region comprising the CDR 1 of SEQ ID NO: 31, the CDR 2 of SEQ ID NO: 35, and the CDR 3 of SEQ ID NO: 39;
(a2) a first antibody variable region comprising the complementarity determining region (CDR) 1 of SEQ ID NO: 10, the CDR 2 of SEQ ID NO: 16, and the CDR 3 of SEQ ID NO: 22, and a second antibody variable region comprising the CDR 1 of SEQ ID NO: 31, the CDR 2 of SEQ ID NO: 35, and the CDR 3 of SEQ ID NO: 39;
(a3) a first antibody variable region comprising the complementarity determining region (CDR) 1 of SEQ ID NO: 11, the CDR 2 of SEQ ID NO: 17, and the CDR 3 of SEQ ID NO: 23, and a second antibody variable region comprising the CDR 1 of SEQ ID NO: 32, the CDR 2 of SEQ ID NO: 36, and the CDR 3 of SEQ ID NO: 40;
(a4) a first antibody variable region comprising the complementarity determining region (CDR) 1 of SEQ ID NO: 12, the CDR 2 of SEQ ID NO: 18, and the CDR 3 of SEQ ID NO: 24, and a second antibody variable region comprising the CDR 1 of SEQ ID NO: 32, the CDR 2 of SEQ ID NO: 36, and the CDR 3 of SEQ ID NO: 40.
   [2-9] The multispecific antigen-binding molecule of [2-8], wherein the second antigen-binding moiety comprises any one of (b1) to (b3) below:
(b1) a third antibody variable region comprising the complementarity determining region (CDR) 1 of SEQ ID NO: 8, the CDR 2 of SEQ ID NO: 14, and the CDR 3 of SEQ ID NO: 20, and a fourth antibody variable region comprising the CDR 1 of SEQ ID NO: 30, the CDR 2 of SEQ ID NO: 34, and the CDR 3 of SEQ ID NO: 38;
(b2) a third antibody variable region comprising the complementarity determining region (CDR) 1 of SEQ ID NO: 7, the CDR 2 of SEQ ID NO: 13, and the CDR 3 of SEQ ID NO: 19, and a fourth antibody variable region comprising the CDR 1 of SEQ ID NO: 29, the CDR 2 of SEQ ID NO: 33, and the CDR 3 of SEQ ID NO: 37;
(b3) a third antibody variable region comprising the complementarity determining region (CDR) 1 of SEQ ID NO: 29, the CDR 2 of SEQ ID NO: 33, and the CDR 3 of SEQ ID NO: 37, and a fourth antibody variable region comprising the CDR 1 of SEQ ID NO: 7, the CDR 2 of SEQ ID NO: 13, and the CDR 3 of SEQ ID NO: 19.
   [2-10] A multispecific antigen-binding molecule comprising:
(i) a first antigen-binding moiety that binds to CD3; and
(ii) a second antigen-binding moiety that binds to claudin-6 (CLDN6);
wherein the second antigen-binding moiety comprises any one of (b1) to (b3) below:
(b1) a third antibody variable region comprising the complementarity determining region (CDR) 1 of SEQ ID NO: 8, the CDR 2 of SEQ ID NO: 14, and the CDR 3 of SEQ ID NO: 20, and a fourth antibody variable region comprising the CDR 1 of SEQ ID NO: 30, the CDR 2 of SEQ ID NO: 34, and the CDR 3 of SEQ ID NO: 38;
(b2) a third antibody variable region comprising the complementarity determining region (CDR) 1 of SEQ ID NO: 7, the CDR 2 of SEQ ID NO: 13, and the CDR 3 of SEQ ID NO: 19, and a fourth antibody variable region comprising the CDR 1 of SEQ ID NO: 29, the CDR 2 of SEQ ID NO: 33, and the CDR 3 of SEQ ID NO: 37;
(b3) a third antibody variable region comprising the complementarity determining region (CDR) 1 of SEQ ID NO: 29, the CDR 2 of SEQ ID NO: 33, and the CDR 3 of SEQ ID NO: 37, and a fourth antibody variable region comprising the CDR 1 of SEQ ID NO: 7, the CDR 2 of SEQ ID NO: 13, and the CDR 3 of SEQ ID NO: 19.
   [2-11] The multispecific antigen-binding molecule of any one of [2-8] to [2-10], wherein the antibody variable regions comprised in the first and/or second antigen-binding moiety comprise human antibody frameworks or humanized antibody frameworks.
   [2-12] A multispecific antigen-binding molecule comprising:
(i) a first antigen-binding moiety that binds to CD3; and
(ii) a second antigen-binding moiety that binds to claudin-6 (CLDN6);
wherein the first antigen-binding moiety comprises any one of (c1) to (c4) below:
(c1) a first antibody variable region comprising the amino acid sequence of SEQ ID NO: 3, and a second antibody variable region comprising the amino acid sequence of SEQ ID NO: 27;
(c2) a first antibody variable region comprising the amino acid sequence of SEQ ID NO: 4, and a second antibody variable region comprising the amino acid sequence of SEQ ID NO: 27;
(c3) a first antibody variable region comprising the amino acid sequence of SEQ ID NO: 5, and a second antibody variable region comprising the amino acid sequence of SEQ ID NO: 28;
(c4) a first antibody variable region comprising the amino acid sequence of SEQ ID NO: 6, and a second antibody variable region comprising the amino acid sequence of SEQ ID NO: 28.
   [2-13] The multispecific antigen-binding molecule of [2-12], wherein the second antigen-binding moiety comprises any one of (d1) to (d3) below:
(d1) a third antibody variable region comprising the amino acid sequence of SEQ ID NO: 2, and a fourth antibody variable region comprising the amino acid sequence of SEQ ID NO: 26;
(d2) a third antibody variable region comprising the amino acid sequence of SEQ ID NO: 1, and a fourth antibody variable region comprising the amino acid sequence of SEQ ID NO: 25;
(d3) a third antibody variable region comprising the amino acid sequence of SEQ ID NO: 25, and a fourth antibody variable region comprising the amino acid sequence of SEQ ID NO: 1.
   [2-14] A multispecific antigen-binding molecule comprising:
(i) a first antigen-binding moiety that binds to CD3; and
(ii) a second antigen-binding moiety that binds to claudin-6 (CLDN6);
wherein the second antigen-binding moiety comprises any one of (d1) to (d3) below:
(d1) a third antibody variable region comprising the amino acid sequence of SEQ ID NO: 2, and a fourth antibody variable region comprising the amino acid sequence of SEQ ID NO: 26;
(d2) a third antibody variable region comprising the amino acid sequence of SEQ ID NO: 1, and a fourth antibody variable region comprising the amino acid sequence of SEQ ID NO: 25;
(d3) a third antibody variable region comprising the amino acid sequence of SEQ ID NO: 25, and a fourth antibody variable region comprising the amino acid sequence of SEQ ID NO: 1.
   [2-15] A multispecific antigen-binding molecule comprising:
(i) a first antigen-binding moiety that binds to CD137; and
(ii) a second antigen-binding moiety that binds to claudin-6 (CLDN6);
wherein the first antigen-binding moiety comprises any one of (a1) to (a4) below:
(a1) a first antibody variable region comprising the complementarity determining region (CDR) 1 of SEQ ID NO: 9, the CDR 2 of SEQ ID NO: 15, and the CDR 3 of SEQ ID NO: 21, and a second antibody variable region comprising the CDR 1 of SEQ ID NO: 31, the CDR 2 of SEQ ID NO: 35, and the CDR 3 of SEQ ID NO: 39;
(a2) a first antibody variable region comprising the complementarity determining region (CDR) 1 of SEQ ID NO: 10, the CDR 2 of SEQ ID NO: 16, and the CDR 3 of SEQ ID NO: 22, and a second antibody variable region comprising the CDR 1 of SEQ ID NO: 31, the CDR 2 of SEQ ID NO: 35, and the CDR 3 of SEQ ID NO: 39;
(a3) a first antibody variable region comprising the complementarity determining region (CDR) 1 of SEQ ID NO: 11, the CDR 2 of SEQ ID NO: 17, and the CDR 3 of SEQ ID NO: 23, and a second antibody variable region comprising the CDR 1 of SEQ ID NO: 32, the CDR 2 of SEQ ID NO: 36, and the CDR 3 of SEQ ID NO: 40;
(a4) a first antibody variable region comprising the complementarity determining region (CDR) 1 of SEQ ID NO: 12, the CDR 2 of SEQ ID NO: 18, and the CDR 3 of SEQ ID NO: 24, and a second antibody variable region comprising the CDR 1 of SEQ ID NO: 32, the CDR 2 of SEQ ID NO: 36, and the CDR 3 of SEQ ID NO: 40.
   [2-16] The multispecific antigen-binding molecule of [2-15], wherein the second antigen-binding moiety comprises any one of (b1) to (b3) below:
(b1) a third antibody variable region comprising the complementarity determining region (CDR) 1 of SEQ ID NO: 8, the CDR 2 of SEQ ID NO: 14, and the CDR 3 of SEQ ID NO: 20, and a fourth antibody variable region comprising the CDR 1 of SEQ ID NO: 30, the CDR 2 of SEQ ID NO: 34, and the CDR 3 of SEQ ID NO: 38;
(b2) a third antibody variable region comprising the complementarity determining region (CDR) 1 of SEQ ID NO: 7, the CDR 2 of SEQ ID NO: 13, and the CDR 3 of SEQ ID NO: 19, and a fourth antibody variable region comprising the CDR 1 of SEQ ID NO: 29, the CDR 2 of SEQ ID NO: 33, and the CDR 3 of SEQ ID NO: 37;
(b3) a third antibody variable region comprising the complementarity determining region (CDR) 1 of SEQ ID NO: 29, the CDR 2 of SEQ ID NO: 33, and the CDR 3 of SEQ ID NO: 37, and a fourth antibody variable region comprising the CDR 1 of SEQ ID NO: 7, the CDR 2 of SEQ ID NO: 13, and the CDR 3 of SEQ ID NO: 19.
   [2-17] A multispecific antigen-binding molecule comprising:
(i) a first antigen-binding moiety that binds to CD137; and
(ii) a second antigen-binding moiety that binds to claudin-6 (CLDN6);
wherein the second antigen-binding moiety comprises any one of (b1) to (b3) below:
(b1) a third antibody variable region comprising the complementarity determining region (CDR) 1 of SEQ ID NO: 8, the CDR 2 of SEQ ID NO: 14, and the CDR 3 of SEQ ID NO: 20, and a fourth antibody variable region comprising the CDR 1 of SEQ ID NO: 30, the CDR 2 of SEQ ID NO: 34, and the CDR 3 of SEQ ID NO: 38;
(b2) a third antibody variable region comprising the complementarity determining region (CDR) 1 of SEQ ID NO: 7, the CDR 2 of SEQ ID NO: 13, and the CDR 3 of SEQ ID NO: 19, and a fourth antibody variable region comprising the CDR 1 of SEQ ID NO: 29, the CDR 2 of SEQ ID NO: 33, and the CDR 3 of SEQ ID NO: 37;
(b3) a third antibody variable region comprising the complementarity determining region (CDR) 1 of SEQ ID NO: 29, the CDR 2 of SEQ ID NO: 33, and the CDR 3 of SEQ ID NO: 37, and a fourth antibody variable region comprising the CDR 1 of SEQ ID NO: 7, the CDR 2 of SEQ ID NO: 13, and the CDR 3 of SEQ ID NO: 19.
   [2-18] The multispecific antigen-binding molecule of any one of [2-15] to [2-17], wherein the antibody variable regions comprised in the first and/or second antigen-binding moiety comprise human antibody frameworks or humanized antibody frameworks.
   [2-19] A multispecific antigen-binding molecule comprising:
(i) a first antigen-binding moiety that binds to CD137; and
(ii) a second antigen-binding moiety that binds to claudin-6 (CLDN6);
wherein the first antigen-binding moiety comprises any one of (c1) to (c4) below:
(c1) a first antibody variable region comprising the amino acid sequence of SEQ ID NO: 3, and a second antibody variable region comprising the amino acid sequence of SEQ ID NO: 27;
(c2) a first antibody variable region comprising the amino acid sequence of SEQ ID NO: 4, and a second antibody variable region comprising the amino acid sequence of SEQ ID NO: 27;
(c3) a first antibody variable region comprising the amino acid sequence of SEQ ID NO: 5, and a second antibody variable region comprising the amino acid sequence of SEQ ID NO: 28;
(c4) a first antibody variable region comprising the amino acid sequence of SEQ ID NO: 6, and a second antibody variable region comprising the amino acid sequence of SEQ ID NO: 28.
   [2-20] The multispecific antigen-binding molecule of [2-19], wherein the second antigen-binding moiety comprises any one of (d1) to (d3) below:
(d1) a third antibody variable region comprising the amino acid sequence of SEQ ID NO: 2, and a fourth antibody variable region comprising the amino acid sequence of SEQ ID NO: 26;
(d2) a third antibody variable region comprising the amino acid sequence of SEQ ID NO: 1, and a fourth antibody variable region comprising the amino acid sequence of SEQ ID NO: 25;
(d3) a third antibody variable region comprising the amino acid sequence of SEQ ID NO: 25, and a fourth antibody variable region comprising the amino acid sequence of SEQ ID NO: 1.
   [2-21] A multispecific antigen-binding molecule comprising:
(i) a first antigen-binding moiety that binds to CD137; and
(ii) a second antigen-binding moiety that binds to claudin-6 (CLDN6);
wherein the second antigen-binding moiety comprises any one of (d1) to (d3) below:
(d1) a third antibody variable region comprising the amino acid sequence of SEQ ID NO: 2, and a fourth antibody variable region comprising the amino acid sequence of SEQ ID NO: 26;
(d2) a third antibody variable region comprising the amino acid sequence of SEQ ID NO: 1, and a fourth antibody variable region comprising the amino acid sequence of SEQ ID NO: 25;
(d3) a third antibody variable region comprising the amino acid sequence of SEQ ID NO: 25, and a fourth antibody variable region comprising the amino acid sequence of SEQ ID NO: 1.
   [2-22] A multispecific antigen-binding molecule comprising any one of (c1) to (c4) below:
(c1) a first antibody variable region comprising the amino acid sequence of SEQ ID NO: 3, and a second antibody variable region comprising the amino acid sequence of SEQ ID NO: 27;
(c2) a first antibody variable region comprising the amino acid sequence of SEQ ID NO: 4, and a second antibody variable region comprising the amino acid sequence of SEQ ID NO: 27;
(c3) a first antibody variable region comprising the amino acid sequence of SEQ ID NO: 5, and a second antibody variable region comprising the amino acid sequence of SEQ ID NO: 28;
(c4) a first antibody variable region comprising the amino acid sequence of SEQ ID NO: 6, and a second antibody variable region comprising the amino acid sequence of SEQ ID NO: 28.
   [2-23] The multispecific antigen-binding molecule of [2-22], wherein the multispecific antigen-binding molecule further comprises any one of (d1) to (d3) below:
(d1) a third antibody variable region comprising the amino acid sequence of SEQ ID NO: 2, and a fourth antibody variable region comprising the amino acid sequence of SEQ ID NO: 26;
(d2) a third antibody variable region comprising the amino acid sequence of SEQ ID NO: 1, and a fourth antibody variable region comprising the amino acid sequence of SEQ ID NO: 25;
(d3) a third antibody variable region comprising the amino acid sequence of SEQ ID NO: 25, and a fourth antibody variable region comprising the amino acid sequence of SEQ ID NO: 1.
   [2-24] A multispecific antigen-binding molecule comprising any one of (d1) to (d3) below:
(d1) a third antibody variable region comprising the amino acid sequence of SEQ ID NO: 2, and a fourth antibody variable region comprising the amino acid sequence of SEQ ID NO: 26;
(d2) a third antibody variable region comprising the amino acid sequence of SEQ ID NO: 1, and a fourth antibody variable region comprising the amino acid sequence of SEQ ID NO: 25;
(d3) a third antibody variable region comprising the amino acid sequence of SEQ ID NO: 25, and a fourth antibody variable region comprising the amino acid sequence of SEQ ID NO: 1.
   [2-25] A multispecific antigen-binding molecule comprising any one of (a1) to (a4) below:
(a1) a first antibody variable region comprising the complementarity determining region (CDR) 1 of SEQ ID NO: 9, the CDR 2 of SEQ ID NO: 15, and the CDR 3 of SEQ ID NO: 21, and a second antibody variable region comprising the CDR 1 of SEQ ID NO: 31, the CDR 2 of SEQ ID NO: 35, and the CDR 3 of SEQ ID NO: 39;
(a2) a first antibody variable region comprising the complementarity determining region (CDR) 1 of SEQ ID NO: 10, the CDR 2 of SEQ ID NO: 16, and the CDR 3 of SEQ ID NO: 22, and a second antibody variable region comprising the CDR 1 of SEQ ID NO: 31, the CDR 2 of SEQ ID NO: 35, and the CDR 3 of SEQ ID NO: 39;
(a3) a first antibody variable region comprising the complementarity determining region (CDR) 1 of SEQ ID NO: 11, the CDR 2 of SEQ ID NO: 17, and the CDR 3 of SEQ ID NO: 23, and a second antibody variable region comprising the CDR 1 of SEQ ID NO: 32, the CDR 2 of SEQ ID NO: 36, and the CDR 3 of SEQ ID NO: 40;
(a4) a first antibody variable region comprising the complementarity determining region (CDR) 1 of SEQ ID NO: 12, the CDR 2 of SEQ ID NO: 18, and the CDR 3 of SEQ ID NO: 24, and a second antibody variable region comprising the CDR 1 of SEQ ID NO: 32, the CDR 2 of SEQ ID NO: 36, and the CDR 3 of SEQ ID NO: 40.
   [2-26] The multispecific antigen-binding molecule of [2-25], wherein the multispecific antigen-binding molecule further comprises any one of (b1) to (b3) below:
(b1) a third antibody variable region comprising the complementarity determining region (CDR) 1 of SEQ ID NO: 8, the CDR 2 of SEQ ID NO: 14, and the CDR 3 of SEQ ID NO: 20, and a fourth antibody variable region comprising the CDR 1 of SEQ ID NO: 30, the CDR 2 of SEQ ID NO: 34, and the CDR 3 of SEQ ID NO: 38;
(b2) a third antibody variable region comprising the complementarity determining region (CDR) 1 of SEQ ID NO: 7, the CDR 2 of SEQ ID NO: 13, and the CDR 3 of SEQ ID NO: 19, and a fourth antibody variable region comprising the CDR 1 of SEQ ID NO: 29, the CDR 2 of SEQ ID NO: 33, and the CDR 3 of SEQ ID NO: 37;
(b3) a third antibody variable region comprising the complementarity determining region (CDR) 1 of SEQ ID NO: 29, the CDR 2 of SEQ ID NO: 33, and the CDR 3 of SEQ ID NO: 37, and a fourth antibody variable region comprising the CDR 1 of SEQ ID NO: 7, the CDR 2 of SEQ ID NO: 13, and the CDR 3 of SEQ ID NO: 19.
   [2-27] A multispecific antigen-binding molecule comprising any one of (b1) to (b3) below:
(b1) a third antibody variable region comprising the complementarity determining region (CDR) 1 of SEQ ID NO: 8, the CDR 2 of SEQ ID NO: 14, and the CDR 3 of SEQ ID NO: 20, and a fourth antibody variable region comprising the CDR 1 of SEQ ID NO: 30, the CDR 2 of SEQ ID NO: 34, and the CDR 3 of SEQ ID NO: 38;
(b2) a third antibody variable region comprising the complementarity determining region (CDR) 1 of SEQ ID NO: 7, the CDR 2 of SEQ ID NO: 13, and the CDR 3 of SEQ ID NO: 19, and a fourth antibody variable region comprising the CDR 1 of SEQ ID NO: 29, the CDR 2 of SEQ ID NO: 33, and the CDR 3 of SEQ ID NO: 37;
(b3) a third antibody variable region comprising the complementarity determining region (CDR) 1 of SEQ ID NO: 29, the CDR 2 of SEQ ID NO: 33, and the CDR 3 of SEQ ID NO: 37, and a fourth antibody variable region comprising the CDR 1 of SEQ ID NO: 7, the CDR 2 of SEQ ID NO: 13, and the CDR 3 of SEQ ID NO: 19.
   [3-1] The multispecific antigen-binding molecule of any one of [1-1] to [2-27], which further comprises:
(iii) an Fc domain which exhibits reduced binding affinity to human Fc gamma receptor, as compared to a native human IgG1 Fc domain.
   [3-2] The multispecific antigen-binding molecule of [3-1], wherein the Fc domain is composed of a first Fc-region subunit and a second Fc-region subunit that are capable of stable association.
   [3-3] The multispecific antigen-binding molecule of [3-2], wherein the Fc domain comprises (e1) or (e2) below:
(e1) the first Fc-region subunit comprising Cys at position 349, Ser at position 366, Ala at position 368 and Val at position 407, and the second Fc-region comprising Cys at position 354 and Trp at position 366;
(e2) the first Fc-region subunit comprising Glu at position 439, and the second Fc-region comprising Lys at position 356;
wherein the amino acid positions are numbered according to EU index.
   [3-4] The multispecific antigen-binding molecule of [3-2] or [3-3], wherein the first and/or the second Fc-region subunit comprises (f1) or (f2) below:
(f1) Ala at position 234 and Ala at position 235;
(f2) Ala at position 234, Ala at position 235 and Ala at position 297;
wherein the amino acid positions are numbered according to EU index.
   [3-5] The multispecific antigen-binding molecule of any one of [3-2] to [3-4], wherein the Fc domain further exhibits stronger FcRn binding affinity to human FcRn, as compared to a native human IgG1 Fc domain.
   [3-6] The multispecific antigen-binding molecule of [3-5], wherein the first and/or the second Fc-region subunit comprises Leu at position 428, Ala at position 434, Arg at position 438, and Glu at position 440,
wherein the amino acid positions are numbered according to EU index.
   [3-7] The multispecific antigen-binding molecule of any one of [2-1] to [2-27], wherein the first antibody variable region of the first antigen-binding moiety is fused with a first heavy chain constant region, the second antibody variable region of the first antigen-binding moiety is fused with a first light chain constant region, the third antibody variable region of the second antigen-binding moiety is fused with a second heavy chain constant region, the fourth antibody variable region of the second antigen-binding moiety is fused with a second light chain constant region,
wherein the constant regions are any one of (g1) to (g7) below:
(g1) a first heavy chain constant region comprising the amino acid sequence of SEQ ID NO: 74, a first light chain constant region comprising the amino acid sequence of SEQ ID NO: 87, a second heavy chain constant region comprising the amino acid sequence of SEQ ID NO: 73, and a second light chain constant region comprising the amino acid sequence of SEQ ID NO: 88;
(g2) a first heavy chain constant region comprising the amino acid sequence of SEQ ID NO: 74, a first light chain constant region comprising the amino acid sequence of SEQ ID NO: 85, a second heavy chain constant region comprising the amino acid sequence of SEQ ID NO: 81, and a second light chain constant region comprising the amino acid sequence of SEQ ID NO: 86;
(g3) a first heavy chain constant region comprising the amino acid sequence of SEQ ID NO: 79, a first light chain constant region comprising the amino acid sequence of SEQ ID NO: 72, a second heavy chain constant region comprising the amino acid sequence of SEQ ID NO: 80, and a second light chain constant region comprising the amino acid sequence of SEQ ID NO: 89;
(g4) a first heavy chain constant region comprising the amino acid sequence of SEQ ID NO: 83, a first light chain constant region comprising the amino acid sequence of SEQ ID NO: 87, a second heavy chain constant region comprising the amino acid sequence of SEQ ID NO: 82, and a second light chain constant region comprising the amino acid sequence of SEQ ID NO: 88;
(g5) a first heavy chain constant region comprising the amino acid sequence of SEQ ID NO: 83, a first light chain constant region comprising the amino acid sequence of SEQ ID NO: 85, a second heavy chain constant region comprising the amino acid sequence of SEQ ID NO: 84, and a second light chain constant region comprising the amino acid sequence of SEQ ID NO: 86;
(g6) a first heavy chain constant region comprising the amino acid sequence of SEQ ID NO: 77, a first light chain constant region comprising the amino acid sequence of SEQ ID NO: 72, a second heavy chain constant region comprising the amino acid sequence of SEQ ID NO: 78, and a second light chain constant region comprising the amino acid sequence of SEQ ID NO: 89;
(g7) a first heavy chain constant region comprising the amino acid sequence of SEQ ID NO: 75, a first light chain constant region comprising the amino acid sequence of SEQ ID NO: 72, a second heavy chain constant region comprising the amino acid sequence of SEQ ID NO: 76, and a second light chain constant region comprising the amino acid sequence of SEQ ID NO: 89.
   [4-1] A multispecific antigen-binding molecule comprising four polypeptide chains, wherein the four polypeptide chains are any one of (h01) to (h18) below:
(h01) a heavy chain comprising the amino acid sequence of SEQ ID NO: 41 (chain 1) and a light chain comprising the amino acid sequence of SEQ ID NO: 50 (chain 2), and a heavy chain comprising the amino acid sequence of SEQ ID NO: 54 (chain 3) and a light chain comprising the amino acid sequence of SEQ ID NO: 68 (chain 4);
(h02) a heavy chain comprising the amino acid sequence of SEQ ID NO: 41 (chain 1) and a light chain comprising the amino acid sequence of SEQ ID NO: 50 (chain 2), and a heavy chain comprising the amino acid sequence of SEQ ID NO: 55 (chain 3) and a light chain comprising the amino acid sequence of SEQ ID NO: 68 (chain 4);
(h03) a heavy chain comprising the amino acid sequence of SEQ ID NO: 42 (chain 1) and a light chain comprising the amino acid sequence of SEQ ID NO: 51 (chain 2), and a heavy chain comprising the amino acid sequence of SEQ ID NO: 56 (chain 3) and a light chain comprising the amino acid sequence of SEQ ID NO: 69 (chain 4);
(h04) a heavy chain comprising the amino acid sequence of SEQ ID NO: 42 (chain 1) and a light chain comprising the amino acid sequence of SEQ ID NO: 51 (chain 2), and a heavy chain comprising the amino acid sequence of SEQ ID NO: 57 (chain 3) and a light chain comprising the amino acid sequence of SEQ ID NO: 69 (chain 4);
(h05) a heavy chain comprising the amino acid sequence of SEQ ID NO: 44 (chain 1) and a light chain comprising the amino acid sequence of SEQ ID NO: 52 (chain 2), and a heavy chain comprising the amino acid sequence of SEQ ID NO: 60 (chain 3) and a light chain comprising the amino acid sequence of SEQ ID NO: 70 (chain 4);
(h06) a heavy chain comprising the amino acid sequence of SEQ ID NO: 44 (chain 1) and a light chain comprising the amino acid sequence of SEQ ID NO: 52 (chain 2), and a heavy chain comprising the amino acid sequence of SEQ ID NO: 61 (chain 3) and a light chain comprising the amino acid sequence of SEQ ID NO: 70 (chain 4);
(h07) a heavy chain comprising the amino acid sequence of SEQ ID NO: 45 (chain 1) and a light chain comprising the amino acid sequence of SEQ ID NO: 50 (chain 2), and a heavy chain comprising the amino acid sequence of SEQ ID NO: 62 (chain 3) and a light chain comprising the amino acid sequence of SEQ ID NO: 68 (chain 4);
(h08) a heavy chain comprising the amino acid sequence of SEQ ID NO: 45 (chain 1) and a light chain comprising the amino acid sequence of SEQ ID NO: 50 (chain 2), and a heavy chain comprising the amino acid sequence of SEQ ID NO: 63 (chain 3) and a light chain comprising the amino acid sequence of SEQ ID NO: 68 (chain 4);
(h09) a heavy chain comprising the amino acid sequence of SEQ ID NO: 46 (chain 1) and a light chain comprising the amino acid sequence of SEQ ID NO: 51 (chain 2), and a heavy chain comprising the amino acid sequence of SEQ ID NO: 64 (chain 3) and a light chain comprising the amino acid sequence of SEQ ID NO: 69 (chain 4);
(h10) a heavy chain comprising the amino acid sequence of SEQ ID NO: 46 (chain 1) and a light chain comprising the amino acid sequence of SEQ ID NO: 51 (chain 2), and a heavy chain comprising the amino acid sequence of SEQ ID NO: 65 (chain 3) and a light chain comprising the amino acid sequence of SEQ ID NO: 69 (chain 4);
(h11) a heavy chain comprising the amino acid sequence of SEQ ID NO: 47 (chain 1) and a light chain comprising the amino acid sequence of SEQ ID NO: 52 (chain 2), and a heavy chain comprising the amino acid sequence of SEQ ID NO: 66 (chain 3) and a light chain comprising the amino acid sequence of SEQ ID NO: 70 (chain 4);
(h12) a heavy chain comprising the amino acid sequence of SEQ ID NO: 47 (chain 1) and a light chain comprising the amino acid sequence of SEQ ID NO: 52 (chain 2), and a heavy chain comprising the amino acid sequence of SEQ ID NO: 67 (chain 3) and a light chain comprising the amino acid sequence of SEQ ID NO: 70 (chain 4);
(h13) a heavy chain comprising the amino acid sequence of SEQ ID NO: 48 (chain 1) and a light chain comprising the amino acid sequence of SEQ ID NO: 53 (chain 2), and a heavy chain comprising the amino acid sequence of SEQ ID NO: 56 (chain 3) and a light chain comprising the amino acid sequence of SEQ ID NO: 71 (chain 4);
(h14) a heavy chain comprising the amino acid sequence of SEQ ID NO: 48 (chain 1) and a light chain comprising the amino acid sequence of SEQ ID NO: 53 (chain 2), and a heavy chain comprising the amino acid sequence of SEQ ID NO: 57 (chain 3) and a light chain comprising the amino acid sequence of SEQ ID NO: 71 (chain 4);
(h15) a heavy chain comprising the amino acid sequence of SEQ ID NO: 49 (chain 1) and a light chain comprising the amino acid sequence of SEQ ID NO: 53 (chain 2), and a heavy chain comprising the amino acid sequence of SEQ ID NO: 64 (chain 3) and a light chain comprising the amino acid sequence of SEQ ID NO: 71 (chain 4);
(h16) a heavy chain comprising the amino acid sequence of SEQ ID NO: 49 (chain 1) and a light chain comprising the amino acid sequence of SEQ ID NO: 53 (chain 2), and a heavy chain comprising the amino acid sequence of SEQ ID NO: 65 (chain 3) and a light chain comprising the amino acid sequence of SEQ ID NO: 71 (chain 4);
(h17) a heavy chain comprising the amino acid sequence of SEQ ID NO: 43 (chain 1) and a light chain comprising the amino acid sequence of SEQ ID NO: 52 (chain 2), and a heavy chain comprising the amino acid sequence of SEQ ID NO: 58 (chain 3) and a light chain comprising the amino acid sequence of SEQ ID NO: 70 (chain 4); and
(h18) a heavy chain comprising the amino acid sequence of SEQ ID NO: 43 (chain 1) and a light chain comprising the amino acid sequence of SEQ ID NO: 52 (chain 2), and a heavy chain comprising the amino acid sequence of SEQ ID NO: 59 (chain 3) and a light chain comprising the amino acid sequence of SEQ ID NO: 70 (chain 4).
   [4-2] The multispecific antigen-binding molecule of [4-1], wherein
(i) an antibody variable region comprised in chain 3 and an antibody variable region comprised in chain 4 form a first antigen-binding moiety that is capable of binding to CD3 and CD137, but does not bind to CD3 and CD137 at the same time;
(ii) an antibody variable region comprised in chain 1 and an antibody variable region comprised in chain 2 form a second antigen-binding moiety that is capable of binding to claudin-6 (CLDN6);
(iii) an antibody Fc region subunit comprised in chain 1 and an antibody Fc region subunit comprised in chain 3 forms an Fc domain.
   [4-3] The multispecific antigen-binding molecule of [4-1], wherein
(i) an antibody variable region comprised in chain 3 and an antibody variable region comprised in chain 4 form a first antigen-binding moiety that binds to CD3;
(ii) an antibody variable region comprised in chain 1 and an antibody variable region comprised in chain 2 form a second antigen-binding moiety that binds to claudin-6 (CLDN6);
(iii) an antibody Fc region subunit comprised in chain 1 and an antibody Fc region subunit comprised in chain 3 forms an Fc domain.
   [4-4] The multispecific antigen-binding molecule of [4-1], wherein
(i) an antibody variable region comprised in chain 3 and an antibody variable region comprised in chain 4 form a first antigen-binding moiety that binds to CD137;
(ii) an antibody variable region comprised in chain 1 and an antibody variable region comprised in chain 2 form a second antigen-binding moiety that binds to claudin-6 (CLDN6);
(iii) an antibody Fc region subunit comprised in chain 1 and an antibody Fc region subunit comprised in chain 3 forms an Fc domain.
   [5-1] The multispecific antigen-binding molecule of any one of [1-1] to [3-7] or [4-4], wherein the second antigen-binding moiety is capable of binding to human CLDN6.
   [5-2] The multispecific antigen-binding molecule of any one of [1-1] to [3-7] or [4-4], wherein the second antigen-binding moiety is capable of binding to human CLDN6 as defined in SEQ ID NO: 196 or 197.
   [5-3] The multispecific antigen-binding molecule of any one of [1-1] to [3-7] or [4-4], wherein the second antigen-binding moiety does not substantially bind to human CLDN9.
   [5-4] The multispecific antigen-binding molecule of any one of [1-1] to [3-7] or [4-4], wherein the second antigen-binding moiety does not substantially bind to human CLDN4.
   [5-5] The multispecific antigen-binding molecule of any one of [1-1] to [3-7] or [4-4], wherein the second antigen-binding moiety does not substantially bind to human CLDN3.
   [5-6] The multispecific antigen-binding molecule of any one of [1-1] to [3-7] or [4-4], wherein the second antigen-binding moiety does not substantially bind to a CLDN6 mutant as defined in SEQ ID NO: 205.
   [6-1] An isolated nucleic acid encoding the multispecific antigen-binding molecule of any one of [1-1] to [5-6].
   [6-2] A host cell comprising the nucleic acid of [6-1].
   [6-3] A method of producing a multispecific antigen-binding molecule comprising culturing the host cell of [6-2] so that the multispecific antigen-binding molecule is produced.
   [6-4] The method of [6-3], further comprising recovering the multispecific antigen-binding molecule from the culture of the host cell.
   [7-1] A pharmaceutical composition comprising the multispecific antigen-binding molecule of any one of [1-1] to [5-6], and a pharmaceutically acceptable carrier.
   [7-2] The pharmaceutical composition of [7-1], which is a pharmaceutical composition used for treatment and/or prevention of cancer.
   [7-3] The pharmaceutical composition of [7-2], wherein the cancer comprises CLDN6 expressing cells.
   [7-4] The pharmaceutical composition of [7-2] or [7-3], wherein the cancer is ovarian cancer, non-small cell lung cancer, gastric cancer, liver cancer, endometrial cancer, or germ cell tumor.
   [7-5] Use of the multispecific antigen-binding molecule of any one of [1-1] to [5-6] in the manufacture of a medicament.
   [7-6] Use of the multispecific antigen-binding molecule of any one of [1-1] to [5-6] in the manufacture of a medicament for treatment and/or prevention of cancer.
   [7-7] Use of the multispecific antigen-binding molecule of any one of [1-1] to [5-6] in the manufacture of a medicament for treatment and/or prevention of cancer that comprises CLDN6 expressing cells.
   [7-8] Use of the multispecific antigen-binding molecule of any one of [1-1] to [5-6] in the manufacture of a medicament for treatment and/or prevention of ovarian cancer, non-small cell lung cancer, gastric cancer, liver cancer, endometrial cancer, or germ cell tumor.
   [7-9] A method of treating an individual having cancer comprising administering to the individual an effective amount of the multispecific antigen-binding molecule of any one of [1-1] to [5-6].
   [7-10] The method of [7-9], wherein the cancer comprises CLDN6 expressing cells.
   [7-11] The method of [7-9] or [7-10], wherein the cancer is ovarian cancer, non-small cell lung cancer, gastric cancer, liver cancer, endometrial cancer, or germ cell tumor.
   [7-12] A kit for use in the treatment and/or prevention of cancer, which comprises at least the multispecific antigen-binding molecule of any one of [1-1] to [5-6], and instructions for use.
   [7-13] The kit of [7-12], wherein the cancer comprises CLDN6 expressing cells.
   [7-14] The kit of [7-12], wherein the cancer is ovarian cancer, non-small cell lung cancer, gastric cancer, liver cancer, endometrial cancer, or germ cell tumor.

### BRIEF DESCRIPTION OF DRAWINGS

FIG. 1 shows the epitope of the H0868L0581 Fab contact region on the CD137. Epitope mapping in the CD137 amino acid sequence (black: closer than 3.0 angstrom, stripes: closer than 4.5 angstrom from H0868L0581).

FIG. 2 shows the epitope of the H0868L0581 Fab contact region on the CD137. Epitope mapping in the crystal structure (dark gray spheres: closer than 3.0 angstrom, light gray sticks: closer than 4.5 angstrom from H0868L0581).

FIG. 3 illustrates various antibody formats. Annotation of each Fv region for Table 4 and naming rule for Table 4, Table 5 and Table 6. Diagram depicting (a) 1+1 Bispecific antibodies by utilizing FAST-Ig; (b) 1+1 Bispecific antibodies by utilizing CrossMab technology.

FIG. 4 shows binding activity of anti-CLDN6/CD3 bispecific antibodies (CS2961, and 6PHU3/TR01) to human CLDN family proteins (CLDN3, CLDN4, CLDN6, and CLDN9). Binding activity of anti-CLDN6/CD3 bispecific antibodies to BaF3 transfectants (hCLDN6/BaF, hCLDN3/BaF, hCLDN4/BaF, and hCLDN9/BaF) was examined by flow cytometer in a concentration of 15 micro g/ml, and plotted as histogram. KLH/TR01 was used as a negative control.

FIG. 5 shows T-cell-dependent cell cytotoxicity evaluation by LDH assay.

FIG. 6 shows amino acid sequence alignment of human CLDN9 and human CLDN6. Human CLDN9 and human CLDN6 comprise almost the same sequence in extracellular domain 1, except the N-terminus residue (Met/Leu at position 29). Two amino acids in extracellular domain 2 are different between human CLDN9 and human CLDN6 (Arg/Leu at position 145 and Gln/Leu at position 156).

FIG. 7 shows T-cell-dependent cell cytotoxicity evaluation results of the antibody (PPU4135) towards various cancer cell lines by LDH assay.

FIG. 8 shows real-time cell growth inhibition assay results of the antibodies (CS3348, PPU4135 and PPU4136) towards various cancer cell lines by xCELLigence assay.

FIG. 9 shows T cell activation through CD3 binding by the antibodies (CS3348, PPU4135, PPU4136, PPU4137 and PPU4138), under co-culture with CLDN6 expressing human cell lines (OVCAR3 and NCI-H1435) and CLDN6 negative cell line (5637). KLH/TR01 was used as a negative control.

FIG. 10 shows NF kappa B activation through CD137 binding by the antibodies (CS3348, PPU4134, PPU4135, PPU4136, PPU4137 and PPU4138), under co-culture with CLDN6 expressing human cell lines (OVCAR3 and NCI-H1435) and CLDN6 negative cell line (5637). KLH/TR01 was used as a negative control.

FIG. 11 shows in vivo anti-tumor efficacy of the antibodies (CS3348, PPU4134, PPU4135, PPU4136, PPU4137 and PPU4138) at a dose of 1 mg/kg using NCI-H1435/HuNOG mice model.

FIG. 12 shows in vivo anti-tumor efficacy of the antibodies (CS3348 and PPU4135) at doses of 0.05 mg/kg and 0.2 mg/kg using OV-90/HuNOG mice model.

FIG. 13 shows AST, ALT, GLDH (hepatic enzymes), ALP, TBIL, GGT, TBA (hepatobiliary damage parameters), and CRP (inflammatory marker) level changes mediated by CS3348 or PPU4135 administration.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

The definitions and detailed description below are provided to facilitate understanding of the present disclosure illustrated herein.

### Definitions

### Amino Acids

Herein, amino acids are described by one- or three-letter codes or both, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V.

### Alteration of Amino Acids

For amino acid alteration (also described as “amino acid substitution” or “amino acid mutation” within this description) in the amino acid sequence of an antigen-binding molecule, known methods such as site-directed mutagenesis methods (Kunkel et al. (Proc. Natl. Acad. Sci. USA (1985) 82, 488-492)) and overlap extension PCR may be appropriately employed. Furthermore, several known methods may also be employed as amino acid alteration methods for substitution to non-natural amino acids (Annu Rev. Biophys. Biomol. Struct. (2006) 35, 225-249; and Proc. Natl. Acad. Sci. U.S.A. (2003) 100 (11), 6353-6357). For example, it is suitable to use a cell-free translation system (Clover Direct (Protein Express)) containing a tRNA which has a non-natural amino acid bound to a complementary amber suppressor tRNA of one of the stop codons, the UAG codon (amber codon).

In the present specification, the meaning of the term “and/or” when describing the site of amino acid alteration includes every combination where “and” and “or” are suitably combined. Specifically, for example, “the amino acids at positions 33, 55, and/or 96 are substituted” includes the following variation of amino acid alterations: amino acid(s) at (a) position 33, (b) position 55, (c) position 96, (d) positions 33 and 55, (e) positions 33 and 96, (f) positions 55 and 96, and (g) positions 33, 55, and 96.

Furthermore, herein, as an expression showing alteration of amino acids, an expression that shows before and after a number indicating a specific position, one-letter or three-letter codes for amino acids before and after alteration, respectively, may be used appropriately. For example, the alteration N100bL or Asn100bLeu used when substituting an amino acid contained in an antibody variable region indicates substitution of Asn at position 100b (according to Kabat numbering) with Leu. That is, the number shows the amino acid position according to Kabat numbering, the one-letter or three-letter amino-acid code written before the number shows the amino acid before substitution, and the one-letter or three-letter amino-acid code written after the number shows the amino acid after substitution. Similarly the alteration P238D or Pro238Asp used when substituting an amino acid of the Fc region contained in an antibody constant region indicates substitution of Pro at position 238 (according to EU numbering) with Asp. That is, the number shows the amino acid position according to EU numbering, the one-letter or three-letter amino-acid code written before the number shows the amino acid before substitution, and the one-letter or three-letter amino-acid code written after the number shows the amino acid after substitution.

### Polypeptides

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide as described herein may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

### Percent (%) Amino Acid Sequence Identity

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Recombinant Methods and Compositions

Antibodies and antigen-binding molecules may be produced using recombinant methods and compositions, e.g., as described in U.S. Pat. No. 4,816,567. In one embodiment, isolated nucleic acid encoding an antibody as described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making the multispecific antigen-binding molecule of the present disclosure is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody described herein, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Recombinant production of an antigen-binding molecule described herein could be done with methods similar to those described above, by using a host cell comprises (e.g., has been transformed with) one or plural vectors comprising nucleic acid that encodes an amino acid sequence comprising the whole antigen-binding molecule or part of the antigen-binding molecule.

### Antigen-Binding Molecules and Multispecific Antigen-Binding Molecules

The term “antigen-binding molecule”, as used herein, refers to any molecule that comprises an antigen-binding site or any molecule that has binding activity to an antigen, and may further refer to molecules such as a peptide or protein having a length of about five amino acids or more. The peptide and protein are not limited to those derived from a living organism, and for example, they may be a polypeptide produced from an artificially designed sequence. They may also be any of a naturally-occurring polypeptide, synthetic polypeptide, recombinant polypeptide, and such. Scaffold molecules comprising known stable conformational structure such as alpha/beta barrel as scaffold, and in which part of the molecule is made into antigen-binding site, is also one embodiment of the antigen binding molecule described herein.

“Multispecific antigen-binding molecules” refers to antigen-binding molecules that bind specifically to more than one antigen. The term “bispecific” means that the antigen binding molecule is able to specifically bind to at least two distinct antigenic determinants. The term “trispecific” means that the antigen binding molecule is able to specifically bind to at least three distinct antigenic determinants.

In certain embodiments, the multispecific antigen-binding molecule of the present application is a trispecific antigen-binding molecule, i.e. capable of specifically binding to three different antigens-capable of binding to either one of CD3 or CD137 but does not bind to both antigens simultaneously, and is capable of specifically binding to CLDN6.

In one aspect, the present disclosure provides a multispecific antigen-binding molecule comprising:
(i) a first antigen binding moiety capable of binding to CD3 and CD137, but does not bind to CD3 and CD137 at the same time; and
(ii) a second antigen-binding moiety that is capable of binding to Claudin-6 (CLDN6), preferably human CLDN6.

In one aspect, the present disclosure provides a multispecific antigen-binding molecule further comprising
(iii) an Fc domain which exhibits reduced binding affinity to human Fc gamma receptor, as compared to a native human IgG1 Fc domain.

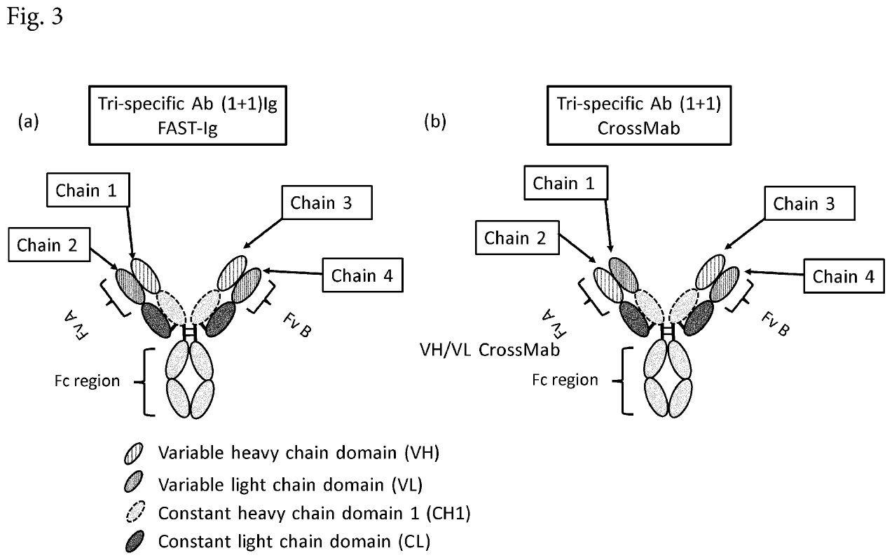

The components of the multispecific antigen-binding molecules of the present disclosure can be fused to each other in a variety of configurations. Exemplary configurations are depicted in FIG. 3. In particular embodiments, the multispecific antigen-binding molecules comprise an Fc domain composed of a first Fc-region subunit and a second Fc-region subunit that are capable of stable association.

According to any of the above embodiments, components of the multispecific antigen-binding molecules (e.g. antigen binding moiety, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G4S)n, (SG4)n, (G4S)n or G4(SG4)n peptide linkers, wherein n is generally a number between 1 and 10, typically between 2 and 4.

In one aspect, the present disclosure provides a multispecific antigen-binding molecule comprising:
(i) a first antigen-binding moiety that is capable of binding to CD3 and CD137, but does not bind to CD3 and CD137 at the same time; and
(ii) a second antigen-binding moiety that is capable of binding to claudin-6 (CLDN6);
   wherein the first antibody variable region of the first antigen-binding moiety is fused with a first heavy chain constant region, the second antibody variable region of the first antigen-binding moiety is fused with a first light chain constant region, the third antibody variable region of the second antigen-binding moiety is fused with a second heavy chain constant region, the fourth antibody variable region of the second antigen-binding moiety is fused with a second light chain constant region.

In one aspect, the present disclosure provides a multispecific antigen-binding molecule comprising:
(i) a first antigen-binding moiety that is capable of binding to CD3 and CD137, but does not bind to CD3 and CD137 at the same time; and
(ii) a second antigen-binding moiety that is capable of binding to claudin-6 (CLDN6);
   wherein the first antibody variable region of the first antigen-binding moiety is fused with a first heavy chain constant region, the second antibody variable region of the first antigen-binding moiety is fused with a first light chain constant region, the third antibody variable region of the second antigen-binding moiety is fused with a second heavy chain constant region, the fourth antibody variable region of the second antigen-binding moiety is fused with a second light chain constant region, wherein the constant regions are any one of (g1) to (g7) below:
(g1) a first heavy chain constant region comprising the amino acid sequence of SEQ ID NO: 74, a first light chain constant region comprising the amino acid sequence of SEQ ID NO: 87, a second heavy chain constant region comprising the amino acid sequence of SEQ ID NO: 73, and a second light chain constant region comprising the amino acid sequence of SEQ ID NO: 88;
(g2) a first heavy chain constant region comprising the amino acid sequence of SEQ ID NO: 74, a first light chain constant region comprising the amino acid sequence of SEQ ID NO: 85, a second heavy chain constant region comprising the amino acid sequence of SEQ ID NO: 81, and a second light chain constant region comprising the amino acid sequence of SEQ ID NO: 86;
(g3) a first heavy chain constant region comprising the amino acid sequence of SEQ ID NO: 79, a first light chain constant region comprising the amino acid sequence of SEQ ID NO: 72, a second heavy chain constant region comprising the amino acid sequence of SEQ ID NO: 80, and a second light chain constant region comprising the amino acid sequence of SEQ ID NO: 89;
(g4) a first heavy chain constant region comprising the amino acid sequence of SEQ ID NO: 83, a first light chain constant region comprising the amino acid sequence of SEQ ID NO: 87, a second heavy chain constant region comprising the amino acid sequence of SEQ ID NO: 82, and a second light chain constant region comprising the amino acid sequence of SEQ ID NO: 88;
(g5) a first heavy chain constant region comprising the amino acid sequence of SEQ ID NO: 83, a first light chain constant region comprising the amino acid sequence of SEQ ID NO: 85, a second heavy chain constant region comprising the amino acid sequence of SEQ ID NO: 84, and a second light chain constant region comprising the amino acid sequence of SEQ ID NO: 86;
(g6) a first heavy chain constant region comprising the amino acid sequence of SEQ ID NO: 77, a first light chain constant region comprising the amino acid sequence of SEQ ID NO: 72, a second heavy chain constant region comprising the amino acid sequence of SEQ ID NO: 78, and a second light chain constant region comprising the amino acid sequence of SEQ ID NO: 89;
(g7) a first heavy chain constant region comprising the amino acid sequence of SEQ ID NO: 75, a first light chain constant region comprising the amino acid sequence of SEQ ID NO: 72, a second heavy chain constant region comprising the amino acid sequence of SEQ ID NO: 76, and a second light chain constant region comprising the amino acid sequence of SEQ ID NO: 89.

In one aspect, the present disclosure provides a multispecific antigen-binding molecule comprising four polypeptide chains, wherein the four polypeptide chains are any one of (h01) to (h18) below:
(h01) a heavy chain comprising the amino acid sequence of SEQ ID NO: 41 (chain 1) and a light chain comprising the amino acid sequence of SEQ ID NO: 50 (chain 2), and a heavy chain comprising the amino acid sequence of SEQ ID NO: 54 (chain 3) and a light chain comprising the amino acid sequence of SEQ ID NO: 68 (chain 4);
(h02) a heavy chain comprising the amino acid sequence of SEQ ID NO: 41 (chain 1) and a light chain comprising the amino acid sequence of SEQ ID NO: 50 (chain 2), and a heavy chain comprising the amino acid sequence of SEQ ID NO: 55 (chain 3) and a light chain comprising the amino acid sequence of SEQ ID NO: 68 (chain 4);
(h03) a heavy chain comprising the amino acid sequence of SEQ ID NO: 42 (chain 1) and a light chain comprising the amino acid sequence of SEQ ID NO: 51 (chain 2), and a heavy chain comprising the amino acid sequence of SEQ ID NO: 56 (chain 3) and a light chain comprising the amino acid sequence of SEQ ID NO: 69 (chain 4);
(h04) a heavy chain comprising the amino acid sequence of SEQ ID NO: 42 (chain 1) and a light chain comprising the amino acid sequence of SEQ ID NO: 51 (chain 2), and a heavy chain comprising the amino acid sequence of SEQ ID NO: 57 (chain 3) and a light chain comprising the amino acid sequence of SEQ ID NO: 69 (chain 4);
(h05) a heavy chain comprising the amino acid sequence of SEQ ID NO: 44 (chain 1) and a light chain comprising the amino acid sequence of SEQ ID NO: 52 (chain 2), and a heavy chain comprising the amino acid sequence of SEQ ID NO: 60 (chain 3) and a light chain comprising the amino acid sequence of SEQ ID NO: 70 (chain 4);
(h06) a heavy chain comprising the amino acid sequence of SEQ ID NO: 44 (chain 1) and a light chain comprising the amino acid sequence of SEQ ID NO: 52 (chain 2), and a heavy chain comprising the amino acid sequence of SEQ ID NO: 61 (chain 3) and a light chain comprising the amino acid sequence of SEQ ID NO: 70 (chain 4);
(h07) a heavy chain comprising the amino acid sequence of SEQ ID NO: 45 (chain 1) and a light chain comprising the amino acid sequence of SEQ ID NO: 50 (chain 2), and a heavy chain comprising the amino acid sequence of SEQ ID NO: 62 (chain 3) and a light chain comprising the amino acid sequence of SEQ ID NO: 68 (chain 4);
(h08) a heavy chain comprising the amino acid sequence of SEQ ID NO: 45 (chain 1) and a light chain comprising the amino acid sequence of SEQ ID NO: 50 (chain 2), and a heavy chain comprising the amino acid sequence of SEQ ID NO: 63 (chain 3) and a light chain comprising the amino acid sequence of SEQ ID NO: 68 (chain 4);
(h09) a heavy chain comprising the amino acid sequence of SEQ ID NO: 46 (chain 1) and a light chain comprising the amino acid sequence of SEQ ID NO: 51 (chain 2), and a heavy chain comprising the amino acid sequence of SEQ ID NO: 64 (chain 3) and a light chain comprising the amino acid sequence of SEQ ID NO: 69 (chain 4);
(h10) a heavy chain comprising the amino acid sequence of SEQ ID NO: 46 (chain 1) and a light chain comprising the amino acid sequence of SEQ ID NO: 51 (chain 2), and a heavy chain comprising the amino acid sequence of SEQ ID NO: 65 (chain 3) and a light chain comprising the amino acid sequence of SEQ ID NO: 69 (chain 4);
(h11) a heavy chain comprising the amino acid sequence of SEQ ID NO: 47 (chain 1) and a light chain comprising the amino acid sequence of SEQ ID NO: 52 (chain 2), and a heavy chain comprising the amino acid sequence of SEQ ID NO: 66 (chain 3) and a light chain comprising the amino acid sequence of SEQ ID NO: 70 (chain 4);
(h12) a heavy chain comprising the amino acid sequence of SEQ ID NO: 47 (chain 1) and a light chain comprising the amino acid sequence of SEQ ID NO: 52 (chain 2), and a heavy chain comprising the amino acid sequence of SEQ ID NO: 67 (chain 3) and a light chain comprising the amino acid sequence of SEQ ID NO: 70 (chain 4);
(h13) a heavy chain comprising the amino acid sequence of SEQ ID NO: 48 (chain 1) and a light chain comprising the amino acid sequence of SEQ ID NO: 53 (chain 2), and a heavy chain comprising the amino acid sequence of SEQ ID NO: 56 (chain 3) and a light chain comprising the amino acid sequence of SEQ ID NO: 71 (chain 4);
(h14) a heavy chain comprising the amino acid sequence of SEQ ID NO: 48 (chain 1) and a light chain comprising the amino acid sequence of SEQ ID NO: 53 (chain 2), and a heavy chain comprising the amino acid sequence of SEQ ID NO: 57 (chain 3) and a light chain comprising the amino acid sequence of SEQ ID NO: 71 (chain 4);
(h15) a heavy chain comprising the amino acid sequence of SEQ ID NO: 49 (chain 1) and a light chain comprising the amino acid sequence of SEQ ID NO: 53 (chain 2), and a heavy chain comprising the amino acid sequence of SEQ ID NO: 64 (chain 3) and a light chain comprising the amino acid sequence of SEQ ID NO: 71 (chain 4);
(h16) a heavy chain comprising the amino acid sequence of SEQ ID NO: 49 (chain 1) and a light chain comprising the amino acid sequence of SEQ ID NO: 53 (chain 2), and a heavy chain comprising the amino acid sequence of SEQ ID NO: 65 (chain 3) and a light chain comprising the amino acid sequence of SEQ ID NO: 71 (chain 4);
(h17) a heavy chain comprising the amino acid sequence of SEQ ID NO: 43 (chain 1) and a light chain comprising the amino acid sequence of SEQ ID NO: 52 (chain 2), and a heavy chain comprising the amino acid sequence of SEQ ID NO: 58 (chain 3) and a light chain comprising the amino acid sequence of SEQ ID NO: 70 (chain 4);
(h18) a heavy chain comprising the amino acid sequence of SEQ ID NO: 43 (chain 1) and a light chain comprising the amino acid sequence of SEQ ID NO: 52 (chain 2), and a heavy chain comprising the amino acid sequence of SEQ ID NO: 59 (chain 3) and a light chain comprising the amino acid sequence of SEQ ID NO: 70 (chain 4).

### Pyroglutamylation

It is known that when an antibody is expressed in cells, the antibody is modified after translation. Examples of the posttranslational modification include cleavage of lysine at the C terminal of the heavy chain by a carboxypeptidase; modification of glutamine or glutamic acid at the N terminal of the heavy chain and the light chain to pyroglutamic acid by pyroglutamylation; glycosylation; oxidation; deamidation; and glycation, and it is known that such posttranslational modifications occur in various antibodies (Journal of Pharmaceutical Sciences, 2008, Vol. 97, p. 2426-2447).

The multispecific antigen-binding molecules of the present disclosure also includes a multispecific antibody which has undergone posttranslational modification. Examples of the multispecific antigen-binding molecules thereof of the present disclosure, which undergoes posttranslational modification, include multispecific antibodies which have undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

### Antigen Binding Moiety

As used herein, the term “antigen binding moiety” refers to a polypeptide molecule that specifically binds to an antigen. In one embodiment, an antigen binding moiety is able to direct the entity to which it is attached (e.g. a second antigen binding moiety) to a target site, for example to a specific type of tumor cell expressing the cancer antigen (CLDN6). In another embodiment an antigen binding moiety is able to activate signaling through its target antigen, for example a T cell receptor complex antigen (CD3) or co-stimulatory molecule CD137. Antigen binding moieties include antibodies and fragments thereof as further defined herein. Particular antigen binding moieties include an antigen binding domain or an antibody variable region of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: alpha, delta, epsilon, gamma, or mu. Useful light chain constant regions include any of the two isotypes: kappa and lambda.

As used herein, the terms “first”, “second”, “third”, and “fourth” with respect to antigen binding moieties etc., are used for convenience of distinguishing when there is more than one of each type of moiety and such. Use of these terms is not intended to confer a specific order or orientation of the multispecific antigen-binding molecule unless explicitly so stated.

### Antigen-Binding Moiety Capable of Binding to CD3 and CD137 but not at the Same Time

The multispecific antigen-binding molecule described herein comprises at least one antigen binding moiety capable of binding to CD3 and CD137, but does not bind to CD3 and CD137 at the same time (also referred to herein as “Dual antigen-binding moiety” or “first antigen-binding moiety” or “Dual-Ig” or “Dual-Fab”). In a particular embodiment, the multispecific antigen-binding molecule comprises not more than two antigen binding moiety capable of specific binding to CD3 and CD137 but does not bind to CD3 and CD137 at the same time. In one embodiment, the multispecific antigen-binding molecule provides monovalent binding to CD3 or CD137, but does not bind to CD3 and CD137 at the same time.

In certain embodiments, the Dual antigen-binding moiety (“first antigen-binding moiety”) is generally a Fab molecule, particularly a conventional Fab molecule. In certain embodiments, the Dual antigen binding moiety (“first antigen-binding moiety”) is a domain comprising antibody light-chain and heavy-chain variable regions (VL and VH). Suitable examples of such domains comprising antibody light-chain and heavy-chain variable regions include “single chain Fv (scFv)”, “single chain antibody”, “Fv”, “single chain Fv 2 (scFv2)”, “Fab”, “F(ab′)2”, etc.

In certain embodiments, the Dual antigen-binding moiety (“first antigen-binding moiety”) specifically binds to the whole or a portion of a partial peptide of CD3. In a particular embodiment, CD3 is human CD3 or cynomolgus CD3, most particularly human CD3. In a particular embodiment, the first antigen-binding moiety is cross-reactive for (i.e. specifically binds to) human and cynomolgus CD3. In some embodiments, the first antigen-binding moiety is capable of specific binding to the epsilon subunit of CD3, in particular the human CD3 epsilon subunit of CD3 which is shown in SEQ ID NO: 170 (NP_000724.1) (RefSeq registration numbers are shown within the parentheses). In some embodiments, the first antigen-binding moiety is capable of specific binding to the CD3 epsilon chain expressed on the surface of eukaryotic cells. In some embodiments, the first antigen-binding moiety binds to the CD3 epsilon chain expressed on the surface of T cells.

In certain embodiments, the CD137 is human CD137. In some embodiments, favorable examples of an antigen-binding molecule of the present disclosure include antigen-binding molecules that bind to the same epitope as the human CD137 epitope bound by the antibody selected from the group consisting of: antibody that recognize a region comprising the SPCPPNSFSSAGGQRTCDICRQC KGVFRTRKECSSTSNAECDCTPGFHCLGAGCSMCEQDCKQGQELTKKGC sequence (SEQ ID NO: 182), antibody that recognize a region comprising the DCTPGFHCLGAGCSMCEQDCKQGQ ELTKKGC sequence (SEQ ID NO: 181), antibody that recognize a region comprising the LQDPCSNCPAGTFCDNNRNQICSP CPPNSFSSAGGQRTCDICRQCKGVFRTRKECSSTSNAEC sequence (SEQ ID NO: 183), and antibody that recognize a region comprising the LQDPCSNCPAGTFCDNNRNQIC sequence (SEQ ID NO: 180) in the human CD137 protein.

In specific embodiments, the Dual antigen-binding moiety (“first antigen-binding moiety”) comprises any one of the antibody variable regions of (a1) to (a4) below:
(a1) a heavy chain variable region comprising the complementarity determining region (CDR) 1 of SEQ ID NO: 9, the CDR 2 of SEQ ID NO: 15, and the CDR 3 of SEQ ID NO: 21, and a light chain variable region comprising the CDR 1 of SEQ ID NO: 31, the CDR 2 of SEQ ID NO: 35, and the CDR 3 of SEQ ID NO: 39;
(a2) a heavy chain variable region comprising the complementarity determining region (CDR) 1 of SEQ ID NO: 10, the CDR 2 of SEQ ID NO: 16, and the CDR 3 of SEQ ID NO: 22, and a light chain variable region comprising the CDR 1 of SEQ ID NO: 31, the CDR 2 of SEQ ID NO: 35, and the CDR 3 of SEQ ID NO: 39;
(a3) a heavy chain variable region comprising the complementarity determining region (CDR) 1 of SEQ ID NO: 11, the CDR 2 of SEQ ID NO: 17, and the CDR 3 of SEQ ID NO: 23, and a light chain variable region comprising the CDR 1 of SEQ ID NO: 32, the CDR 2 of SEQ ID NO: 36, and the CDR 3 of SEQ ID NO: 40;
(a4) a heavy chain variable region comprising the complementarity determining region (CDR) 1 of SEQ ID NO: 12, the CDR 2 of SEQ ID NO: 18, and the CDR 3 of SEQ ID NO: 24, and a light chain variable region comprising the CDR 1 of SEQ ID NO: 32, the CDR 2 of SEQ ID NO: 36, and the CDR 3 of SEQ ID NO: 40.

In specific embodiments, the Dual antigen-binding moiety (“first antigen-binding moiety”) comprises the antibody variable regions that comprise human antibody frameworks or humanized antibody frameworks.

In specific embodiments, the Dual antigen-binding moiety (“first antigen-binding moiety”) comprises any one of (c1) to (c4) below:
(c1) a heavy chain variable region comprising the amino acid sequence of SEQ ID NO: 3, and a light chain variable region comprising the amino acid sequence of SEQ ID NO: 27;
(c2) a heavy chain variable region comprising the amino acid sequence of SEQ ID NO: 4, and a light chain variable region comprising the amino acid sequence of SEQ ID NO: 27;
(c3) a heavy chain variable region comprising the amino acid sequence of SEQ ID NO: 5, and a light chain variable region comprising the amino acid sequence of SEQ ID NO: 28;
(c4) a heavy chain variable region comprising the amino acid sequence of SEQ ID NO: 6, and a s light chain variable region comprising the amino acid sequence of SEQ ID NO: 28.

In one embodiment, the Dual antigen-binding moiety (“first antigen-binding moiety”) comprises a heavy chain variable region sequence that is at least about 95%, 96%, 97%, 98%, 99% or 100% identical to SEQ ID NO: 3 and a light chain variable region sequence that is at least about 95%, 96%, 97%, 98%, 99% or 100% identical to SEQ ID NO: 27.

In one embodiment, the Dual antigen-binding moiety (“first antigen-binding moiety”) comprises a heavy chain variable region sequence that is at least about 95%, 96%, 97%, 98%, 99% or 100% identical to SEQ ID NO: 4 and a light chain variable region sequence that is at least about 95%, 96%, 97%, 98%, 99% or 100% identical to SEQ ID NO: 27.

In one embodiment, the Dual antigen-binding moiety (“first antigen-binding moiety”) comprises a heavy chain variable region sequence that is at least about 95%, 96%, 97%, 98%, 99% or 100% identical to SEQ ID NO: 5 and a light chain variable region sequence that is at least about 95%, 96%, 97%, 98%, 99% or 100% identical to SEQ ID NO: 28.

In one embodiment the Dual antigen-binding moiety (“first antigen-binding moiety”) comprises a heavy chain variable region sequence that is at least about 95%, 96%, 97%, 98%, 99% or 100% identical to SEQ ID NO: 6 and a light chain variable region sequence that is at least about 95%, 96%, 97%, 98%, 99% or 100% identical to SEQ ID NO: 28.

In specific embodiments, the Dual antigen-binding moiety (“first antigen-binding moiety”) comprises any one of (j01) to (j18) below:
(j01) a heavy chain comprising the amino acid sequence of SEQ ID NO: 54 (chain 3) and a light chain comprising the amino acid sequence of SEQ ID NO: 68 (chain 4);
(j02) a heavy chain comprising the amino acid sequence of SEQ ID NO: 55 (chain 3) and a light chain comprising the amino acid sequence of SEQ ID NO: 68 (chain 4);
(j03) a heavy chain comprising the amino acid sequence of SEQ ID NO: 56 (chain 3) and a light chain comprising the amino acid sequence of SEQ ID NO: 69 (chain 4);
(j04) a heavy chain comprising the amino acid sequence of SEQ ID NO: 57 (chain 3) and a light chain comprising the amino acid sequence of SEQ ID NO: 69 (chain 4);
(j05) a heavy chain comprising the amino acid sequence of SEQ ID NO: 60 (chain 3) and a light chain comprising the amino acid sequence of SEQ ID NO: 70 (chain 4);
(j06) a heavy chain comprising the amino acid sequence of SEQ ID NO: 61 (chain 3) and a light chain comprising the amino acid sequence of SEQ ID NO: 70 (chain 4);
(j07) a heavy chain comprising the amino acid sequence of SEQ ID NO: 62 (chain 3) and a light chain comprising the amino acid sequence of SEQ ID NO: 68 (chain 4);
(j08) a heavy chain comprising the amino acid sequence of SEQ ID NO: 63 (chain 3) and a light chain comprising the amino acid sequence of SEQ ID NO: 68 (chain 4);
(j09) a heavy chain comprising the amino acid sequence of SEQ ID NO: 64 (chain 3) and a light chain comprising the amino acid sequence of SEQ ID NO: 69 (chain 4);

(j10) a heavy chain comprising the amino acid sequence of SEQ ID NO: 65 (chain 3) and a light chain comprising the amino acid sequence of SEQ ID NO: 69 (chain 4);
(j11) a heavy chain comprising the amino acid sequence of SEQ ID NO: 66 (chain 3) and a light chain comprising the amino acid sequence of SEQ ID NO: 70 (chain 4);
(j12) a heavy chain comprising the amino acid sequence of SEQ ID NO: 67 (chain 3) and a light chain comprising the amino acid sequence of SEQ ID NO: 70 (chain 4);
(j13) a heavy chain comprising the amino acid sequence of SEQ ID NO: 56 (chain 3) and a light chain comprising the amino acid sequence of SEQ ID NO: 71 (chain 4);
(j14) a heavy chain comprising the amino acid sequence of SEQ ID NO: 57 (chain 3) and a light chain comprising the amino acid sequence of SEQ ID NO: 71 (chain 4);
(j15) a heavy chain comprising the amino acid sequence of SEQ ID NO: 64 (chain 3) and a light chain comprising the amino acid sequence of SEQ ID NO: 71 (chain 4);
(j16) a heavy chain comprising the amino acid sequence of SEQ ID NO: 65 (chain 3) and a light chain comprising the amino acid sequence of SEQ ID NO: 71 (chain 4);
(j17) a heavy chain comprising the amino acid sequence of SEQ ID NO: 58 (chain 3) and a light chain comprising the amino acid sequence of SEQ ID NO: 70 (chain 4);
(j18) a heavy chain comprising the amino acid sequence of SEQ ID NO: 59 (chain 3) and a light chain comprising the amino acid sequence of SEQ ID NO: 70 (chain 4).

The multispecific antigen-binding molecules of the present disclosure also include a multispecific antibody which has undergone posttranslational modification. Examples of the multispecific antigen-binding molecules thereof of the present disclosure, which undergoes posttranslational modification, include multispecific antigen-binding molecules which have undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

### Antigen-Binding Moiety Capable of Binding to CLDN6

The multispecific antigen-binding molecule described herein comprises at least one antigen-binding moiety capable of binding to CLDN6 (also referred to herein as a “CLDN6 antigen-binding moiety” or “second antigen-binding moiety”). In certain embodiments, the multispecific antigen-binding molecule comprises one antigen-binding moiety capable of binding to CLDN6.

In certain embodiments, the CLDN6 antigen-binding moiety (“second antigen-binding moiety”) is generally a Fab molecule, particularly a conventional Fab molecule. In certain embodiments, the CLDN6 antigen-binding moiety (“second antigen-binding moiety”) is a domain comprising antibody light-chain and heavy-chain variable regions (VL and VH). Suitable examples of such domains comprising antibody light-chain and heavy-chain variable regions include “single chain Fv (scFv)”, “single chain antibody”, “Fv”, “single chain Fv 2 (scFv2)”, “Fab”, “F(ab′)2”, etc.

In certain embodiments, the CLDN6 antigen-binding moiety (“second antigen-binding moiety”) specifically binds to the whole or a portion of a partial peptide of CLDN6. In a particular embodiment CLDN6 is human CLDN6 or cynomolgus CLDN6 or mouse CLDN6, most particularly human CLDN6. In a particular embodiment, the CLDN6 antigen-binding moiety (“second antigen-binding moiety”) is cross-reactive for (i.e. specifically binds to) human and cynomolgus CLDN6.

In certain embodiments, the CLDN6 antigen-binding moiety (“second antigen-binding moiety”) specifically binds to the first extracellular domain of CLDN6 (amino acids 29-81 of SEQ ID NO: 196 or 197) or the second extracellular domain of CLDN6 (amino acids 138-159 of SEQ ID NO: 196 or 197). In certain embodiments, the CLDN6 antigen-binding moiety (“second antigen-binding moiety”) specifically binds to human CLDN6 expressed on the surface of eukaryotic cells. In certain embodiments, binding activity towards CLDN6 is the binding activity towards the CLDN6 protein expressed on the surface of cancer cells.

In certain embodiments, the CLDN6 antigen-binding moiety (“second antigen-binding moiety”) does not substantially bind to human CLDN9.

In certain embodiments, the CLDN6 antigen-binding moiety (“second antigen-binding moiety”) does not substantially bind to human CLDN4.

In certain embodiments, the CLDN6 antigen-binding moiety (“second antigen-binding moiety”) does not substantially bind to human CLDN3.

In certain embodiments, the CLDN6 antigen-binding moiety (“second antigen-binding moiety”) does not substantially bind to a CLDN6 mutant as defined in SEQ ID NO:205.

In certain embodiments, the CLDN6 antigen-binding moiety (“second antigen-binding moiety”) is a crossover Fab molecule, i.e. a Fab molecule wherein either the variable or the constant regions of the Fab heavy and light chains are exchanged.

In specific embodiments, the CLDN6 antigen-binding moiety (“second antigen-binding moiety”) comprises the antibody variable regions of (b1) or (b2) below:
(b1) a heavy chain variable region comprising the complementarity determining region (CDR) 1 of SEQ ID NO: 8, the CDR 2 of SEQ ID NO: 14, and the CDR 3 of SEQ ID NO: 20, and a light chain variable region comprising the CDR 1 of SEQ ID NO: 30, the CDR 2 of SEQ ID NO: 34, and the CDR 3 of SEQ ID NO: 38;
(b2) a heavy chain variable region comprising the complementarity determining region (CDR) 1 of SEQ ID NO: 7, the CDR 2 of SEQ ID NO: 13, and the CDR 3 of SEQ ID NO: 19, and a light chain variable region comprising the CDR 1 of SEQ ID NO: 29, the CDR 2 of SEQ ID NO: 33, and the CDR 3 of SEQ ID NO: 37.

In specific embodiments, the CLDN6 antigen-binding moiety (“second antigen-binding moiety”) comprises the antibody variable regions that comprise human antibody frameworks or humanized antibody frameworks.

In specific embodiments, the CLDN6 antigen-binding moiety (“second antigen-binding moiety”) comprises (d1) or (d2) below:
(d1) a heavy chain variable region comprising the amino acid sequence of SEQ ID NO: 2, and a light chain variable region comprising the amino acid sequence of SEQ ID NO: 26;
(d2) a heavy chain variable region comprising the amino acid sequence of SEQ ID NO: 1, and a light chain variable region comprising the amino acid sequence of SEQ ID NO: 25.

In one embodiment, the CLDN6 antigen-binding moiety (“second antigen-binding moiety”) comprises a heavy chain variable region sequence that is at least about 95%, 96%, 97%, 98%, 99% or 100% identical to SEQ ID NO: 2 and a light chain variable region sequence that is at least about 95%, 96%, 97%, 98%, 99% or 100% identical to SEQ ID NO: 26.

In one embodiment, the CLDN6 antigen-binding moiety (“second antigen-binding moiety”) comprises a heavy chain variable region sequence that is at least about 95%, 96%, 97%, 98%, 99% or 100% identical to SEQ ID NO: 1 and a light chain variable region sequence that is at least about 95%, 96%, 97%, 98%, 99% or 100% identical to SEQ ID NO: 25.

In specific embodiments, the CLDN6 antigen-binding moiety (“second antigen-binding moiety”) comprises any one of (k01) to (k09) below:
(k01) a heavy chain comprising the amino acid sequence of SEQ ID NO: 41 (chain 1) and a light chain comprising the amino acid sequence of SEQ ID NO: 50 (chain 2);
(k02) a heavy chain comprising the amino acid sequence of SEQ ID NO: 42 (chain 1) and a light chain comprising the amino acid sequence of SEQ ID NO: 51 (chain 2);
(k03) a heavy chain comprising the amino acid sequence of SEQ ID NO: 44 (chain 1) and a light chain comprising the amino acid sequence of SEQ ID NO: 52 (chain 2);
(k04) a heavy chain comprising the amino acid sequence of SEQ ID NO: 45 (chain 1) and a light chain comprising the amino acid sequence of SEQ ID NO: 50 (chain 2);
(k05) a heavy chain comprising the amino acid sequence of SEQ ID NO: 46 (chain 1) and a light chain comprising the amino acid sequence of SEQ ID NO: 51 (chain 2);
(k06) a heavy chain comprising the amino acid sequence of SEQ ID NO: 47 (chain 1) and a light chain comprising the amino acid sequence of SEQ ID NO: 52 (chain 2);
(k07) a heavy chain comprising the amino acid sequence of SEQ ID NO: 48 (chain 1) and a light chain comprising the amino acid sequence of SEQ ID NO: 53 (chain 2);
(k08) a heavy chain comprising the amino acid sequence of SEQ ID NO: 49 (chain 1) and a light chain comprising the amino acid sequence of SEQ ID NO: 53 (chain 2);
(k09) a heavy chain comprising the amino acid sequence of SEQ ID NO: 43 (chain 1) and a light chain comprising the amino acid sequence of SEQ ID NO: 52 (chain 2).

The multispecific antigen-binding molecules of the present disclosure also include a multispecific antibody which has undergone posttranslational modification. Examples of the multispecific antigen-binding molecules thereof of the present disclosure, which undergoes posttranslational modification, include multispecific antibodies which have undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

### Antigen

As used herein, the term “antigen” refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g. CD3, CD137, CLDN6) can be any native form the proteins from any vertebrate source, including mammals such as primates (e.g. humans) and rodents (e.g. mice and rats), unless otherwise indicated. In a particular embodiment the antigen is a human CD3, human CD137 or human CLDN6. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

In certain embodiments, the multispecific antigen-binding molecule described herein binds to an epitope of CD3, CD137 or CLDN6 that is conserved among the CD3, CD137 or CLDN6 from different species. In certain embodiments, the multispecific antigen-binding molecule of the present application is a trispecific antigen-binding molecule, i.e. it is capable of specifically binding to three different antigens—capable of binding to either one of CD3 or CD137 but does not bind to both antigens simultaneously, and is capable of specifically binding to CLDN6.

### Claudin-6 (CLDN6) and Other Claudin Family Proteins

The term “CLDN6”, as used herein, refers to any native Claudin-6 from any vertebrate source, including mammals such as primates (e.g. humans) and rodents (e.g., mice and rats), unless otherwise indicated. The amino acid sequence of human CLDN6 (hCLDN6) is shown in SEQ ID NO: 196 or 197, and the amino acid sequence of mouse CLDN6 (mCLDN6) is shown in SEQ ID NO: 201.

There are many other proteins within Claudin family other than CLDN6, such as CLDN3, CLDN4, and CLDN9. The amino acid sequences of human CLDN3 (hCLDN3), human CLDN4 (hCLDN4) and human CLDN9 (hCLDN9) are shown in SEQ ID NOs:199, 200 and 198, respectively. The amino acid sequences of mouse CLDN3 (mCLDN3), mouse CLDN4 (mCLDN4) and mouse CLDN9 (mCLDN9) are shown in SEQ ID NOs: 203, 204 and 202, respectively.

### CD3

In certain embodiments, the multispecific antigen-binding molecule specifically binds to the whole or a portion of a partial peptide of CD3. In a particular embodiment, CD3 is human CD3 or cynomolgus CD3, most particularly human CD3. In a particular embodiment the multispecific antigen-binding molecule is cross-reactive for (i.e. specifically binds to) human and cynomolgus CD3. In some embodiments, the multispecific antigen-binding molecule is capable of specific binding to the epsilon subunit of CD3, in particular the human CD3 epsilon subunit of CD3 which is shown in SEQ ID NO: 170 (NP_000724.1) (RefSeq registration numbers are shown within the parentheses). In some embodiments, the multispecific antigen-binding molecule is capable of specific binding to the CD3 epsilon chain expressed on the surface of eukaryotic cells. In some embodiments, the multispecific antigen-binding molecule binds to the CD3 epsilon chain expressed on the surface of T cells.

### CD137

In certain embodiments, the CD137 is human CD137. In some embodiments, favorable examples of an antigen-binding molecule of the present disclosure include antigen-binding molecules that bind to the same epitope as the human CD137 epitope bound by the antibody selected from the group consisting of: antibody that recognize a region comprising the SPCPPNSFSSAGGQRTCDICRQC KGVFR TRKECSSTSNAECDCTPGFHCLGAGCSMCEQDCKQGQELTKKGC sequence (SEQ ID NO: 182), antibody that recognize a region comprising the DCTPGFHCLGAGCSMCEQDC KQGQELTKKGC sequence (SEQ ID NO: 181), antibody that recognize a region comprising the LQDPCSNCPAGTFCDNNRN QICSPCPPNSFSSAGGQRTCDICRQCKGVFRTRKECSSTSNAEC sequence (SEQ ID NO: 183), and antibody that recognize a region comprising the LQDPCSNCPAGTFCD NNRN QIC sequence (SEQ ID NO: 180) in the human CD137 protein.

### Antigen-Binding Domain

The term “antigen-binding domain” refers to the part of an antibody that comprises the area which specifically binds to and is complementary to part or all of an antigen. An antigen-binding domain may be provided by, for example, one or more antibody variable domains (also called antibody variable regions). Preferably, the antigen-binding domains contain both the antibody light chain variable region (VL) and antibody heavy chain variable region (VH). Such preferable antigen-binding domains include, for example, “single-chain Fv (scFv)”, “single-chain antibody”, “Fv”, “single-chain Fv2 (scFv2)”, “Fab”, and “F (ab′)2”. An antigen-binding domain may also be provided by single-domain antibodies.

### Single-Domain Antibody

In the present specification, the term “single-domain antibody” is not limited by its structure as long as the domain can exert antigen binding activity by itself. It is known that a general antibody, for example, an IgG antibody, exhibits antigen binding activity in a state where a variable region is formed by the pairing of VH and VL, whereas the own domain structure of the single-domain antibody can exert antigen binding activity by itself without pairing with another domain. Usually, the single-domain antibody has a relatively low molecular weight and exists in the form of a monomer.

Examples of the single-domain antibody include, but are not limited to, antigen-binding molecules congenitally lacking a light chain, such as VHH of an animal of the family Camelidae and shark VNAR, and antibody fragments containing the whole or a portion of an antibody VH domain or the whole or a portion of an antibody VL domain. Examples of the single-domain antibody which is an antibody fragment containing the whole or a portion of an antibody VH or VL domain include, but are not limited to, artificially prepared single-domain antibodies originating from human antibody VH or human antibody VL as described in U.S. Pat. No. 6,248,516 B1, etc. In some embodiments of the present invention, one single-domain antibody has three CDRs (CDR1, CDR2 and CDR3).

The single-domain antibody can be obtained from an animal capable of producing the single-domain antibody or by the immunization of the animal capable of producing the single-domain antibody. Examples of the animal capable of producing the single-domain antibody include, but are not limited to, animals of the family Camelidae, and transgenic animals harboring a gene capable of raising the single-domain antibody. The animals of the family Camelidae include camels, lamas, alpacas, one-hump camels and guanacos, etc. Examples of the transgenic animals harboring a gene capable of raising the single-domain antibody include, but are not limited to, transgenic animals described in International Publication No. WO2015/143414 and U.S. Patent Publication No. US2011/0123527 A1. The framework sequences of the single-domain antibody obtained from the animal may be converted to human germline sequences or sequences similar thereto to obtain a humanized single-domain antibody. The humanized single-domain antibody (e.g., humanized VHH) is also one embodiment of the single-domain antibody of the present invention.

Alternatively, the single-domain antibody can be obtained by ELISA, panning, or the like from a polypeptide library containing single-domain antibodies. Examples of the polypeptide library containing single-domain antibodies include, but are not limited to, naive antibody libraries obtained from various animals or humans (e.g., Methods in Molecular Biology 2012 911 (65-78); and Biochimica et Biophysica Acta—Proteins and Proteomics 2006 1764: 8 (1307-1319)), antibody libraries obtained by the immunization of various animals (e.g., Journal of Applied Microbiology 2014 117: 2 (528-536)), and synthetic antibody libraries prepared from antibody genes of various animals or humans (e.g., Journal of Biomolecular Screening 2016 21: 1 (35-43); Journal of Biological Chemistry 2016 291:24 (12641-12657); and AIDS 2016 30: 11 (1691-1701)).

### Variable Region

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### HVR or CDR

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Hypervariable regions (HVRs) are also referred to as “complementarity determining regions” (CDRs), and these terms are used herein interchangeably in reference to portions of the variable region that form the antigen binding regions. Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

HVR-H1, HVR-H2, HVR-H3, HVR-L1, HVR-L2, and HVR-L3 are also mentioned as “H-CDR1”, “H-CDR2”, “H-CDR3”, “L-CDR1”, “L-CDR2”, and “L-CDR3”, respectively.

### Capable of Binding to CD3 and CD137

Whether the antibody variable region of the present disclosure is “capable of binding to CD3 and CD137” can be determined by a method known in the art.

This can be determined by, for example, an electrochemiluminescence method (ECL method) (BMC Research Notes 2011, 4: 281).

Specifically, for example, a low-molecular antibody composed of a region capable of binding to CD3 and CD137, for example, a Fab region, of a biotin-labeled antigen-binding molecule to be tested, or a monovalent antibody (antibody lacking one of the two Fab regions carried by a usual antibody) thereof is mixed with CD3 or CD137 labeled with sulfo-tag (Ru complex), and the mixture is added onto a streptavidin-immobilized plate. In this operation, the biotin-labeled antigen-binding molecule to be tested binds to streptavidin on the plate. Light is developed from the sulfo-tag, and the luminescence signal can be detected using Sector Imager 600 or 2400 (MSD K.K.) or the like to thereby confirm the binding of the aforementioned region of the antigen-binding molecule to be tested to CD3 or CD137.

Alternatively, this assay may be conducted by ELISA, FACS (fluorescence activated cell sorting), ALPHAScreen (amplified luminescent proximity homogeneous assay screen), the BIACORE method based on a surface plasmon resonance (SPR) phenomenon, etc. (Proc. Natl. Acad. Sci. USA (2006) 103 (11), 4005-4010).

Specifically, the assay can be conducted using, for example, an interaction analyzer Biacore™ (GE Healthcare Japan Corp.) based on a surface plasmon resonance (SPR) phenomenon. The Biacore™ analyzer includes any model such as Biacore T100, T200, X100, A100, 4000, 3000, 2000, 1000, or C. Any sensor chip for Biacore, such as a CM7, CM5, CM4, CM3, C1, SA, NTA, L1, HPA, or Au chip, can be used as a sensor chip. Proteins for capturing the antigen-binding molecule of the present disclosure, such as protein A, protein G, protein L, anti-human IgG antibodies, anti-human IgG-Fab, anti-human L chain antibodies, anti-human Fc antibodies, antigenic proteins, or antigenic peptides, are immobilized onto the sensor chip by a coupling method such as amine coupling, disulfide coupling, or aldehyde coupling. CD3 or CD137 is injected thereon as an analyte, and the interaction is measured to obtain a sensorgram. In this operation, the concentration of CD3 or CD137 can be selected within the range of a few micro M to a few pM according to the interaction strength (e.g., KD) of the assay sample.

Alternatively, CD3 or CD137 may be immobilized instead of the antigen-binding molecule onto the sensor chip, with which the antibody sample to be evaluated is in turn allowed to interact. Whether the antibody variable region of the antigen-binding molecule of the present disclosure has binding activity against CD3 or CD137 can be confirmed on the basis of a dissociation constant (KD) value calculated from the sensorgram of the interaction or on the basis of the degree of increase in the sensorgram after the action of the antigen-binding molecule sample over the level before the action.

In some embodiments, binding activity or affinity of the antibody variable region of the present disclosure to the antigen of interest (i.e. CD3 or CD137) are assessed at 37 degrees C. (for CD137) or 25 degrees C. (for CD3) using e.g., Biacore T200 instrument (GE Healthcare) or Biacore 8K instrument (GE Healthcare). Anti-human Fc (e.g., GE Healthcare) is immobilized onto all flow cells of a CM4 sensor chip using amine coupling kit (e.g, GE Healthcare). The antigen binding molecules or antibody variable regions are captured onto the anti-Fc sensor surfaces, then the antigen (CD3 or CD137) is injected over the flow cell. The capture levels of the antigen binding molecules or antibody variable regions may be aimed at 200 resonance unit (RU). Recombinant human CD3 or CD137 may be injected at 400 to 25 nM prepared by two-fold serial dilution, followed by dissociation. All antigen binding molecules or antibody variable regions and analytes are prepared in ACES pH 7.4 containing 20 mM ACES, 150 mM NaCl, 0.05% Tween 20, 0.005% NaN3. Sensor surface is regenerated each cycle with 3M MgCl2. Binding affinity are determined by processing and fitting the data to 1:1 binding model using e.g., Biacore T200 Evaluation software, version 2.0 (GE Healthcare) or Biacore 8K Evaluation software (GE Healthcare). The KD values are calculated for assessing the specific binding activity or affinity of the antigen binding domains of the present disclosure.

The ALPHAScreen is carried out by the ALPHA technology using two types of beads (donor and acceptor) on the basis of the following principle: luminescence signals are detected only when these two beads are located in proximity through the biological interaction between a molecule bound with the donor bead and a molecule bound with the acceptor bead. A laser-excited photosensitizer in the donor bead converts ambient oxygen to singlet oxygen having an excited state. The singlet oxygen diffuses around the donor bead and reaches the acceptor bead located in proximity thereto to thereby cause chemiluminescent reaction in the bead, which finally emits light. In the absence of the interaction between the molecule bound with the donor bead and the molecule bound with the acceptor bead, singlet oxygen produced by the donor bead does not reach the acceptor bead. Thus, no chemiluminescent reaction occurs.

One (ligand) of the substances between which the interaction is to be observed is immobilized onto a thin gold film of a sensor chip. The sensor chip is irradiated with light from the back such that total reflection occurs at the interface between the thin gold film and glass. As a result, a site having a drop in reflection intensity (SPR signal) is formed in a portion of reflected light. The other (analyte) of the substances between which the interaction is to be observed is injected on the surface of the sensor chip. Upon binding of the analyte to the ligand, the mass of the immobilized ligand molecule is increased to change the refractive index of the solvent on the sensor chip surface. This change in the refractive index shifts the position of the SPR signal (on the contrary, the dissociation of the bound molecules gets the signal back to the original position). The Biacore system plots on the ordinate the amount of the shift, i.e., change in mass on the sensor chip surface, and displays time-dependent change in mass as assay data (sensorgram). The amount of the analyte bound to the ligand captured on the sensor chip surface (amount of change in response on the sensorgram between before and after the interaction of the analyte) can be determined from the sensorgram. However, since the amount bound also depends on the amount of the ligand, the comparison must be performed under conditions where substantially the same amounts of the ligand are used. Kinetics, i.e., an association rate constant (ka) and a dissociation rate constant (kd), can be determined from the curve of the sensorgram, while affinity (KD) can be determined from the ratio between these constants. Inhibition assay is also preferably used in the BIACORE method. Examples of the inhibition assay are described in Proc. Natl. Acad. Sci. USA (2006) 103 (11), 4005-4010.

### Does not Bind to CD3 and CD137 (4-1BB) at the Same Time

The term “does not bind to CD3 and CD137 (4-1BB) at the same time” or “does not bind to CD3 and CD137 (4-1BB) simultaneously” means that the antigen-binding moiety or antibody variable region of the present disclosure cannot bind to CD137 in a state bound with CD3 whereas the antigen-binding moiety or antibody variable region cannot bind to CD3 in a state bound with CD137. In this context, the phrase “not bind to CD3 and CD137 at the same time” also includes not cross-linking a cell expressing CD3 to a cell expressing CD137, or not binding to CD3 and CD137 each expressed on a different cell, at the same time. This phrase further includes the case where the variable region is capable of binding to both CD3 and CD137 at the same time when CD3 and CD137 are not expressed on cell membranes, as with soluble proteins, or both reside on the same cell, but cannot bind to CD3 and CD137 each expressed on a different cell, at the same time. Such an antibody variable region is not particularly limited as long as the antibody variable region has these functions. Examples thereof can include variable regions derived from an IgG-type antibody variable region by the alteration of a portion of its amino acids so as to bind to the desired antigen. The amino acid to be altered is selected from, for example, amino acids whose alteration does not cancel the binding to the antigen, in an antibody variable region binding to CD3 or CD137.

In this context, the phrase “expressed on different cells” merely means that the antigens are expressed on separate cells. The combination of such cells may be, for example, the same types of cells such as a T cell and another T cell, or may be different types of cells such as a T cell and an NK cell.

Whether the antigen-binding molecule of the present disclosure does “not bind to CD3 and CD137 at the same time” can be confirmed by: confirming the antigen-binding molecule to have binding activity against both CD3 and CD137; then allowing either CD3 or CD137 to bind in advance to the antigen-binding molecule comprising the variable region having this binding activity; and then determining the presence or absence of its binding activity against the other one by the method mentioned above. Alternatively, this can also be confirmed by determining whether the binding of the antigen-binding molecule to either CD3 or CD137 immobilized on an ELISA plate or a sensor chip is inhibited by the addition of the other one into the solution. In some embodiments, the binding of the antigen-binding molecule of the present disclosure to either CD3 or CD137 is inhibited by binding of the antigen-binding molecule to the other by at least 50%, preferably 60% or more, more preferably 70% or more, more preferably 80% or more, further preferably 90% or more, or even more preferably 95% or more.

In one aspect, while one antigen (e.g. CD3) is immobilized, the inhibition of the binding of the antigen-binding molecule to CD3 can be determined in the presence of the other antigen (e.g. CD137) by methods known in prior art (i.e. ELISA, BIACORE™, and so on). In another aspect, while CD137 is immobilized, the inhibition of the binding of the antigen-binding molecule to CD137 also can be determined in the presence of CD3. When either one of two aspects mentioned above is conducted, the antigen-binding molecule of the present disclosure is determined not to bind to CD3 and CD137 at the same time if the binding is inhibited by at least 50%, preferably 60% or more, preferably 70% or more, further preferably 80% or more, further preferably 90% or more, or even more preferably 95% or more.

In some embodiments, the concentration of the antigen injected as an analyte is at least 1-fold, 2-fold, 5-fold, 10-fold, 30-fold, 50-fold, or 100-fold higher than the concentration of the other antigen to be immobilized.

In preferable manner, the concentration of the antigen injected as an analyte is 100-fold higher than the concentration of the other antigen to be immobilized and the binding is inhibited by at least 80%.

In one embodiment, the ratio of the KD value for the CD3 (analyte)-binding activity of the antigen-binding molecule to the CD137 (immobilized)-binding activity of the antigen-binding molecule (KD (CD3)/KD (CD137)) is calculated and the CD3 (analyte) concentration which is 10-fold, 50-fold, 100-fold, or 200-fold of the ratio of the KD value (KD(CD3)/KD(CD137) higher than the CD137 (immobilized) concentration can be used for the competition measurement above. (e.g. 1-fold, 5-fold, 10-fold, or 20-fold higher concentration can be selected when the ratio of the KD value is 0.1. Furthermore, 100-fold, 500-fold, 1000-fold, or 2000-fold higher concentration can be selected when the ratio of the KD value is 10.)

In one aspect, while one antigen (e.g. CD3) is immobilized, the attenuation of the binding signal of the antigen-binding molecule to CD3 can be determined in the presence of the other antigen (e.g. CD137) by methods known in prior art (i.e. ELISA, ECL and so on). In another aspect, while CD137 is immobilized, the attenuation of the binding signal of the antigen-binding molecule to CD137 also can be determined in the presence of CD3. When either one of two aspects mentioned above is conducted, the antigen-binding molecule of the present disclosure is determined not to bind to CD3 and CD137 at the same time if the binding signal is attenuated by at least 50%, preferably 60% or more, preferably 70% or more, further preferably 80% or more, further preferably 90% or more, or even more preferably 95% or more.

In some embodiments, the concentration of the antigen injected as an analyte is at least 1-fold, 2-fold, 5-fold, 10-fold, 30-fold, 50-fold, or 100-fold higher than the concentration of the other antigen to be immobilized.

In preferable manner, the concentration of the antigen injected as an analyte is 100-fold higher than the concentration of the other antigen to be immobilized and the binding is inhibited by at least 80%.

In one embodiment, the ratio of the KD value for the CD3 (analyte)-binding activity of the antigen-binding molecule to the CD137 (immobilized)-binding activity of the antigen-binding molecule (KD (CD3)/KD (CD137)) is calculated and the CD3 (analyte) concentration which is 10-fold, 50-fold, 100-fold, or 200-fold of the ratio of the KD value (KD(CD3)/KD(CD137) higher than the CD137 (immobilized) concentration can be used for the measurement above. (e.g. 1-fold, 5-fold, 10-fold, or 20-fold higher concentration can be selected when the ratio of the KD value is 0.1. Furthermore, 100-fold, 500-fold, 1000-fold, or 2000-fold higher concentration can be selected when the ratio of the KD value is 10.)

Specifically, in the case of using, for example, the ECL method, a biotin-labeled antigen-binding molecule to be tested, CD3 labeled with sulfo-tag (Ru complex), and an unlabeled CD137 are prepared. When the antigen-binding molecule to be tested is capable of binding to CD3 and CD137, but does not bind to CD3 and CD137 at the same time, the luminescence signal of the sulfo-tag is detected in the absence of the unlabeled CD137 by adding the mixture of the antigen-binding molecule to be tested and labeled CD3 onto a streptavidin-immobilized plate, followed by light development. By contrast, the luminescence signal is decreased in the presence of unlabeled CD137. This decrease in luminescence signal can be quantified to determine relative binding activity. This analysis may be similarly conducted using the labeled CD137 and the unlabeled CD3.

In the case of the ALPHAScreen, the antigen-binding molecule to be tested interacts with CD3 in the absence of the competing CD137 to generate signals of 520 to 620 nm. The untagged CD137 competes with CD3 for the interaction with the antigen-binding molecule to be tested. Decrease in fluorescence caused as a result of the competition can be quantified to thereby determine relative binding activity. The polypeptide biotinylation using sulfo-NHS-biotin or the like is known in the art. CD3 can be tagged with GST by an appropriately adopted method which involves, for example: fusing a polynucleotide encoding CD3 in flame with a polynucleotide encoding GST; and allowing the resulting fusion gene to be expressed by cells or the like harboring vectors capable of expression thereof, followed by purification using a glutathione column. The obtained signals are preferably analyzed using, for example, software GRAPHPAD PRISM (GraphPad Software, Inc., San Diego) adapted to a one-site competition model based on nonlinear regression analysis. This analysis may be similarly conducted using the tagged CD137 and the untagged CD3.

Alternatively, a method using fluorescence resonance energy transfer (FRET) may be used. FRET is a phenomenon in which excitation energy is transferred directly between two fluorescent molecules located in proximity to each other by electron resonance. When FRET occurs, the excitation energy of a donor (fluorescent molecule having an excited state) is transferred to an acceptor (another fluorescent molecule located near the donor) so that the fluorescence emitted from the donor disappears (to be precise, the lifetime of the fluorescence is shortened) and instead, the fluorescence is emitted from the acceptor. By use of this phenomenon, whether or not bind to CD3 and CD137 at the same time can be analyzed. For example, when CD3 carrying a fluorescence donor and CD137 carrying a fluorescence acceptor bind to the antigen-binding molecule to be tested at the same time, the fluorescence of the donor disappears while the fluorescence is emitted from the acceptor. Therefore, change in fluorescence wavelength is observed. Such an antibody is confirmed to bind to CD3 and CD137 at the same time. On the other hand, if the mixing of CD3, CD137, and the antigen-binding molecule to be tested does not change the fluorescence wavelength of the fluorescence donor bound with CD3, this antigen-binding molecule to be tested can be regarded as antigen binding domain that is capable of binding to CD3 and CD137, but does not bind to CD3 and CD137 at the same time.

For example, a biotin-labeled antigen-binding molecule to be tested is allowed to bind to streptavidin on the donor bead, while CD3 tagged with glutathione S transferase (GST) is allowed to bind to the acceptor bead. The antigen-binding molecule to be tested interacts with CD3 in the absence of the competing second antigen to generate signals of 520 to 620 nm. The untagged second antigen competes with CD3 for the interaction with the antigen-binding molecule to be tested. Decrease in fluorescence caused as a result of the competition can be quantified to thereby determine relative binding activity. The polypeptide biotinylation using sulfo-NHS-biotin or the like is known in the art. CD3 can be tagged with GST by an appropriately adopted method which involves, for example: fusing a polynucleotide encoding CD3 in flame with a polynucleotide encoding GST; and allowing the resulting fusion gene to be expressed by cells or the like harboring vectors capable of expression thereof, followed by purification using a glutathione column. The obtained signals are preferably analyzed using, for example, software GRAPHPAD PRISM (GraphPad Software, Inc., San Diego) adapted to a one-site competition model based on nonlinear regression analysis.

The tagging is not limited to the GST tagging and may be carried out with any tag such as, but not limited to, a histidine tag, MBP, CBP, a Flag tag, an HA tag, a V5 tag, or a c-myc tag. The binding of the antigen-binding molecule to be tested to the donor bead is not limited to the binding using biotin-streptavidin reaction. Particularly, when the antigen-binding molecule to be tested comprises Fc, a possible method involves allowing the antigen-binding molecule to be tested to bind via an Fc-recognizing protein such as protein A or protein G on the donor bead.

Also, the case where the variable region is capable of binding to CD3 and CD137 at the same time when CD3 and CD137 are not expressed on cell membranes, as with soluble proteins, or both reside on the same cell, but cannot bind to CD3 and CD137 each expressed on a different cell, at the same time can also be assayed by a method known in the art.

Specifically, the antigen-binding molecule to be tested has been confirmed to be positive in ECL-ELISA for detecting binding to CD3 and CD137 at the same time is also mixed with a cell expressing CD3 and a cell expressing CD137. The antigen-binding molecule to be tested can be shown to be incapable of binding to CD3 and CD137 expressed on different cells, at the same time unless the antigen-binding molecule and these cells bind to each other at the same time. This assay can be conducted by, for example, cell-based ECL-ELISA. The cell expressing CD3 is immobilized onto a plate in advance. After binding of the antigen-binding molecule to be tested thereto, the cell expressing CD137 is added to the plate. A different antigen expressed only on the cell expressing CD137 is detected using a sulfo-tag-labeled antibody against this antigen. A signal is observed when the antigen-binding molecule binds to the two antigens respectively expressed on the two cells, at the same time. No signal is observed when the antigen-binding molecule does not bind to these antigens at the same time.

Alternatively, this assay may be conducted by the ALPHAScreen method. The antigen-binding molecule to be tested is mixed with a cell expressing CD3 bound with the donor bead and a cell expressing CD137 bound with the acceptor bead. A signal is observed when the antigen-binding molecule binds to the two antigens expressed on the two cells respectively, at the same time. No signal is observed when the antigen-binding molecule does not bind to these antigens at the same time.

Alternatively, this assay may also be conducted by an Octet interaction analysis method. First, a cell expressing CD3 tagged with a peptide tag is allowed to bind to a biosensor that recognizes the peptide tag. A cell expressing CD137 and the antigen-binding molecule to be tested are placed in wells and analyzed for interaction. A large wavelength shift caused by the binding of the antigen-binding molecule to be tested and the cell expressing CD137 to the biosensor is observed when the antigen-binding molecule binds to the two antigens expressed on the two cells respectively, at the same time. A small wavelength shift caused by the binding of only the antigen-binding molecule to be tested to the biosensor is observed when the antigen-binding molecule does not bind to these antigens at the same time.

Instead of these methods based on the binding activity, assay based on biological activity may be conducted. For example, a cell expressing CD3 and a cell expressing CD137 are mixed with the antigen-binding molecule to be tested, and cultured. The two antigens expressed on the two cells respectively are mutually activated via the antigen-binding molecule to be tested when the antigen-binding molecule binds to these two antigens at the same time. Therefore, change in activation signal, such as increase in the respective downstream phosphorylation levels of the antigens, can be detected. Alternatively, cytokine production is induced as a result of the activation. Therefore, the amount of cytokines produced can be measured to thereby confirm whether or not to bind to the two cells at the same time. Alternatively, cytotoxicity against a cell expressing CD137 is induced as a result of the activation. Alternatively, the expression of a reporter gene is induced by a promoter which is activated at the downstream of the signal transduction pathway of CD137 or CD3 as a result of the activation. Therefore, the cytotoxicity or the amount of reporter proteins produced can be measured to thereby confirm whether or not to bind to the two cells at the same time.

### Fab Molecule

A “Fab molecule” refers to a protein consisting of the VH and CH1 domain of the heavy chain (the “Fab heavy chain”) and the VL and CL domain of the light chain (the “Fab light chain”) of an immunoglobulin.

### Fused

By “fused” is meant that the components (e.g. a Fab molecule and an Fc domain subunit) are linked by peptide bonds, either directly or via one or more peptide linkers.

### “Crossover” Fab

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

### “Conventional” Fab

In contrast thereto, by a “conventional” Fab molecule is meant a Fab molecule in its natural format, i.e. comprising a heavy chain composed of the heavy chain variable and constant regions (VH-CH1), and a light chain composed of the light chain variable and constant regions (VL-CL). The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called alpha (IgA), delta (IgD), epsilon (IgE), gamma (IgG), or mu (IgM), some of which may be further divided into subtypes, e.g. gamma 1 (IgG1), gamma 2 (IgG2), gamma 3 (IgG3), gamma 4 (IgG4), alpha 1 (IgA1) and alpha 2 (IgA2). The light chain of an immunoglobulin may be assigned to one of two types, called kappa and lambda, based on the amino acid sequence of its constant domain. An immunoglobulin essentially consists of two Fab molecules and an Fc domain, linked via the immunoglobulin hinge region.

### Affinity

“Affinity” refers to the strength of the sum total of noncovalent interactions between a single binding site of a molecule (e.g., an antigen-binding molecule or antibody) and its binding partner (e.g., an antigen). Unless indicated otherwise, as used herein, “binding affinity” refers to intrinsic binding affinity which reflects a 1:1 interaction between members of a binding pair (e.g., antigen-binding molecule and antigen, or antibody and antigen). The affinity of a molecule X for its partner Y can generally be represented by the dissociation constant (KD), which is the ratio of dissociation and association rate constants (koff and kon, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

### Methods to Determine Affinity

In certain embodiments, the antigen-binding molecule or antibody provided herein has a dissociation constant (KD) of 1 micro M or less, 120 nM or less, 100 nM or less, 80 nM or less, 70 nM or less, 50 nM or less, 40 nM or less, 30 nM or less, 20 nM or less, 10 nM or less, 2 nM or less, 1 nM or less, 0.1 nM or less, 0.01 nM or less, or 0.001 nM or less (e.g., 10⁻⁸ M or less, 10⁻⁸ M to 10⁻¹³M, 10⁻⁹ M to 10⁻¹³ M) for its antigen. In certain embodiments, the KD value of the antibody/antigen-binding molecule for CD3, CD137 or CLDN6 falls within the range of 1-40, 1-50, 1-70, 1-80, 30-50, 30-70, 30-80, 40-70, 40-80, or 60-80 nM.

In one embodiment, KD is measured by a radiolabeled antigen-binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro 1/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE (registered trademark)-3000 (BIAcore, Inc., Piscataway, N.J.) is performed at 25 degrees C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 micro g/ml (˜0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

According to the methods for measuring the affinity of the antigen-binding molecule or the antibody described above, persons skilled in art can carry out affinity measurement for other antigen-binding molecules or antibodies, towards various kind of antigens.

### Antibody

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

### Class of Antibody

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

Unless otherwise indicated, amino acid residues in the light chain constant region are numbered herein according to Kabat et al., and numbering of amino acid residues in the heavy chain constant region is according to the EU numbering system, also called the EU index, as described in Kabat et al., Sequences of Proteins of Immunological Interest, 5th Ed. Public Health Service, National Institutes of Health, Bethesda, Md., 1991.

### Framework

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

### Human Consensus Framework

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

### Chimeric Antibody

The term “chimeric” antibody refers to an antibody in which a portion of the heavy and/or light chain is derived from a particular source or species, while the remainder of the heavy and/or light chain is derived from a different source or species. Similarly, the term “chimeric antibody variable domain” refers to an antibody variable region in which a portion of the heavy and/or light chain variable region is derived from a particular source or species, while the remainder of the heavy and/or light chain variable region is derived from a different source or species.

### Humanized Antibody

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A “humanized antibody variable region” refers to the variable region of a humanized antibody.

### Human Antibody

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “human antibody variable region” refers to the variable region of a human antibody.

### Polynucleotide (Nucleic Acid)

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

### Isolated (Nucleic Acid)

An “isolated” nucleic acid molecule is one which has been separated from a component of its natural environment. An isolated nucleic acid molecule further includes a nucleic acid molecule contained in cells that ordinarily contain the nucleic acid molecule, but the nucleic acid molecule is present extrachromosomally or at a chromosomal location that is different from its natural chromosomal location.

### Vector

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Vectors could be introduced into host cells using virus or electroporation. However, introduction of vectors is not limited to in vitro method. For example, vectors could also be introduced into a subject using in vivo method directly.

### Host Cell

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### Specificity

“Specific” means that a molecule that binds specifically to one or more binding partners does not show any significant binding to molecules other than the partners. Furthermore, “specific” is also used when an antigen-binding site is specific to a particular epitope of multiple epitopes contained in an antigen. If an antigen-binding molecule binds specifically to an antigen, it is also described as “the antigen-binding molecule has/shows specificity to/towards the antigen”. When an epitope bound by an antigen-binding site is contained in multiple different antigens, an antigen-binding molecule containing the antigen-binding site can bind to various antigens that have the epitope.

### Antibody Fragment

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

### Variable Fragment (Fv)

Herein, the term “variable fragment (Fv)” refers to the minimum unit of an antibody-derived antigen-binding site that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen.

scFv, Single-Chain Antibody, and Sc(Fv)

Herein, the terms “scFv”, “single-chain antibody”, and “sc(Fv)₂” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen-binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

An scFv is an single chain low molecule weight antibody in which VH and VL forming Fv are linked together by a peptide linker (Proc. Natl. Acad. Sci. U.S.A. (1988) 85(16), 5879-5883). VH and VL can be retained in close proximity by the peptide linker.

sc(Fv)₂ is a single chain antibody in which four variable regions of two VL and two VH are linked by linkers such as peptide linkers to form a single chain (J Immunol. Methods (1999) 231(1-2), 177-189). The two VH and two VL may be derived from different monoclonal antibodies. Such sc(Fv)₂ preferably includes, for example, a bispecific sc(Fv)₂ that recognizes two epitopes present in a single antigen as disclosed in the Journal of Immunology (1994) 152(11), 5368-5374. sc(Fv)₂ can be produced by methods known to those skilled in the art. For example, sc(Fv)₂ can be produced by linking scFv by a linker such as a peptide linker.

Herein, an sc(Fv)₂ includes two VH units and two VL units which are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Examples of the form are listed below. [VL]-linker-[VH]-linker-[VH]-linker-[VL] [VH]-linker-[VL]-linker-[VL]-linker-[VH] [VH]-linker-[VH]-linker-[VL]-linker-[VL] [VL]-linker-[VL]-linker-[VH]-linker-[VH] [YL]-linker-[VH]-linker-[YL]-linker-[VH]

The molecular form of sc(Fv)₂ is also described in detail in WO 2006/132352. According to these descriptions, those skilled in the art can appropriately prepare desired sc(Fv)₂ to produce the polypeptide complexes disclosed herein.

Furthermore, the antigen-binding molecules or antibodies of the present disclosure may be conjugated with a carrier polymer such as PEG or an organic compound such as an anticancer agent. Alternatively, a sugar chain addition sequence is preferably inserted into the antigen-binding molecules or antibodies such that the sugar chain produces a desired effect.

The linkers to be used for linking the variable regions of an antibody comprise arbitrary peptide linkers that can be introduced by genetic engineering, synthetic linkers, and linkers disclosed in, for example, Protein Engineering, 9(3), 299-305, 1996. However, peptide linkers are preferred in the present disclosure. The length of the peptide linkers is not particularly limited, and can be suitably selected by those skilled in the art according to the purpose. The length is preferably five amino acids or more (without particular limitation, the upper limit is generally 30 amino acids or less, preferably 20 amino acids or less), and particularly preferably 15 amino acids. When sc(Fv)₂ contains three peptide linkers, their length may be all the same or different.

For example, such peptide linkers include:

Ser,
Gly-Ser,
Gly-Gly-Ser,
Ser-Gly-Gly,
(SEQ ID NO: 171)
Gly-Gly-Gly-Ser,
(SEQ ID NO: 172)
Ser-Gly-Gly-Gly,
(SEQ ID NO: 173)
Gly-Gly-Gly-Gly-Ser,
(SEQ ID NO: 174)
Ser-Gly-Gly-Gly-Gly,
(SEQ ID NO: 175)
Gly-Gly-Gly-Gly-Gly-Ser,
(SEQ ID NO: 176)
Ser-Gly-Gly-Gly-Gly-Gly,
(SEQ ID NO: 177)
Gly-Gly-Gly-Gly-Gly-Gly-Ser,
(SEQ ID NO: 178)
Ser-Gly-Gly-Gly-Gly-Gly-Gly,
(Gly-Gly-Gly-Gly-Ser (SEQ ID NO: 173))n,
and
(Ser-Gly-Gly-Gly-Gly (SEQ ID NO: 174))n,

where n is an integer of 1 or larger. The length or sequences of peptide linkers can be selected accordingly by those skilled in the art depending on the purpose.

Synthetic linkers (chemical crosslinking agents) are routinely used to crosslink peptides, and examples include:

N-hydroxy succinimide (NHS),
disuccinimidyl suberate (DSS),
bis(sulfosuccinimidyl) suberate (BS3),
dithiobis(succinimidyl propionate) (DSP),
dithiobis(sulfosuccinimidyl propionate) (DTSSP),
ethylene glycol bis(succinimidyl succinate) (EGS),
ethylene glycol bis(sulfosuccinimidyl succinate) (sulfo-EGS),
disuccinimidyl tartrate (DST), disulfosuccinimidyl tartrate (sulfo-DST),
bis[2-(succinimidoxycarbonyloxy)ethyl] sulfone (BSOCOES), and
bis[2-(sulfosuccinimidoxycarbonyloxy)ethyl] sulfone (sulfo-BSOCOES). These crosslinking agents are commercially available.

In general, three linkers are required to link four antibody variable regions together. The linkers to be used may be of the same type or different types.

### Fab, F(ab′)₂, and Fab′

“Fab” consists of a single light chain, and a CH1 domain and variable region from a single heavy chain. The heavy chain of Fab molecule cannot form disulfide bonds with another heavy chain molecule.

“F(ab′)₂” or “Fab” is produced by treating an immunoglobulin (monoclonal antibody) with a protease such as pepsin and papain, and refers to an antibody fragment generated by digesting an immunoglobulin (monoclonal antibody) near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)₂” consists of two light chains and two heavy chains comprising the constant region of a CH1 domain and a portion of CH2 domains so that disulfide bonds are formed between the two heavy chains. The F(ab′)₂ disclosed herein can be preferably produced as follows. A whole monoclonal antibody or such comprising a desired antigen-binding site is partially digested with a protease such as pepsin; and Fc fragments are removed by adsorption onto a Protein A column. The protease is not particularly limited, as long as it can cleave the whole antibody in a selective manner to produce F(ab′)₂ under an appropriate setup enzyme reaction condition such as pH. Such proteases include, for example, pepsin and ficin.

### Fc Region

The term “Fc region” or “Fc domain” refers to a region comprising a fragment consisting of a hinge or a portion thereof and CH2 and CH3 domains in an antibody molecule. The Fc region of IgG class means, but is not limited to, a region from, for example, cysteine 226 (EU numbering (also referred to as EU index herein)) to the C terminus or proline 230 (EU numbering) to the C terminus. The Fc region can be preferably obtained by the partial digestion of, for example, an IgG1, IgG2, IgG3, or IgG4 monoclonal antibody with a proteolytic enzyme such as pepsin followed by the re-elution of a fraction adsorbed on a protein A column or a protein G column. Such a proteolytic enzyme is not particularly limited as long as the enzyme is capable of digesting a whole antibody to restrictively form Fab or F(ab′)2 under appropriately set reaction conditions (e.g., pH) of the enzyme. Examples thereof can include pepsin and papain.

An Fc region derived from, for example, naturally occurring IgG can be used as the “Fc region” of the present disclosure. In this context, the naturally occurring IgG means a polypeptide that contains an amino acid sequence identical to that of IgG found in nature and belongs to a class of an antibody substantially encoded by an immunoglobulin gamma gene. The naturally occurring human IgG means, for example, naturally occurring human IgG1, naturally occurring human IgG2, naturally occurring human IgG3, or naturally occurring human IgG4. The naturally occurring IgG also includes variants or the like spontaneously derived therefrom. A plurality of allotype sequences based on gene polymorphism are described as the constant regions of human IgG1, human IgG2, human IgG3, and human IgG4 antibodies in Sequences of proteins of immunological interest, NIH Publication No. 91-3242, any of which can be used in the present disclosure. Particularly, the sequence of human IgG1 may have DEL or EEM as an amino acid sequence of EU numbering positions 356 to 358.

In some embodiments, the Fc domain of the multispecific antigen-binding molecule consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the multispecific antigen-binding molecule described herein comprises not more than one Fc domain.

In one embodiment described herein, the Fc domain of the multispecific-antigen binding molecule is an IgG Fc domain. In a particular embodiment, the Fc domain is an IgG1 Fc domain. In another embodiment, the Fc domain is an IgG1 Fc domain. In a further particular embodiment, the Fc domain is a human IgG1 Fc region.

In one aspect, the present disclosure provides a multispecific antigen-binding molecule further comprising
(iii) an Fc domain which exhibits reduced binding affinity to human Fc gamma receptor, as compared to a native human IgG1 Fc domain,
   wherein the Fc domain is composed of a first Fc-region subunit and a second Fc-region subunit that are capable of stable association.

In one aspect, the present disclosure provides a multispecific antigen-binding molecule further comprising
(iii) an Fc domain which exhibits reduced binding affinity to human Fc gamma receptor, as compared to a native human IgG1 Fc domain,
   wherein the Fc domain comprises (e1) or (e2) below:
(e1) the first Fc-region subunit comprising Cys at position 349, Ser at position 366, Ala at position 368 and Val at position 407, and the second Fc-region comprising Cys at position 354 and Trp at position 366;
(e2) the first Fc-region subunit comprising Glu at position 439, and the second Fc-region comprising Lys at position 356;
   wherein the amino acid positions are numbered according to EU index.

In one aspect, the present disclosure provides a multispecific antigen-binding molecule further comprising
(iii) an Fc domain which exhibits reduced binding affinity to human Fc gamma receptor, as compared to a native human IgG1 Fc domain,
   wherein the first and/or the second Fc-region subunit comprised in the Fc domain comprises (f1) or (f2) below:
   (f1) Ala at position 234 and Ala at position 235;
   (f2) Ala at position 234, Ala at position 235 and Ala at position 297;
   wherein the amino acid positions are numbered according to EU index.

In one aspect, the present disclosure provides a multispecific antigen-binding molecule further comprising
(iii) an Fc domain which exhibits reduced binding affinity to human Fc gamma receptor, as compared to a native human IgG1 Fc domain,
   wherein the Fc domain further exhibits stronger FcRn binding affinity to human FcRn, as compared to a native human IgG1 Fc domain.

In one aspect, the present disclosure provides a multispecific antigen-binding molecule further comprising
(iii) an Fc domain which exhibits reduced binding affinity to human Fc gamma receptor, as compared to a native human IgG1 Fc domain,
   wherein the first and/or the second Fc region subunit comprised in the Fc domain comprises Leu at position 428, Ala at position 434, Arg at position 438, and Glu at position 440,
   wherein the amino acid positions are numbered according to EU index.
      Fc Region with a Reduced Fc Receptor (Fc Gamma Receptor)-Binding Activity

In certain embodiments, the Fc domain of the multispecific antigen-binding molecules described herein exhibits reduced binding affinity to an Fc receptor, as compared to a native IgG1 Fc domain. In one such embodiment the Fc domain (or the multispecific antigen-binding molecule comprising said Fc domain) exhibits less than 50%, preferably less than 20%, more preferably less than 10% and most preferably less than 5% of the binding affinity to an Fc receptor, as compared to a native IgG1 Fc domain (or a multispecific antigen-binding molecule comprising a native IgG1 Fc domain). In one embodiment, the Fc domain (or the multispecific antigen-binding molecule comprising said Fc domain) does not substantially bind to an Fc receptor. In a particular embodiment, the Fc receptor is an Fc gamma receptor. In one embodiment the Fc receptor is a human Fc receptor. In one embodiment, the Fc receptor is an activating Fc receptor. In a specific embodiment the Fc receptor is an activating human Fc gamma receptor, more specifically human Fc gamma RIIIa, Fc gamma RI or Fc gamma RIIa, most specifically human Fc gamma RIIIa.

In certain embodiments, the Fc domain of the multispecific antigen-binding molecule comprises one or more amino acid mutation that reduces the binding affinity of the Fc domain to an Fc receptor. Typically, the same one or more amino acid mutation is present in each of the two subunits of the Fc domain. In one embodiment the amino acid mutation reduces the binding affinity of the Fc domain to an Fc receptor. In one embodiment, the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the multispecific antigen-binding molecule comprising an engineered Fc domain exhibits less than 20%, particularly less than 10%, more particularly less than 5% of the binding affinity to an Fc receptor as compared to a multispecific antigen-binding molecule comprising a non-engineered Fc domain. In a particular embodiment the Fc receptor is an Fc gamma receptor. In some embodiments, the Fc receptor is a human Fc receptor. In some embodiments the Fc receptor is an activating Fc receptor. In a specific embodiment, the Fc receptor is an activating human Fc gamma receptor, more specifically human Fc gamma RIIIa, Fc gamma RI or Fc gamma RIIa, most specifically human Fc gamma RIIIa. Preferably, binding to each of these receptors is reduced.

In one embodiment, the amino acid mutation that reduces the binding affinity of the Fc domain to an Fc receptor is an amino acid substitution. In one embodiment, the Fc domain comprises an amino acid substitution at a position selected from the group of E233, L234, L235, N297, P331 and P329. In a more specific embodiment, the Fc domain comprises an amino acid substitution at a position selected from the group of L234, L235 and P329. In some embodiments, the Fc domain comprises the amino acid substitutions L234A and L235A. In one such embodiment, the Fc domain is an IgG1 Fc domain, particularly a human IgG1 Fc domain. In one embodiment, the Fc domain comprises an amino acid substitution at position P329. In a more specific embodiment, the amino acid substitution is P329A or P329G, particularly P329G. In one embodiment the Fc domain comprises an amino acid substitution at position P329 and a further amino acid substitution at a position selected from E233, L234, L235, N297 and P331. In a more specific embodiment, the further amino acid substitution is E233P, L234A, L235A, L235E, N297A, N297D or P331S. In particular embodiments, the Fc domain comprises amino acid substitutions at positions P329, L234 and L235. In more particular embodiments the Fc domain comprises the amino acid mutations L234A, L235A and P329G (“P329G LALA”). In one such embodiment, the Fc domain is an IgG1 Fc domain, particularly a human IgG1 Fc domain. The “P329G LALA” combination of amino acid substitutions almost completely abolishes Fc gamma receptor (as well as complement) binding of a human IgG1 Fc domain, as described in PCT publication no. WO 2012/130831. WO 2012/130831 also describes methods of preparing such mutant Fc domains and methods for determining its properties such as Fc receptor binding or effector functions.

IgG4 antibodies exhibit reduced binding affinity to Fc receptors and reduced effector functions as compared to IgG1 antibodies. Hence, in some embodiments, the Fc domain of the T cell activating bispecific antigen binding molecules described herein is an IgG4 Fc domain, particularly a human IgG4 Fc domain. In one embodiment, the IgG4 Fc domain comprises amino acid substitutions at position 5228, specifically the amino acid substitution S228P. To further reduce its binding affinity to an Fc receptor and/or its effector function, in one embodiment the IgG4 Fc domain comprises an amino acid substitution at position L235, specifically the amino acid substitution L235E. In another embodiment, the IgG4 Fc domain comprises an amino acid substitution at position P329, specifically the amino acid substitution P329G. In a particular embodiment, the IgG4 Fc domain comprises amino acid substitutions at positions S228, L235 and P329, specifically amino acid substitutions S228P, L235E and P329G. Such IgG4 Fc domain mutants and their Fc gamma receptor binding properties are described in PCT publication no. WO 2012/130831.

In certain embodiments, N-glycosylation of the Fc domain has been eliminated. In one such embodiment, the Fc domain comprises an amino acid mutation at position N297, particularly an amino acid substitution replacing asparagine by alanine (N297A) or aspartic acid (N297D).

In a particular preferred embodiment, the Fc domain exhibiting reduced binding affinity to an Fc receptor, as compared to a native IgG1 Fc domain, is a human IgG1 Fc domain comprising the amino acid substitutions L234A, L235A and N297A.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing Fc gamma Ina receptor.

### Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half-life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

### Fc Gamma Receptor

Fc gamma receptor refers to a receptor capable of binding to the Fc domain of monoclonal IgG1, IgG2, IgG3, or IgG4 antibodies, and includes all members belonging to the family of proteins substantially encoded by an Fc gamma receptor gene. In human, the family includes Fc gamma RI (CD64) including isoforms Fc gamma RIa, Fc gamma RIb and Fc gamma RIc; Fc gamma RII (CD32) including isoforms Fc gamma RIIa (including allotype H131 and R131), Fc gamma RIIb (including Fc gamma RIIb-1 and Fc gamma RIIb-2), and Fc gamma RIIc; and Fc gamma RIII (CD16) including isoform Fc gamma RIIIa (including allotype V158 and F158) and Fc gamma RIIIb (including allotype Fc gamma RIIIb-NA1 and Fc gamma RIIIb-NA2); as well as all unidentified human Fc gamma receptors, Fc gamma receptor isoforms, and allotypes thereof. However, Fc gamma receptor is not limited to these examples. Without being limited thereto, Fc gamma receptor includes those derived from humans, mice, rats, rabbits, and monkeys. Fc gamma receptor may be derived from any organisms. Mouse Fc gamma receptor includes, without being limited to, Fc gamma RI (CD64), Fc gamma RII (CD32), Fc gamma RIB (CD16), and Fc gamma RIII-2 (CD16-2), as well as all unidentified mouse Fc gamma receptors, Fc gamma receptor isoforms, and allotypes thereof. Such preferred Fc gamma receptors include, for example, human Fc gamma RI (CD64), Fc gamma RIIA (CD32), Fc gamma RIIB (CD32), Fc gamma RIIIA (CD16), and/or Fc gamma RIIIB (CD16). The polynucleotide sequence and amino acid sequence of Fc gamma RI are shown in RefSeq accession number NM_000566.3 and RefSeq accession number NP_000557.1, respectively; the polynucleotide sequence and amino acid sequence of Fc gamma RIIA are shown in RefSeq accession number BC020823.1 and RefSeq accession number AAH20823.1, respectively; the polynucleotide sequence and amino acid sequence of Fc gamma RIIB are shown in RefSeq accession number BC146678.1 and RefSeq accession number AAI46679.1, respectively; the polynucleotide sequence and amino acid sequence of Fc gamma RIIIA are shown in RefSeq accession number BC033678.1 and RefSeq accession number AAH33678.1, respectively; and the polynucleotide sequence and amino acid sequence of Fc gamma RIIIB are shown in RefSeq accession number BC128562.1 and RefSeq accession number AAI28563.1, respectively. Whether an Fc gamma receptor has binding activity to the Fc domain of a monoclonal IgG1, IgG2, IgG3, or IgG4 antibody can be assessed by ALPHA screen (Amplified Luminescent Proximity Homogeneous Assay), surface plasmon resonance (SPR)-based BIACORE™ method, and others (Proc. Natl. Acad. Sci. USA (2006) 103(11), 4005-4010), in addition to the above-described FACS and ELISA formats.

Meanwhile, “Fc ligand” or “effector ligand” refers to a molecule and preferably a polypeptide that binds to an antibody Fc domain, forming an Fc/Fc ligand complex. The molecule may be derived from any organisms. The binding of an Fc ligand to Fc preferably induces one or more effector functions. Such Fc ligands include, but are not limited to, Fc receptors, Fc gamma receptor, Fc alpha receptor, Fc beta receptor, FcRn, C1q, and C3, mannan-binding lectin, mannose receptor, Staphylococcus Protein A, Staphylococcus Protein G, and viral Fc gamma receptors. The Fc ligands also include Fc receptor homologs (FcRH) (Davis et al., (2002) Immunological Reviews 190, 123-136), which are a family of Fc receptors homologous to Fc gamma receptor. The Fc ligands also include unidentified molecules that bind to Fc.

### Fc Gamma Receptor-Binding Activity

The impaired binding activity of Fc domain to any of the Fc gamma receptors Fc gamma RI, Fc gamma RIIA, Fc gamma RIIB, Fc gamma RIIIA, and/or Fc gamma RIIIB can be assessed by using the above-described FACS and ELISA formats as well as ALPHA screen (Amplified Luminescent Proximity Homogeneous Assay) and surface plasmon resonance (SPR)-based BIACORE method (Proc. Natl. Acad. Sci. USA (2006) 103(11), 4005-4010).

ALPHA screen is performed by the ALPHA technology based on the principle described below using two types of beads: donor and acceptor beads. A luminescent signal is detected only when molecules linked to the donor beads interact biologically with molecules linked to the acceptor beads and when the two beads are located in close proximity Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled antigen-binding molecule or antibody is immobilized to the donor beads and glutathione S-transferase (GST)-tagged Fc gamma receptor is immobilized to the acceptor beads. In the absence of an antigen-binding molecule or antibody comprising a competitive mutant Fc domain, Fc gamma receptor interacts with an antigen-binding molecule or antibody comprising a wild-type Fc domain, inducing a signal of 520 to 620 nm as a result. The antigen-binding molecule or antibody having a non-tagged mutant Fc domain competes with the antigen-binding molecule or antibody comprising a wild-type Fc domain for the interaction with Fc gamma receptor. The relative binding affinity can be determined by quantifying the reduction of fluorescence as a result of competition. Methods for biotinylating the antigen-binding molecules or antibodies such as antibodies using Sulfo-NHS-biotin or the like are known. Appropriate methods for adding the GST tag to an Fc gamma receptor include methods that involve fusing polypeptides encoding Fc gamma receptor and GST in-frame, expressing the fused gene using cells introduced with a vector carrying the gene, and then purifying using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto the gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the BIACORE™ methods. Examples of such inhibition assay are described in Proc. Natl. Acad. Sci. USA (2006) 103(11), 4005-4010.

### Production and Purification of Multispecific Antigen-Binding Molecules

In some embodiments, multispecific antigen-binding molecules are isolated multispecific antigen-binding molecules.

Multispecific antigen-binding molecules described herein comprise two different antigen-binding moieties (e.g. the “first antigen-binding moiety” and the “second antigen-binding moiety”), fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of multispecific antigen-binding molecules in recombinant production, it will thus be advantageous to introduce in the Fc domain of the multispecific antigen-binding molecule a modification promoting the association of the desired polypeptides.

Accordingly, in particular embodiments, the Fc domain of the multispecific antigen-binding molecule described herein comprises a modification promoting the association of the first and the second subunit of the Fc domain. The site of most extensive protein-protein interaction between the two subunits of a human IgG Fc domain is in the CH3 domain of the Fc domain. Thus, in one embodiment, said modification is in the CH3 domain of the Fc domain.

In a specific embodiment, said modification is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the multispecific antigen-binding molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A).

In yet a further embodiment, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter, J Immunol Methods 248, 7-15 (2001)).

In other embodiments, other techniques for promoting the association among H chains and between L and H chains having the desired combinations can be applied to the multispecific antigen-binding molecules of the present disclosure.

For example, techniques for suppressing undesired H-chain association by introducing electrostatic repulsion at the interface of the second constant region or the third constant region of the antibody H chain (CH2 or CH3) can be applied to multispecific antibody association (WO2006/106905).

In the technique of suppressing unintended H-chain association by introducing electrostatic repulsion at the interface of CH2 or CH3, examples of amino acid residues in contact at the interface of the other constant region of the H chain include regions corresponding to the residues at EU numbering positions 356, 439, 357, 370, 399, and 409 in the CH3 region.

More specifically, examples include an antibody comprising two types of H-chain CH3 regions, in which one to three pairs of amino acid residues in the first H-chain CH3 region, selected from the pairs of amino acid residues indicated in (1) to (3) below, carry the same type of charge: (1) amino acid residues comprised in the H chain CH3 region at EU numbering positions 356 and 439; (2) amino acid residues comprised in the H-chain CH3 region at EU numbering positions 357 and 370; and (3) amino acid residues comprised in the H-chain CH3 region at EU numbering positions 399 and 409.

Furthermore, the antibody may be an antibody in which pairs of the amino acid residues in the second H-chain CH3 region which is different from the first H-chain CH3 region mentioned above, are selected from the aforementioned pairs of amino acid residues of (1) to (3), wherein the one to three pairs of amino acid residues that correspond to the aforementioned pairs of amino acid residues of (1) to (3) carrying the same type of charges in the first H-chain CH3 region mentioned above carry opposite charges from the corresponding amino acid residues in the first H-chain CH3 region mentioned above.

Each of the amino acid residues indicated in (1) to (3) above come close to each other during association. Those skilled in the art can find out positions that correspond to the above-mentioned amino acid residues of (1) to (3) in a desired H-chain CH3 region or H-chain constant region by homology modeling and such using commercially available software, and amino acid residues of these positions can be appropriately subjected to modification.

In the antibodies mentioned above, “charged amino acid residues” are preferably selected, for example, from amino acid residues included in either one of the following groups:

(a) glutamic acid (E) and aspartic acid (D); and
(b) lysine (K), arginine (R), and histidine (H).

In the above-mentioned antibodies, the phrase “carrying the same charge” means, for example, that all of the two or more amino acid residues are selected from the amino acid residues included in either one of groups (a) and (b) mentioned above. The phrase “carrying opposite charges” means, for example, that when at least one of the amino acid residues among two or more amino acid residues is selected from the amino acid residues included in either one of groups (a) and (b) mentioned above, the remaining amino acid residues are selected from the amino acid residues included in the other group.

In a preferred embodiment, the antibodies mentioned above may have their first H-chain CH3 region and second H-chain CH3 region crosslinked by disulfide bonds.

In the present disclosure, amino acid residues subjected to modification are not limited to the above-mentioned amino acid residues of the antibody variable regions or the antibody constant regions. Those skilled in the art can identify the amino acid residues that form an interface in mutant polypeptides or heteromultimers by homology modeling and such using commercially available software; and amino acid residues of these positions can then be subjected to modification so as to regulate the association.

In addition, other known techniques can also be used for formation of multispecific antigen-binding molecules of the present disclosure. Association of polypeptides having different sequences can be induced efficiently by complementary association of CH3 using a strand-exchange engineered domain CH3 produced by changing part of one of the H-chain CH3s of an antibody to a corresponding IgA-derived sequence and introducing a corresponding IgA-derived sequence into the complementary portion of the other H-chain CH3 (Protein Engineering Design & Selection, 23; 195-202, 2010). This known technique can also be used to efficiently form multispecific antigen-binding molecules of interest.

In addition, technologies for antibody production using association of antibody CH1 and CL and association of VH and VL as described in WO 2011/028952, WO2014/018572, and Nat Biotechnol. 2014 February; 32(2):191-8; technologies for producing bispecific antibodies using separately prepared monoclonal antibodies in combination (Fab Arm Exchange) as described in WO2008/119353 and WO2011/131746; technologies for regulating association between antibody heavy-chain CH3s as described in WO2012/058768 and WO2013/063702; technologies for producing multispecific antibodies composed of two types of light chains and one type of heavy chain as described in WO2012/023053; technologies for producing multispecific antibodies using two bacterial cell strains that individually express one of the chains of an antibody comprising a single H chain and a single L chain as described by Christoph et al. (Nature Biotechnology Vol. 31, p 753-758 (2013)); and such may be used for the formation of multispecific antigen-binding molecules.

Alternatively, even when a multispecific antigen-binding molecule of interest cannot be formed efficiently, a multispecific antigen-binding molecule of the present disclosure can be obtained by separating and purifying the multispecific antigen-binding molecule of interest from the produced molecules. For example, a method for enabling purification of two types of homomeric forms and the heteromeric antibody of interest by ion-exchange chromatography by imparting a difference in isoelectric points by introducing amino acid substitutions into the variable regions of the two types of H chains has been reported (WO2007114325). To date, as a method for purifying heteromeric antibodies, methods using Protein A to purify a heterodimeric antibody comprising a mouse IgG2a H chain that binds to Protein A and a rat IgG2b H chain that does not bind to Protein A have been reported (WO98050431 and WO95033844). Furthermore, a heterodimeric antibody can be purified efficiently on its own by using H chains comprising substitution of amino acid residues at EU numbering positions 435 and 436, which is the IgG-Protein A binding site, with Tyr, His, or such which are amino acids that yield a different Protein A affinity, or using H chains with a different protein A affinity, to change the interaction of each of the H chains with Protein A, and then using a Protein A column.

Furthermore, an Fc region whose Fc region C-terminal heterogeneity has been improved can be appropriately used as an Fc region of the present disclosure. More specifically, the present disclosure provides Fc regions produced by deleting glycine at position 446 and lysine at position 447 as specified by EU numbering from the amino acid sequences of two polypeptides constituting an Fc region derived from IgG1, IgG2, IgG3, or IgG4.

Multispecific antigen-binding molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the multispecific antigen-binding molecule binds. For example, for affinity chromatography purification of multispecific antigen-binding molecules of the invention, a matrix with protein A or protein G may be used. Sequential Protein A or G affinity chromatography and size exclusion chromatography can be used to isolate a multispecific antigen-binding molecule. The purity of the multispecific antigen-binding molecule can be determined by any of a variety of well-known analytical methods including gel electrophoresis, high pressure liquid chromatography, and the like.

### Antibody-Dependent Cell-Mediated Cytotoxicity

“Antibody-dependent cell-mediated cytotoxicity” or “ADCC” refers to a form of cytotoxicity in which secreted Ig bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express Fc gamma RIII only, whereas monocytes express Fc gamma RI, Fc gamma RII, and Fc gamma RIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

### Complement Dependent Cytotoxicity

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

### T Cell Dependent Cellular Cytotoxicity

“T cell dependent cellular cytotoxicity” or “TDCC” refers to a form of cytotoxicity in which an antigen-binding molecule binds to both an antigen expressed on the target cell, and another antigen expressed on T cell, that redirect T cell near to the target cell, as cytotoxicity against the target cell is induced due to the T cell. The method to assess T cell dependent cellular cytotoxicity, an in vitro TDCC assay, is also described in the “Measurement of T cell dependent cellular cytotoxicity” section of this description.

### Measurement of T Cell Dependent Cellular Cytotoxicity

In the embodiment that the antigen-binding molecule binds to both CLDN6 and CD3/CD137, the methods described below are preferably used as a method for assessing or determining T cell dependent cellular cytotoxicity (TDCC) caused by contacting an antigen-binding molecule of the present disclosure with CLDN6-expressing cells to which the antigen-binding site in the antigen-binding molecules of the present disclosure binds. The methods for assessing or determining the cytotoxic activity in vitro include methods for determining the activity of cytotoxic T-cells or the like. Whether an antigen-binding molecule of the present disclosure has the activity of inducing T-cell mediated cellular cytotoxicity can be determined by known methods (see, for example, Current protocols in Immunology, Chapter 7. Immunologic studies in humans, Editor, John E, Coligan et al., John Wiley & Sons, Inc., (1993)). In the cytotoxicity assay, an antigen-binding molecule which is able to bind to an antigen different from CLDN6 and which is not expressed in the cells, and CD3/CD137, is used as a control antigen-binding molecule. The control antigen-binding molecule is assayed in the same manner. Then, the activity is assessed by testing whether an antigen-binding molecule of the present disclosure exhibits a stronger cytotoxic activity than that of a control antigen-binding molecule.

Meanwhile, the in vivo anti-tumor efficacy is assessed or determined, for example, by the following procedure. Cells expressing the antigen to which the antigen-binding site in an antigen-binding molecule of the present disclosure binds are transplanted intracutaneously or subcutaneously to a nonhuman animal subject. Then, from the day of transplantation or thereafter, a test antigen-binding molecule is administered into vein or peritoneal cavity every day or at intervals of several days. The tumor size is measured over time. Difference in the change of tumor size can be defined as the cytotoxic activity. As in an in vitro assay, a control antigen-binding molecule is administered. The antigen-binding molecule of the present disclosure can be judged to have cytotoxic activity when the tumor size is smaller in the group administered with the antigen-binding molecule of the present disclosure than in the group administered with the control antigen-binding molecule.

An MTT method and measurement of isotope-labeled thymidine uptake into cells are preferably used to assess or determine the effect of contact with an antigen-binding molecule of the present disclosure to suppress the growth of cells expressing an antigen to which the antigen-binding site in the antigen-binding molecule binds. Meanwhile, the same methods described above for assessing or determining the in vivo cytotoxic activity can be used preferably to assess or determine the activity of suppressing cell growth in vivo.

The TDCC of an antibody or antigen-binding molecule of the disclosure can be evaluated by any suitable method known in the art. For example, TDCC can be measured by lactate dehydrogenase (LDH) release assay. In this assay, target cells (e.g. CLDN6-expressing cells) are incubated with T cells (e.g. PBMCs) in the presence of a test antibody or antigen-binding molecule, and the activity of LDH that has been released from target cells killed by T cells is measured using a suitable reagent. Typically, the cytotoxic activity is calculated as a percentage of the LDH activity resulting from the incubation with the antibody or antigen-binding molecule relative to the LDH activity resulting from 100% killing of target cells (e.g. lysed by treatment with Triton-X). If the cytotoxic activity calculated as mentioned above is higher, the test antibody or antigen-binding molecule is determined to have higher TDCC.

Additionally or alternatively, for example, TDCC can also be measured by real-time cell growth inhibition assay. In this assay, target cells (e.g. CLDN6-expressing cells) are incubated with T cells (e.g. PBMCs) in the presence of a test antibody or antigen-binding molecule on a 96-well plate, and the growth of the target cells is monitored by methods known in the art, for example, by using a suitable analyzing instrument (e.g. xCELLigence Real-Time Cell Analyzer). The rate of cell growth inhibition (CGI: %) is determined from the cell index value according to the formulation given as CGI (%)=100-(CIAb×100/CINoAb). “CIAb” represents the cell index value of wells with the antibody or antigen-binding molecule on a specific experimental time and “CINoAb” represents the average cell index value of wells without the antibody or antigen-binding molecule. If the CGI rate of the antibody or antigen-binding molecule is high, i.e., has a significantly positive value, it can be said that the antibody or antigen-binding molecule has TDCC activity.

In one aspect, an antibody or antigen-binding molecule of the disclosure has T cell activation activity. T cell activation can be assayed by methods known in the art, such as a method using an engineered T cell line that expresses a reporter gene (e.g. luciferase) in response to its activation (e.g. Jurkat/NFAT-RE Reporter Cell Line (T Cell Activation Bioassay, Promega)). In this method, target cells (e.g. CLDN6-expressing cells) are cultured with T cells in the presence of a test antibody or antigen-binding molecule, and then the level or activity of the expression product of the reporter gene is measured by appropriate methods as an index of T cell activation. When the reporter gene is a luciferase gene, luminescence arising from reaction between luciferase and its substrate may be measured as an index of T cell activation. If T cell activation measured as described above is higher, the test antibody or antigen-binding molecule is determined to have higher T cell activation activity.

### Pharmaceutical Composition

In one aspect, the present disclosure provides a pharmaceutical composition comprising the antigen-binding molecule or antibody of the disclosure. In certain embodiments, the pharmaceutical composition of the disclosure induces T-cell-dependent cytotoxicity, in another word, the pharmaceutical composition of the disclosure is a therapeutic agent for inducing cellular cytotoxicity. In certain embodiments, the pharmaceutical composition of the disclosure is a pharmaceutical composition used for treatment and/or prevention of cancer. In certain embodiments, the pharmaceutical composition of the disclosure is a pharmaceutical composition used for treatment and/or prevention of CLDN6-positive cancer or CLDN6-expressing cancer including ovarian cancer, non-small cell lung cancer, gastric cancer, liver cancer, endometrial cancer, or germ cell tumor; and other CLDN6-positive cancer or CLDN6-expressing cancer. In certain embodiments, the pharmaceutical composition of the disclosure is cell growth-suppressing agent. In certain embodiments, the pharmaceutical composition of the disclosure is anticancer agent.

A pharmaceutical composition of the present disclosure, a therapeutic agent for inducing cellular cytotoxicity, a cell growth-suppressing agent, or an anticancer agent of the present disclosure may be formulated with different types of antigen-binding molecules or antibodies, if needed. For example, the cytotoxic action against cells expressing an antigen can be enhanced by a cocktail of multiple antigen-binding molecules or antibodies of the disclosure.

Pharmaceutical compositions comprising an antigen-binding molecule or antibody as described herein are prepared by mixing such antigen-binding molecule or antibody having the desired degree of purity with one or more optional pharmaceutically acceptable carriers (Remington's Pharmaceutical Sciences 16^{th} edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. Such active ingredients are suitably present in combination in amounts that are effective for the purpose intended.

If necessary, the antigen-binding molecules or antibodies of the present disclosure may be encapsulated in microcapsules (microcapsules made from hydroxymethylcellulose, gelatin, poly[methylmethacrylate], and the like), and made into components of colloidal drug delivery systems (liposomes, albumin microspheres, microemulsions, nano-particles, and nano-capsules) (for example, see “Remington's Pharmaceutical Science 16^{th} edition”, Oslo Ed. (1980)). Moreover, methods for preparing agents as sustained-release agents are known, and these can be applied to the antigen-binding molecules of the present disclosure (J. Biomed. Mater. Res. (1981) 15, 267-277; Chemtech. (1982) 12, 98-105; U.S. Pat. No. 3,773,719; European Patent Application (EP) Nos. EP58481 and EP133988; Biopolymers (1983) 22, 547-556).

If necessary, the vectors comprising nucleic acid molecule encodes the antigen-binding molecules or antibodies of the present disclosure may be introduced to subjects, to express the antigen-binding molecules or antibodies of the present disclosure directly within the subject. An example of vectors that is possible to be used is adenovirus, but not limited to. It is also possible to administer the nucleic acid molecule encodes the antigen-binding molecules or antibodies of the present disclosure directly into a subject, or transfer the nucleic acid molecule encodes the antigen-binding molecules or antibodies of the present disclosure via electroporation to a subject, or administer cells comprises nucleic acid molecule encodes the antigen-binding molecules or antibodies of the present disclosure to be expressed and secreted into a subject, to express and secrete the antigen-binding molecules or antibodies of the present disclosure in the subject continuously.

The pharmaceutical compositions, cell growth-suppressing agents, or anticancer agents of the present disclosure may be administered either orally or parenterally to patients. Parental administration is preferred. Specifically, such administration methods include injection, nasal administration, transpulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present disclosure can be administered locally or systemically by injection. Furthermore, appropriate administration methods can be selected according to the patient's age and symptoms. The administered dose can be selected, for example, from the range of 0.0001 mg to 1,000 mg per kg of body weight for each administration. Alternatively, the dose can be selected, for example, from the range of 0.001 mg/body to 100,000 mg/body per patient. However, the dose of a pharmaceutical composition of the present disclosure is not limited to these doses.

Preferably, a pharmaceutical composition of the present disclosure comprises an antigen-binding molecule or antibody as described herein. In one aspect, the composition is a pharmaceutical composition for use in inducing cellular cytotoxicity. In another aspect, the composition is a pharmaceutical composition for use in treating or preventing cancer. Preferably, the cancer is colorectal cancer or gastric cancer. The pharmaceutical composition of the present disclosure can be used for treating or preventing cancer. Thus, the present disclosure provides a method for treating or preventing cancer, in which the antigen-binding molecule or antibody as described herein is administered to a patient in need thereof.

The present disclosure also provides methods for damaging cells expressing CLDN6 or CLDN6-positive cancer, or for suppressing the cell growth by contacting the cells expressing CLDN6 with an antigen-binding molecule of the present disclosure that binds to CLDN6. Cells to which an antigen-binding molecule of the present disclosure binds are not particularly limited, as long as they express CLDN6. Specifically, in the present disclosure, the preferred CLDN6-expressing cancer or CLDN6-positive cancer including ovarian cancer, non-small cell lung cancer, gastric cancer, liver cancer, endometrial cancer, or germ cell tumor.

In the present disclosure, “contact” can be carried out, for example, by adding an antigen-binding molecule of the present disclosure to culture media of cells expressing CLDN6 cultured in vitro. In this case, an antigen-binding molecule to be added can be used in an appropriate form, such as a solution or solid prepared by lyophilization or the like. When the antigen-binding molecule of the present disclosure is added as an aqueous solution, the solution may be a pure aqueous solution containing the antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The added concentration is not particularly limited; however, the final concentration in a culture medium is preferably in a range of 1 pg/ml to 1 g/ml, more preferably 1 ng/ml to 1 mg/ml, and still more preferably 1 micro g/ml to 1 mg/ml.

In another embodiment of the present disclosure, “contact” can also be carried out by administration to nonhuman animals transplanted with CLDN6-expressing cells in vivo or to animals having cancer cells expressing CLDN6 endogenously. The administration method may be oral or parenteral. Parenteral administration is particularly preferred. Specifically, the parenteral administration method includes injection, nasal administration, pulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present disclosure can be administered locally or systemically by injection. Furthermore, an appropriate administration method can be selected according to the age and symptoms of an animal subject. When the antigen-binding molecule is administered as an aqueous solution, the solution may be a pure aqueous solution containing the antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The administered dose can be selected, for example, from the range of 0.0001 to 1,000 mg per kg of body weight for each administration. Alternatively, the dose can be selected, for example, from the range of 0.001 to 100,000 mg/body for each patient. However, the dose of an antigen-binding molecule of the present disclosure is not limited to these examples.

The present disclosure also provides kits for use in a method of the present disclosure, which contain an antigen-binding molecule of the present disclosure or an antigen-binding molecule produced by a method of the present disclosure. The kits may be packaged with an additional pharmaceutically acceptable carrier or medium, or instruction manual describing how to use the kits, etc.

In another aspect of the invention, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Package Insert

The term “package insert” is used to refer to instructions customarily included in commercial packages of therapeutic products, that contain information about the indications, usage, dosage, administration, combination therapy, contraindications and/or warnings concerning the use of such therapeutic products.

### Pharmaceutical Formulation

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

### Pharmaceutically Acceptable Carrier

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

### Treatment

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antigen-binding molecules or antibodies of the present disclosure are used to delay development of a disease or to slow the progression of a disease.

### Cancer

The terms “cancer” and “cancerous” refer to or describe the physiological condition in mammals that is typically characterized by unregulated cell growth/proliferation.

In certain embodiments, the cancer is a CLDN6-expressing or CLDN6-positive cancer which include ovarian cancer, non-small cell lung cancer, gastric cancer, liver cancer, endometrial cancer, or germ cell tumor; and other CLDN6-positive cancer or CLDN6-expressing cancer.

### Tumor

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

### Other Agents and Treatments

The multispecific antigen-binding molecules described herein may be administered in combination with one or more other agents in therapy. For instance, a multispecific antigen-binding molecules as described herein may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of multispecific antigen-binding molecules used, the type of disorder or treatment, and other factors discussed above. The multispecific antigen-binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the multispecific antigen-binding molecules described herein can occur prior to, simultaneously, and/or following, administration of the additional therapeutic agent and/or adjuvant. Multispecific antigen-binding molecules as described herein can also be used in combination with radiation therapy.

All documents cited herein are incorporated herein by reference.

The following are examples of methods and compositions of the present disclosure. It is understood that various other embodiments may be practiced, given the general description provided above.

### EXAMPLES

### Example 1 Screening of Affinity Matured Variants Derived from Parental Dual-Fab H183L072 for Improvement in In Vitro Cytotoxicity on Tumor Cells

### 1.1 Sequence of Affinity Matured Variants

To increase the binding affinity of parental Dual-Fab H183L072 (Heavy chain: SEQ ID NO: 90; Light chain: SEQ ID NO: 142), more than 1,000 Dual-Fab variants were generated using H183L072 as a template by introducing single or multiple mutations on variable region. Antibodies were expressed using Expi293 (Invitrogen) and purified by Protein A purification followed by gel filtration, when gel filtration was necessary. The sequences of 15 represented variants with multiple mutations are listed in Table 1 and binding kinetics were evaluated at 25 degrees C. and/or 37 degrees C. using Biacore T200 instrument (GE Healthcare) as described below in the Example 1.2.2.

TABLE 1

| | | | SEQ ID NOs | | | | | | | |
|---|---|---|---|---|---|---|---|---|---|---|
| variant name | VH name | VL name | VH | H-CDR1 | H-CDR2 | H-CDR3 | VL | L-CDR1 | L-CDR2 | L-CDR3 |
| H183/L072 | dBBDu183H | dBBDu072L | 90 | 103 | 116 | 129 | 142 | 147 | 152 | 157 |
| H0868L0581 | dBBDu183H0868 | dBBDu072L0581 | 91 | 104 | 117 | 130 | 143 | 148 | 153 | 158 |
| H1550L0918 | dBBDu183H1550 | dBBDu072L0918 | 92 | 105 | 118 | 131 | 144 | 149 | 154 | 159 |
| H1571L0581 | dBBDu183H1571 | dBBDu072L0581 | 93 | 106 | 119 | 132 | 143 | 148 | 153 | 158 |
| H1610L0581 | dBBDu183H1610 | dBBDu072L0581 | 94 | 107 | 120 | 133 | 143 | 148 | 153 | 158 |
| H1610L0939 | dBBDu183H1610 | dBBDu072L0939 | 94 | 107 | 120 | 133 | 145 | 150 | 155 | 160 |
| H1643L0581 | dBBDu183H1643 | dBBDu072L0581 | 95 | 108 | 121 | 134 | 143 | 148 | 153 | 158 |
| H1647L0581 | dBBDu183H1647 | dBBDu072L0581 | 96 | 109 | 122 | 135 | 143 | 148 | 153 | 158 |
| H1649L0581 | dBBDu183H1649 | dBBDu072L0581 | 97 | 110 | 123 | 136 | 143 | 148 | 153 | 158 |
| H1649L0943 | dBBDu183H1649 | dBBDu072L0943 | 97 | 110 | 123 | 136 | 146 | 151 | 156 | 161 |
| H1651L0581 | dBBDu183H1651 | dBBDu072L0581 | 98 | 111 | 124 | 137 | 143 | 148 | 153 | 158 |
| H1652L0943 | dBBDu183H1652 | dBBDu072L0943 | 99 | 112 | 125 | 138 | 146 | 151 | 156 | 161 |
| H1673L0943 | dBBDu183H1673 | dBBDu072L0943 | 100 | 113 | 126 | 139 | 146 | 151 | 156 | 161 |
| H1673L0581 | dBBDu183H1673 | dBBDu072L0581 | 100 | 113 | 126 | 139 | 143 | 148 | 153 | 158 |
| H2591L0581 | dBBDu183H2591 | dBBDu072L0581 | 101 | 114 | 127 | 140 | 143 | 148 | 153 | 158 |
| H2594L0581 | dBBDu183H2594 | dBBDu072L0581 | 102 | 115 | 128 | 141 | 143 | 148 | 153 | 158 |
| CD3ε | CD3εVH | CD3εVL | 162 | | | | 163 | | | |
| CD137 | CD137VH | CD137VL | 164 | | | | 165 | | | |

### 1.2. Binding Kinetics Information of Affinity Matured Variants

### 1.2.1 Expression and Purification of Human CD3 and CD137

The gamma and epsilon subunits of the human CD3 complex (human CD3eg linker) were linked by a 29-mer linker and a Flag-tag was fused to the C-terminal end of the gamma subunit (SEQ ID NO: 169). This construct was expressed transiently using FreeStyle293F cell line (Thermo Fisher). Conditioned media expressing human CD3eg linker was concentrated using a column packed with Q HP resins (GE healthcare) and then applied to FLAG-tag affinity chromatography. Fractions containing human CD3eg linker were collected and subsequently subjected to a Superdex 200 gel filtration column (GE healthcare) equilibrated with 1x D-PBS. Fractions containing human CD3eg linker were then pooled and stored at −80 degrees C.

Human CD137 extracellular domain (ECD) (SEQ ID NO: 179) with hexahistidine (His-tag) and biotin acceptor peptide (BAP) on its C-terminus was expressed transiently using FreeStyle293F cell line (Thermo Fisher). Conditioned media expressing human CD137 ECD was applied to a HisTrap HP column (GE healthcare) and eluted with buffer containing imidazole (Nacalai). Fractions containing human CD137 ECD were collected and subsequently subjected to a Superdex 200 gel filtration column (GE healthcare) equilibrated with 1x D-PB S. Fractions containing human CD137 ECD were then pooled and stored at −80 degrees C.

### 1.2.2 Affinity Measurement Towards Human CD3 and CD137

Binding affinity of Dual-Fab antibodies (Dual-Ig) to human CD3 were assessed at 25 degrees C. using Biacore T200 instrument (GE Healthcare). Anti-human Fc (GE Healthcare) was immobilized onto all flow cells of a CM4 sensor chip using amine coupling kit (GE Healthcare). Antibodies were captured onto the anti-Fc sensor surfaces, then recombinant human CD3 or CD137 was injected over the flow cell. All antibodies and analytes were prepared in ACES pH 7.4 containing 20 mM ACES, 150 mM NaCl, 0.05% Tween 20, and 0.005% NaN3. Sensor surface was regenerated each cycle with 3M MgCl₂. Binding affinity were determined by processing and fitting the data to 1:1 binding model using Biacore T200 Evaluation software, version 2.0 (GE Healthcare). CD137 binding affinity assay was conducted in same condition except assay temperature was set at 37 degrees C. Binding affinity of Dual-Fab antibodies to recombinant human CD3 and CD137 are shown in Table 2 (the expression E used to express the Kₒₙ, K_{off}, and KD values in the table means “10 to the power of” and, for instance, 3.54E+04=3.54*10⁴).

TABLE 2

| | CD3 | | | CD137 | | |
|---|---|---|---|---|---|---|
| | Kon | Koff | | Kon | Koff | |
| | (M − 1 · s − 1) | (s−1) | KD (M) | (M − 1 · s − 1) | (s − 1) | KD (M) |
| H183L072 | 3.54E+04 | 1.20E−02 | 3.40E−07 | 3.47E+03 | 1.96E−02 | 5.66E−06 |
| H0868L0581 | 1.23E+05 | 1.94E−02 | 1.57E−07 | 1.22E+04 | 1.36E−03 | 1.11E−07 |
| H1550L0918 | 7.20E+04 | 3.16E−03 | 4.38E−08 | 1.09E+04 | 5.79E−03 | 5.30E−07 |
| H1571L0581 | 1.42E+05 | 1.56E−02 | 1.10E−07 | 1.21E+04 | 1.05E−03 | 8.68E−08 |
| H1610L0581 | 6.80E+04 | 1.42E−03 | 2.09E−08 | 1.07E+04 | 1.10E−03 | 1.03E−07 |
| H1610L0939 | 5.00E+04 | 2.53E−03 | 5.07E−08 | 1.30E+04 | 8.01E−04 | 6.18E−08 |
| H1643L0581 | 9.46E+04 | 2.51E−02 | 2.65E−07 | 1.23E+04 | 6.06E−04 | 4.94E−08 |
| H1647L0581 | 4.43E+04 | 1.01E−01 | 2.28E−06 | 9.98E+03 | 6.47E−04 | 6.48E−08 |
| H1649L0581 | 7.50E+04 | 3.36E−02 | 4.49E−07 | 1.29E+04 | 5.53E−04 | 4.28E−08 |
| H1649L0943 | 6.10E+04 | 4.81E−02 | 7.89E−07 | 1.43E+04 | 4.68E−04 | 3.28E−08 |
| H1651L0581 | 7.18E+04 | 3.71E−02 | 5.17E−07 | 1.40E+04 | 6.03E−04 | 4.32E−08 |
| H1652L0943 | 6.23E+04 | 6.36E−02 | 1.02E−06 | 1.29E+04 | 4.70E−04 | 3.64E−08 |
| H1673L0581 | 7.96E+04 | 1.06E−03 | 1.33E−08 | 1.19E+04 | 9.60E−04 | 8.04E−08 |
| H1673L0943 | 5.50E+04 | 1.16E−03 | 2.10E−08 | 1.22E+04 | 7.22E−04 | 5.91E−08 |
| H2591L0581 | 1.02E+05 | 5.35E−02 | 5.25E−07 | 2.04E+04 | 7.42E−04 | 3.63E−08 |
| H2594L0581 | 9.83E+04 | 5.84E−02 | 5.93E−07 | 2.09E+04 | 1.63E−03 | 7.81E−08 |

### Example 2 X-Ray Crystal Structure Analysis of H0868L0581/hCD137 Complex

### 2.1. Preparation of Antibody for Co-Crystal Analysis

H0868L581 was selected for co-crystal analysis with hCD137 protein. The bivalent antibody was transiently transfected and expressed using an Expi293 Expression system (Thermo Fisher Scientific). Culture supernatants were harvested and antibodies were purified from the supernatants using MabSelect SuRe affinity chromatography (GE Healthcare) followed by gel filtration chromatography using Superdex200 (GE Healthcare).

### 2.2. Expression and Purification of Extracellular Domain (24-186) of Human CD137

Extracellular domain of human CD137 fused to Fc via Factor Xa cleavable linker (CD137-FFc, SEQ ID NO: 166) was expressed in the HEK293 Cell in the presence of kifunensine. The CD137-FFc from culture medium was purified by affinity chromatography (HiTrap MabSelect SuRe column, GE Healthcare) and size exclusion chromatography (HiLoad 16/600 Superdex 200 pg column, GE healthcare). Fc was cleaved with Factor Xa and the resultant CD137 extracellular domain was further purified with tandemly connected gel filtration column (HiLoad 16/600 Superdex 200 pg, GE healthcare) and Protein A column (HiTrap MabSelect SuRe 1 ml, GE Healthcare) and subsequently purified using Benzamidine Sepharose resin (GE Healthcare). Fractions containing CD137 extracellular domain were pooled and stored at −80 degrees C.

### 2.3. Preparation of Fab Fragment of H0868L0581 and Anti-CD137 Control Antibody

Antibodies for crystal structure analysis were transiently transfected and expressed using an Expi293 Expression system (Thermo Fisher Scientific). Culture supernatants were harvested and antibodies were purified from the supernatants using MabSelect SuRe affinity chromatography (GE Healthcare) followed by gel filtration chromatography using Superdex200 (GE Healthcare). Fab fragments of H0868L0581 and known anti-CD137 control antibody (called as 137Ctrl hereafter, Heavy chain SEQ ID NO: 167, Light chain SEQ ID NO: 168) were prepared by the conventional method using limited digestion with Lys-C(Roche), followed by loading onto a protein A column (MabSlect SuRe, GE Healthcare) to remove Fc fragments, a cation exchange column (HiTrap SP HP, GE Healthcare), and a gel filtration column (Superdex200 16/60, GE Healthcare). Fractions containing Fab fragment were pooled and stored at −80 degrees C.

### 2.4. Preparation of H0868L0581 Fab, 137Ctrl and Human CD137 Complex

Purified CD137 was mixed with GST-tag fused Endoglycosidase F1 (in-house) for deglycosylation, followed by purification of CD137 using gel filtration column (HiLoad 16/600 Superdex 200 pg, GE healthcare) and Protein A column (HiTrap MabSelect SuRe 1 ml, GE Healthcare). Purified CD137 was mixed with H0868L0581 Fab. The complex was purified by gel filtration column (Superdex 200 Increase 10/300 GL, GE healthcare) and subsequently purified H0868L0581 Fab and CD137 complex was mixed with 137Ctrl. The ternary complex was purified by gel filtration chromatography (Superdex200 10/300 increase, GE Healthcare) using a column equilibrated with 25 mM HEPES pH 7.3, 100 mM NaCl.

### 2.5. Crystallization

The purified complexes were concentrated to about 10 mg/mL, and crystallization was carried out by the sitting drop vapor diffusion method at 21 degrees C. The reservoir solution consisted of 0.1M Tris hydrochloride pH8.5, 25.0% v/v Polyethylene glycol monomethyl ether 550.

### 2.6. Data Collection and Structure Determination

X-ray diffraction data were measured by X06SA at SLS. During the measurement, the crystal was constantly placed in a nitrogen stream at −178 degrees C. to maintain it in a frozen state, and a total of 1440 X-ray diffraction images were collected using an Eiger X16M (DECTRIS) attached to a beam line, while rotating the crystal 0.25 degrees at a time. Determining the cell parameters, indexing the diffraction spots, and processing the diffraction data obtained from the diffraction images were performed using the autoPROC program (Acta. Cryst. 2011, D67: 293-302), XDS Package (Acta. Cryst. 2010, D66: 125-132), and AIMLESS (Acta. Cryst. 2013, D69: 1204-1214), and finally the diffraction intensity data up to 3.705 angstrom resolution was obtained. The crystallography data statistics are shown in Table 3.

The structure was determined by molecular replacement with the program Phaser (J. Appl. Cryst. 2007, 40: 658-674). The search model was derived from the published crystal structure (PDB code: 4NKI and 6MI2). A model was built with the Coot program (Acta Cryst. 2010, D66: 486-501) and refined with the program Refmac5 (Acta Cryst. 2011, D67: 355-367) and PHENIX (Acta Cryst. 2010, D66: 213-221). The crystallographic reliability factor (R) for the diffraction intensity data from 77.585-3.705 angstrom was 22.33%, with a Free R value of 27.50%. The structure refinement statistics are shown in Table 3.

TABLE 3
X-ray data collection and refinement statistics

| Data collection | |
|---|---|
| Space group | C2 |
| Unit Cell | |
| a, b, c (Å) | 233.795, 74.019, 81.986 |
| α, β, γ (°) | 90.000, 108.858, 90.000 |
| Resolution (Å) | 77.585-3.705 |
| Total reflections | 99,488 |
| Unique reflections | 14,221 |
| Completeness (highest resolution shell) (%) | 99.2 (99.7) |
| R_{merge} ^{a} (highest resolution shell) | 0.161 (1.052) |
| Refinement | |
| Resolution (Å) | 48.822-3.705 |
| Reflections | 14,195 |
| R factor ^{b} (R_{free} ^{c}) (%) | 22.33 (27.50) |
| rms deviation from ideal | |
| Bond lengths (Å) | 0.003 |
| Bond angles (°) | 0.618 |

^{a} R_{merge} = ΣhklΣj|Ij (hkl) − <I (hkl)> |/ΣhklΣj|Ij (hkl)|, where Ij (hkl) and <I (hkl)> are the intensity of measurement j and the mean intensity for the reflection with indices hkl, respectively.
^{b} R factor = Σhkl|F_{calc}(hkl)| − |F_{obs} (hkl)|/Σhkl|F_{obs} (hkl)|, where F_{obs} and F_{calc} are the observed and calculated structure factor amplitudes, respectively.
^{c} R_{free} is calculated with 5% of the reflection randomly set aside.

### 2.7. Identification of the Interaction Sites of H0868L0581 Fab and CD137

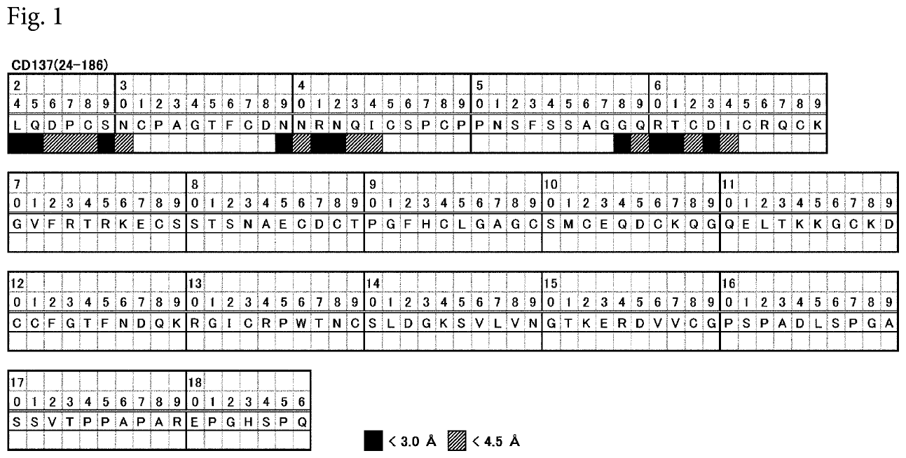

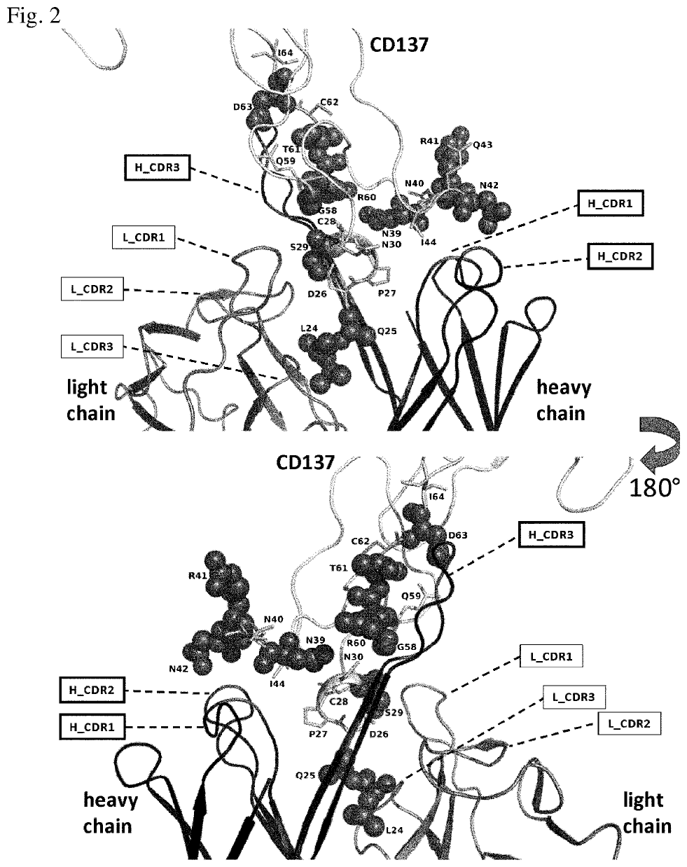

The crystal structure of the ternary complex of H0868L0581 Fab, 137Ctrl and CD137 was determined at 3.705 angstrom resolution. In FIGS. 1 and 2, the epitope of the H0868L0581 Fab contact region is mapped in the CD137 amino acid sequence and in the crystal structure, respectively. The epitope includes the amino acid residues of CD137 that contain one or more atoms located within 4.5 angstrom distance from any part of the H0868L0581 Fab in the crystal structure. In addition, the epitope within 3.0 angstrom is highlighted in FIGS. 1 and 2.

As shown in FIGS. 1 and 2, the crystal structure showed that the L24-N30 in CRD1 of CD137 bound in a pocket formed between Heavy chain and Light chain of H0868L0581 Fab, particularly L24-S29 were deeply buried in a manner that the N-terminus of CD137 was oriented toward the depth of the pocket. In addition, N39-I44 in CRD1 and G58-I64 in CRD2 in CD137 were recognized by Heavy chain CDRs of H0868L0581 Fab. CRD is the name of domains divided by the structure formed by Cys-Cys called CRD reference as described in WO2015/156268.

We identified an anti-human CD137 antibody which recognizes the N-terminus region, especially L24-N30, of human CD137, and also identified that the antibody against this region can activate CD137 on cells.

### Example 3 Generation of Anti-CLDN6/Dual-Fab Tri-Specific Antibody

Tri-specific antibodies with one arm targeting claudin-6 and the other arm with dual targeting function to CD3 and CD137 were generated by utilizing FAST-Ig (WO2013065708) or CrossMab technology (FIG. 3). The target antigen of each Fv region in the tri-specific antibodies was shown in Table 4. The naming rule of each of chain was shown in FIG. 3, and the SEQ ID NOs are shown in Table 5. Sequence of each variable region is shown in Table 6.

Fc region was Fc gamma R silent and deglycosylated. FcRn enhanced mutations Act5 (M428L, N434A, Q438R, S440E) were applied to improve PK of antibodies. The engineered components applied to each antibody were shown in Tables 7-1 and 7-2, with the details of FAST06, FAST22 and FAST30 shown in Table 8. All antibodies were expressed as tri-specific form by transient expression in Expi293 cells (Invitrogen) and purified according to Reference Example 1.

TABLE 4
Antibody names and Fv names

| Antibody | | Antibody | Fv A | Fv B |
|---|---|---|---|---|
| ID | Antibody name | format | (anti-CLDN6) | (Dual Fab) |
| Ab01 | CLDN6AE25EK/DualAE05KE-SG13251326 | Fast-Ig | CLDN6AE25EK | DualAE05KE |
| Ab02 | CLDN6AE25EK/DualAE15KE-SG13251326 | Fast-Ig | CLDN6AE25EK | DualAE15KE |
| Ab03 | CLDN6AE25/DualAE05-SG14001326 | Fast-Ig | CLDN6AE25 | DualAE05 |
| Ab04 | CLDN6AE25/DualAE15-SG14001326 | Fast-Ig | CLDN6AE25 | DualAE15 |
| Ab05 | xCLDN6AE25/DualAE05-xSG1386k1385hV11 | CrossMab | xCLDN6AE25 | DualAE05 |
| Ab06 | xCLDN6AE25/DualAE15-xSG1386k1385hV11 | CrossMab | xCLDN6AE25 | DualAE15 |
| Ab07 | CLDN6AE25EK/DualAE05KE-SG14051406 | Fast-Ig | CLDN6AE25EK | DualAE05KE |
| Ab08 | CLDN6AE25EK/DualAE15KE-SG14051406 | Fast-Ig | CLDN6AE25EK | DualAE15KE |
| Ab09 | CLDN6AE25/DualAE05-SG14071406 | Fast-Ig | CLDN6AE25 | DualAE05 |
| Ab10 | CLDN6AE25/DualAE15-SG14071406 | Fast-Ig | CLDN6AE25 | DualAE15 |
| Ab11 | xCLDN6AE25/DualAE05-xSG1384k1383hV11 | CrossMab | xCLDN6AE25 | DualAE05 |
| Ab12 | xCLDN6AE25/DualAE15-xSG1384k1383hV11 | CrossMab | xCLDN6AE25 | DualAE15 |
| Ab13 | CLDN6AE25/DualAE05-SG13251326 | Fast-Ig | CLDN6AE25 | DualAE05 |
| Ab14 | CLDN6AE25/DualAE15-SG13251326 | Fast-Ig | CLDN6AE25 | DualAE15 |
| Ab15 | CLDN6AE25/DualAE05-SG14051406 | Fast-Ig | CLDN6AE25 | DualAE05 |
| Ab16 | CLDN6AE25/DualAE15-SG14051406 | Fast-Ig | CLDN6AE25 | DualAE15 |
| Ab17 | xCLDN6AE25/DualAE05-xSG1350k1349hV11 | CrossMab | xCLDN6AE25 | DualAE05 |
| Ab18 | xCLDN6AE25/DualAE15-xSG1350k1349hV11 | CrossMab | xCLDN6AE25 | DualAE15 |

TABLE 5
Antibody names and SEQ ID Nos

| Antibody | | SEQ ID NOs | | | |
|---|---|---|---|---|---|
| ID | Antibody name | Chain 1 | Chain 2 | Chain 3 | Chain 4 |
| Ab01 | CLDN6AE25EK/DualAE05KE-SG13251326 | 41 | 50 | 54 | 68 |
| Ab02 | CLDN6AE25EK/DualAE15KE-SG13251326 | 41 | 50 | 55 | 68 |
| Ab03 | CLDN6AE25/DualAE05-SG14001326 | 42 | 51 | 56 | 69 |
| Ab04 | CLDN6AE25/DualAE15-SG14001326 | 42 | 51 | 57 | 69 |
| Ab05 | xCLDN6AE25/DualAE05-xSG1386k1385hV11 | 44 | 52 | 60 | 70 |
| Ab06 | xCLDN6AE25/DualAE15-xSG1386k1385hV11 | 44 | 52 | 61 | 70 |
| Ab07 | CLDN6AE25EK/DualAE05KE-SG14051406 | 45 | 50 | 62 | 68 |
| Ab08 | CLDN6AE25EK/DualAE15KE-SG14051406 | 45 | 50 | 63 | 68 |
| Ab09 | CLDN6AE25/DualAE05-SG14071406 | 46 | 51 | 64 | 69 |
| Ab10 | CLDN6AE25/DualAE15-SG14071406 | 46 | 51 | 65 | 69 |
| Ab11 | xCLDN6AE25/DualAE05-xSG1384k1383hV11 | 47 | 52 | 66 | 70 |
| Ab12 | xCLDN6AE25/DualAE15-xSG1384k1383hV11 | 47 | 52 | 67 | 70 |
| Ab13 | CLDN6AE25/DualAE05-SG13251326 | 48 | 53 | 56 | 71 |
| Ab14 | CLDN6AE25/DualAE15-SG13251326 | 48 | 53 | 57 | 71 |
| Ab15 | CLDN6AE25/DualAE05-SG14051406 | 49 | 53 | 64 | 71 |
| Ab16 | CLDN6AE25/DualAE15-SG14051406 | 49 | 53 | 65 | 71 |
| Ab17 | xCLDN6AE25/DualAE05-xSG1350k1349hV11 | 43 | 52 | 58 | 70 |
| Ab18 | xCLDN6AE25/DualAE15-xSG1350k1349hV11 | 43 | 52 | 59 | 70 |

TABLE 6
Fv names, VH, VL and their CDR1-3 SEQ ID NOs

| | | | SEQ ID NOs | | | | | | | |
|---|---|---|---|---|---|---|---|---|---|---|
| | | | Variable region in the same | | | | Variable region in the same | | | |
| | Variable region in the | Variable region in the | polypeptide chain with heavy | | | | polypeptide chain with light | | | |
| | same polypeptide chain | same polypeptide chain | chain constant region | | | | chain constant region | | | |
| | with heavy chain | with light chain | Variable | | | | Variable | | | |
| Fv name | constant region | constant region | region | CDR1 | CDR2 | CDR3 | region | CDR1 | CDR2 | CDR3 |
| CLDN6AE25EK | 65HQ39E | 54L0532Q38K | 2 | 8 | 14 | 20 | 26 | 30 | 34 | 38 |
| CLDN6AE25 | 65H | 54L0532 | 1 | 7 | 13 | 19 | 25 | 29 | 33 | 37 |
| xCLDN6AE25 | 54L0532 | 65H | 25 | 29 | 33 | 37 | 1 | 7 | 13 | 19 |
| DualAE05KE | dBBDu183H1643•Q39K | dBBDu072L0581•Q38E | 3 | 9 | 15 | 21 | 27 | 31 | 35 | 39 |
| DualAE15KE | dBBDu183H2594•Q39K | dBBDu072L0581•Q38E | 4 | 10 | 16 | 22 | 27 | 31 | 35 | 39 |
| DualAE05 | dBBDu183H1643 | dBBDu072L0581 | 5 | 11 | 17 | 23 | 28 | 32 | 36 | 40 |
| DualAE15 | dBBDu183H2594 | dBBDu072L0581 | 6 | 12 | 18 | 24 | 28 | 32 | 36 | 40 |

TABLE 7
Engineered components applied to each antibody

| Antibody | | FcRn binding | H/L | Heavy Chain |
|---|---|---|---|---|
| ID | Antibody name | enhancement | pairing | Paring |
| Ab01 | CLDN6AE25EK/DualAE05KE-SG13251326 | | FAST22 | S3 |
| Ab02 | CLDN6AE25EK/DualAE15KE-SG13251326 | | FAST22 | S3 |
| Ab03 | CLDN6AE25/DualAE05-SG14001326 | | FAST6 | S3 |
| Ab04 | CLDN6AE25/DualAE15-SG14001326 | | FAST6 | S3 |
| Ab05 | xCLDN6AE25/DualAE05-xSG1386k1385hV11 | | Cross-mab | Knob into Hole |
| Ab06 | xCLDN6AE25/DualAE15-xSG1386k1385hV11 | | Cross-mab | Knob into Hole |
| Ab07 | CLDN6AE25EK/DualAE05KE-SG14051406 | Act5 (+) | FAST22 | S3 |
| Ab08 | CLDN6AE25EK/DualAE15KE-SG14051406 | Act5 (+) | FAST22 | S3 |
| Ab09 | CLDN6AE25/DualAE05-SG14071406 | Act5 (+) | FAST6 | S3 |
| Ab10 | CLDN6AE25/DualAE15-SG14071406 | Act5 (+) | FAST6 | S3 |
| Ab11 | xCLDN6AE25/DualAE05-xSG1384k1383hV11 | Act5 (+) | Cross-mab | Knob into Hole |
| Ab12 | xCLDN6AE25/DualAE15-xSG1384k1383hV11 | Act5 (+) | Cross-mab | Knob into Hole |
| Ab13 | CLDN6AE25/DualAE05-SG13251326 | | FAST30 | S3 |
| Ab14 | CLDN6AE25/DualAE15-SG13251326 | | FAST30 | S3 |
| Ab15 | CLDN6AE25/DualAE05-SG14051406 | Act5 (+) | FAST30 | S3 |
| Ab16 | CLDN6AE25/DualAE15-SG14051406 | Act5 (+) | FAST30 | S3 |
| Ab17 | xCLDN6AE25/DualAE05-xSG1350k1349hV11 | | Cross-mab | Knob into Hole |
| Ab18 | xCLDN6AE25/DualAE15-xSG1350k1349hV11 | | Cross-mab | Knob into Hole |

TABLE 8
Details of Fast-Ig engineering in variable regions

| | Dual Fab side (chain 3 and chain 4) | | | | | | | | | anti-CLDN6 side (chain 1 and chain 2) | | | | | | | | |
|---|---|---|---|---|---|---|---|---|---|---|---|---|---|---|---|---|---|---|
| | Heavy chain (chain 3) | | | | Light chain (chain 4) | | | | | Heavy chain (chain 1) | | | | Light chain (chain 2) | | | | |
| No. | Q39 | K147 | Q175 | K213 | Q38 | E123 | S131 | Q160 | T180 | Q39 | K147 | Q175 | K213 | Q38 | E123 | S131 | Q160 | T180 |
| FAST06 | — | — | K | — | — | — | E | E | | | E | E | | | | K | K | |
| FAST22 | K | — | K | — | E | — | E | E | E | E | E | E | E | K | K | K | K | |
| FAST30 | | — | K | — | | — | E | E | E | | E | E | E | | K | K | K | |

### Example 4 FACS Analysis of Specificity Against CLDN Family

Amino acid sequences are highly conserved among CLDN3, CLDN4, CLDN6 and CLDN9. Thus we examined CLDN6 binding specificity of CLDN6 binding Fv comprising 65HQ39E as VH and 54L0532Q38K as VL, by FACS analysis. hCLDN6/BaF, hCLDN3/BaF, hCLDN4/BaF, and hCLDN9/BaF were incubated with an anti-CLDN6/CD3 bispecific antibody (CS2961) comprising the CLDN6 binding Fv CLDN6AE25EK and a CD3 binding Fv (heavy chain variable region SEQ ID NO: 184, light chain variable region SEQ ID NO: 185) at 15 micro g/ml. Another anti-CLDN6/CD3 bispecific antibody (6PHU3/TR01) and an antibody without binding capability to CLDN6 (KLH/TR01) were used as a staining control. 6PHU3/TR01 and KLH/TR01 comprise the same CD3 binding Fv (heavy chain variable region SEQ ID NO: 188, light chain variable region SEQ ID NO: 189). The CLDN6 binding Fv of 6PHU3/TR01 comprises a heavy chain variable region shown in SEQ ID NO: 190 and a light chain variable region shown in SEQ ID NO: 191. KLH/TR01 comprises a KLH binding Fv (heavy chain variable region SEQ ID NO: 186, light chain variable region SEQ ID NO: 187).

Binding of each antibody was detected with Alexa Fluor 488-conjugated anti human IgG (Invitrogen). Dead cells were separated by eFlour 780 (Invitrogen) staining.

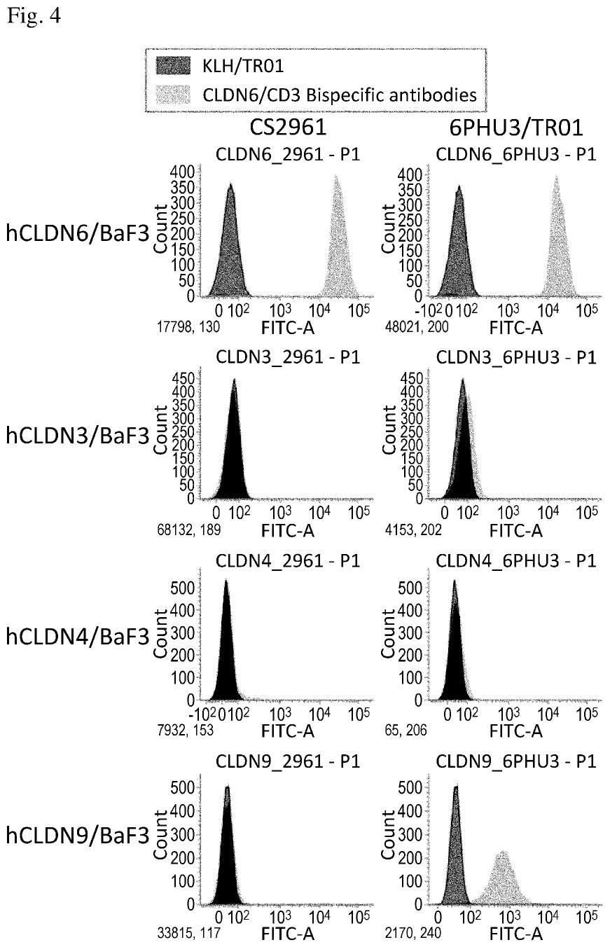

As shown in FIG. 4, CS2961 showed better specificity towards CLDN6 compared with 6PHU3/TR01.

### Example 5 Measurement of T-Cell-Dependent Cell Cytotoxicity of Anti-CLDN6/CD3 Bispecific Antibodies and Anti-CLDN6/Dual-Fab Tri-Specific Antibodies

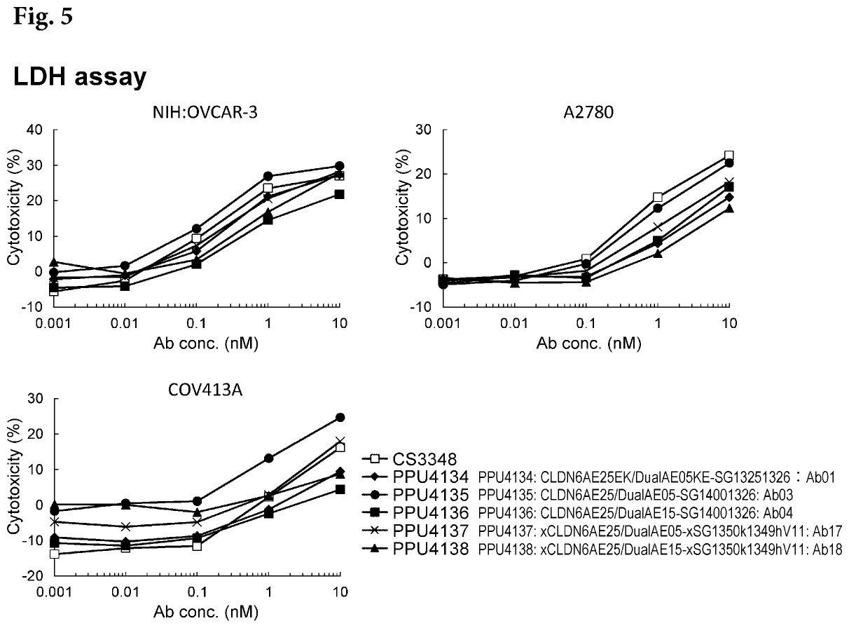

FIG. 5 shows the T-cell-dependent cell cytotoxicity of an anti-CLDN6/CD3 bispecific antibody (CS3348) and five anti-CLDN6/Dual-Fab tri-specific antibodies (PPU4134, PPU4135, PPU4136, PPU4137, and PPU4138) against NIH:OVCAR-3 (high CLDN6 expression ovarian cancer cell line), and A2780 and COV413A (low CLDN6 expression ovarian cancer cell lines). Sequences of the antibodies are shown in Table 9.

TABLE 9

| | | |
|---|---|---|
| Antibody | Other | SEQ ID NOs: |

| name | name | Chain 1 | Chain 2 | Chain 3 | Chain 4 |
|---|---|---|---|---|---|
| CS3348 | — | 193 | 195 | 194 | 192 |
| PPU4134 | Ab01 | 41 | 50 | 54 | 68 |
| PPU4135 | Ab03 | 42 | 51 | 56 | 69 |
| PPU4136 | Ab04 | 42 | 51 | 57 | 69 |
| PPU4137 | Ab17 | 43 | 52 | 58 | 70 |
| PPU4138 | Ab18 | 43 | 52 | 59 | 70 |

Cell cytotoxicity was evaluated by LDH assay using human PBMCs. 15,000 target cells and 150,000 human PBMCs (E/T=10) were seeded into each well of a 96-well U-bottom plate and incubated with various concentrations of antibody for over-night at 37 degree C. and 5% CO₂. Target cell killing was measured by LDH cytotoxicity detection kit (Takara Bio). The cytotoxic activity (%) of each antibody was calculated using the following formula.

Cytotoxic activity(%)=(A−B−C)×100/(D−C)

“A” represents the average absorbance value of wells treated with antibody and PBMCs, “B” represents the average absorbance value of wells with effector cell PBMCs only, “C” represents the average absorbance value of wells with untreated target cells only, and “D” represents the average absorbance value of wells with target cells lysed with Triton-X. Further, the cytotoxicity calculated in a well containing PBMCs and target cells without antibody was set to 0%. All the anti-CLDN6/Dual-Fab tri-specific antibodies showed T-cell-dependent cell cytotoxicity against CLDN6 expressing cells.

### Example 6 Generation of CD137/CD3 Double Humanized Mouse

Human CD137 knock-in (KI) mouse strain was generated by replacing mouse endogenous Cd137 genomic region with human CD137 genomic sequence using mouse embryonic stem cells. Human CD3 EDG-replaced mouse was established as a strain in which all three components of the CD3 complex—CD3e, CD3d, and CD3g—are replaced with their human counterparts, CD3E, CD3D, and CD3G (Scientific Rep. 2018; 8: 46960). CD137/CD3 double humanized mouse strain was established by crossbreeding the human CD137 KI mice with the human CD3 EDG-replaced mice.

### Example 7 Assessment of In Vivo Efficacy of Anti-CLDN6/Dual-Fab Tri-Specific Antibodies with hCD3/hCD137 Mice

Antibodies prepared in Example 3 are evaluated for their in vivo efficacy using tumor-bearing models.

For in vivo efficacy evaluation, CD3/CD137 double humanized mice established in Example 6, which is called as “hCD3/hCD137 mice” hereafter, are used. Cells which have stable expression of human CLDN6 are transplanted into the hCD3/hCD137 mice, and the hCD3/hCD137 mice with confirmed tumor formation are treated by administration of the antibodies.

More specifically, in drug efficacy tests of the antibodies using tumor-bearing models, the tests below are performed. CLDN6 expressing cells (1×10⁶ cells) are transplanted into the inguinal subcutaneous region of hCD3/hCD137 mice. The day of transplantation is defined as day 0. On day 9 after the transplantation, the mice are randomized into groups according to their body weight and tumor size. On the day of randomization, the antibodies are administered intravenously through the caudate vein at 6 mg/kg. The antibodies are administered only once. Tumor volume and body weight are measured with anti-tumor testing system (ANTES version 7.0.0.0) every 3-4 days.

In another in vivo efficacy evaluation, CLDN6 expressing cells are transplanted into the right flank of hCD3/hCD137 mice. On day 9, the mice are randomized into groups on the basis of their tumor volume and body weight, and injected i.v. with vehicle or antibodies prepared in Example 3. Tumor volume is measured twice per week. For IL-6 analysis, mice are bled at 2h after treatment. Plasma samples are analyzed with Bio-Plex Pro Mouse Cytokine Th1 Panel according to the manufacture's protocol.

### Example 8 In Vitro Assay of Cytotoxic Activity Using Lactate Dehydrogenase Release Assay

Cytotoxic activity of the anti-CLDN6/Dual-Fab tri-specific antibody PPU4135 was evaluated by lactate dehydrogenase (LDH) release assay.

Human gastric cancer cell line NUGC-3 (JCRB), human teratocarcinoma cell line PA-1 (ATCC), human uterus cancer cell line SNG-M (JCRB), human testicular germ cell tumor cell line NEC8 (JCRB), and human yolk-sac tumor cell line NEC14 (JCRB), which express human CLDN6, were used as target cells.

After frozen PBMCs (CTL) were washed by CTL anti-aggregate wash and RPMI-1640 Medium (SIGMA) containing 10% FBS (called as 10% FBS/RPMI), the PBMCs were adjusted to 3×10⁶ cells/mL. These PBMCs were used as effector cells.

Target cells were detached from culture flask and seeded at 100 micro L/well containing 1.5×10⁴ cells on u-bottom clear 96-wells plate (Corning). 50 micro L of human PMBC solution (1.5×10⁵ cells) and 50 micro L of the prepared antibody at a concentration selected from 0.004, 0.04, 0.4, 4, or 40 nM were added into wells respectively. After overnight incubation at 37 degree C., plate was centrifuged and 100 micro L culture supernatant from each well was transferred to a new flat bottom clear 96-wells plate. Then 100 micro L of LDH detection reagent (Dye solution containing catalyst, TaKaRa) was added to each well, followed by 30 minutes incubation at room temperature. Absorbance at 490 nm and 620 nm was measured by EnVision (PerkinElmer Japan).

The rate of cytotoxic activity (%) was calculated from the difference between 490 nm and 620 nm absorbance according to following formula.

Cytotoxic activity(%)=(A−B−C)×100/(D−C)

“A” represents the average absorbance value of wells treated with antibody and PBMCs, “B” represents the average absorbance value of wells with effector cell PBMCs only, “C” represents the average absorbance value of wells with untreated target cells only, and “D” represents the average absorbance value of wells with target cells lysed with Triton-X. The average absorbance value of culture medium wells was subtracted from all the absorbance values. Further, the cytotoxicity calculated in a well containing PBMCs and target cells without antibody was set to 0%. The anti-CLDN6/Dual-Fab tri-specific antibodies showed T-cell-dependent cell cytotoxicity against all the cell lines used.

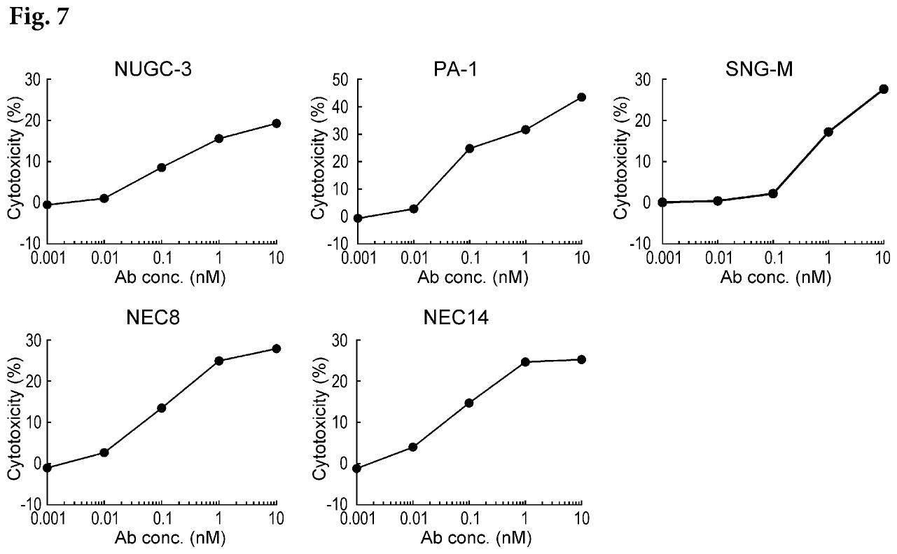

The results are shown in FIG. 7.

### Example 9 Real-Time Cell Growth Inhibition Assay (xCELLigence Assay)

The T-cell dependent growth inhibition mediated by the anti-CLDN6/Dual-Fab tri-specific antibodies was assessed by cell proliferation assay using an xCELLigence RTCA MP instrument (ACEA Biosciences).

Human ovarian cancer cell line NIH:OVCAR-3 (ATCC) and human lung cancer cell line NCI-H1435 (ATCC), which express human CLDN6, were used as target cells.

50 mL of peripheral blood was collected from healthy adult volunteers by a syringe that had been previously injected with 500 micro L of 1,000 units/mL heparin solution (NovoNordisk). Peripheral blood equally divided into four equal parts by dilution with PBS (−) was injected with 15 mL of Ficoll-Paque PLUS and centrifuged in a Leucosep lymphocyte separation tube (Greiner Bio-One). After centrifuging the separation tube (2150 rpm for 10 minutes at room temperature), the peripheral blood mononuclear cell (hereinafter referred as PBMC) fraction layer was separated. After washing the PBMCs once with RPMI-1640 Medium (SIGMA) containing 10% FBS (called as 10% FBS/RPMI), the PBMCs were adjusted to 4×10⁵ cells/mL. These PBMCs were used as effector cells.

1×10⁴ target cells were plated on E-Plate 96 plate (Roche Diagnostics) at 100 micro L/well. After overnight culture, 2×10⁴ T cells together with the antibody at a concentration selected from 0.004, 0.04, 0.4, 4 or 40 nM were added at 50 micro L/well, respectively. Cell growth was monitored every 15 min, using xCELLigence, for 72 hours during the incubation of the plates. The rate of cell growth inhibition (CGI: %) was determined from the cell index value according to the formulation given as CGI (%)=100−(CI_{Ab}×100/CI_{NoAb}) “CIAb” represents the cell index value of wells with antibody on a specific experimental time and “CI_{NoAb}” represents the average cell index value of the wells without antibody at the same experimental time.

The results show that all the anti-CLDN6/Dual-Fab tri-specific antibodies inhibited the cell growth of CLDN6 expressing cancer cell lines (OVCAR-3 and NCI-H1435) in a dose dependent manner

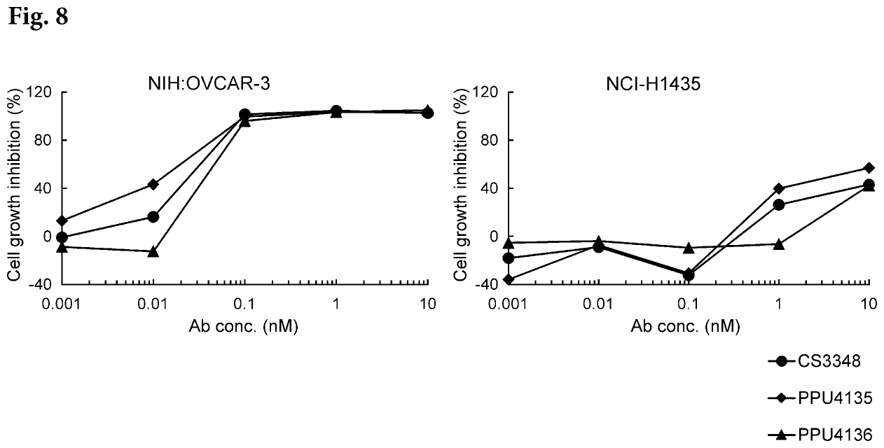

The results are shown in FIG. 8.

### Example 10 T Cell Activation in NFAT-Luc2 Jurkat Cell Lines Co-Cultured with CLDN6 Expressing Tumor Cells

T cell activation through CD3 binding by the anti-CLDN6/Dual-Fab tri-specific antibodies was measured by luciferase assay system using GloResponse NFAT-luc2 Jurkat cells (Promega, J1601) as effector cells. Human ovarian cancer cell line OVCAR-3(ATCC) and lung adenocarcinoma cell lines NCI-H1435 (ATCC) were used as a claudin-6 endogenously expressing cells. Human bladder cancer cell line 5637 (ATCC) was used as CLDN6 negative cells.

Assay was performed as below. First, the above-described cancer cell lines were detached from culture flask and plated at 25 micro L/well (2×10⁴ cells) into white flat bottom 96-wells plate (Coster #3917). Next, 1×10⁵ Jurkat/NFAT-RE Reporter Cell Line together with the antibody at a concentration selected from 0.003, 0.03, 0.3, 3 or 30 nM were added at 25 micro L/well, respectively. After overnight culture at 37 degree C., Bio-Glo reagent (Promega #G7941) was added at 75 micro L/well followed by further incubation at room temperature for 10 minutes. Then, luminescence arising from activating Jurkat cells was measured by EnSpire (PerkinElmer Japan). The luminescence fold of each well was calculated by making comparison between the wells with and without antibody.

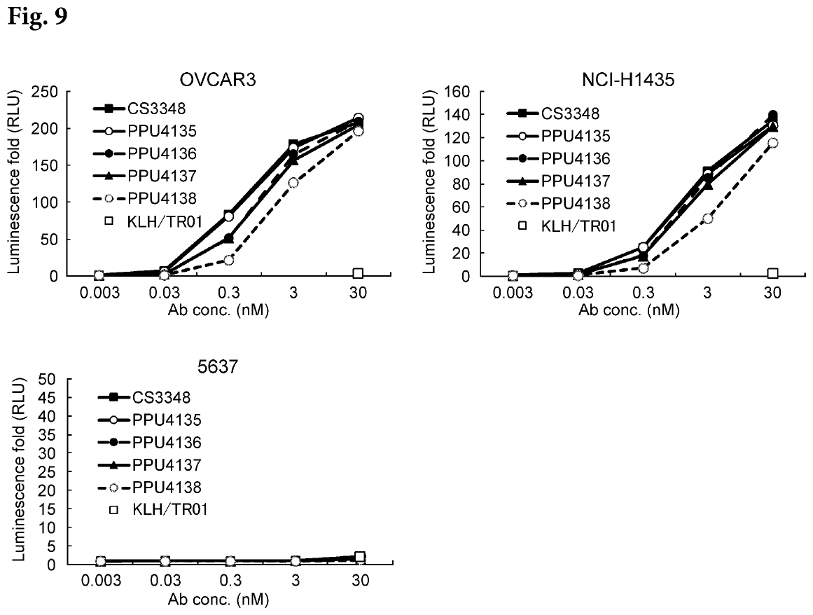

The results of NFAT signal activation properties of anti-CLDN6/Dual-Fab tri-specific antibodies and CS3348 using CLDN6 expressing human cell lines (OVCAR3 and NCI-H1435) and CLDN6 negative cell line (5637) as target cells are shown in FIG. 9.

In the presence of the claudin-6 positive cell lines, NFAT activation by all antibodies was observed in a dose dependent manner. On the other hand, almost no activation was observed even at the high concentration of antibody in the presence of the claudin-6 negative cell line 5637.

### Example 11 NF Kappa B Activation in Jurkat Expressing Human 4-1BB and Luciferase Reporter Cell Lines Co-Cultured with CLDN6 Expressing Tumor Cells

NF kappa B activation through CD137 binding by the anti-CLDN6/Dual-Fab tri-specific antibodies was evaluated using the GloResponse™ NF kappa B luc2/4-1BB Jurkat (Promega, CS196004). Human ovarian cancer cell line OVCAR-3(ATCC) and lung adenocarcinoma cell line NCI-H1435 (ATCC) were used as a claudin-6 endogenously expressing cells. Human bladder cancer cell line 5637 (ATCC) was used as CLDN6 negative cells.

Assay was performed as below. First, the above-described cancer cell lines were detached from culture flask and plated at 25 micro L/well (2.5×10⁴ cells) into white flat bottom 96-wells plate (Coster #3917). Next, 5×10⁴ cells of NF kappa B luc2/4-1BB Jurkat Reporter Cell Line were transferred, and 25 ul medium containing titrated antibodies were mixed. Assay plates were incubated at 37 degree C. for 6 hrs, then Bio-Glo reagent (Promega #G7941) was added at 75 micro L/well followed by further incubation at room temperature for 10 minutes. Then, luminescence arising from activating Jurkat cells was measured by EnVision (PerkinElmer Japan). The luminescence fold of each well was calculated by making comparison between the wells with each antibody (0.003, 0.03, 0.3, 3 and 30 nM) and without antibody.

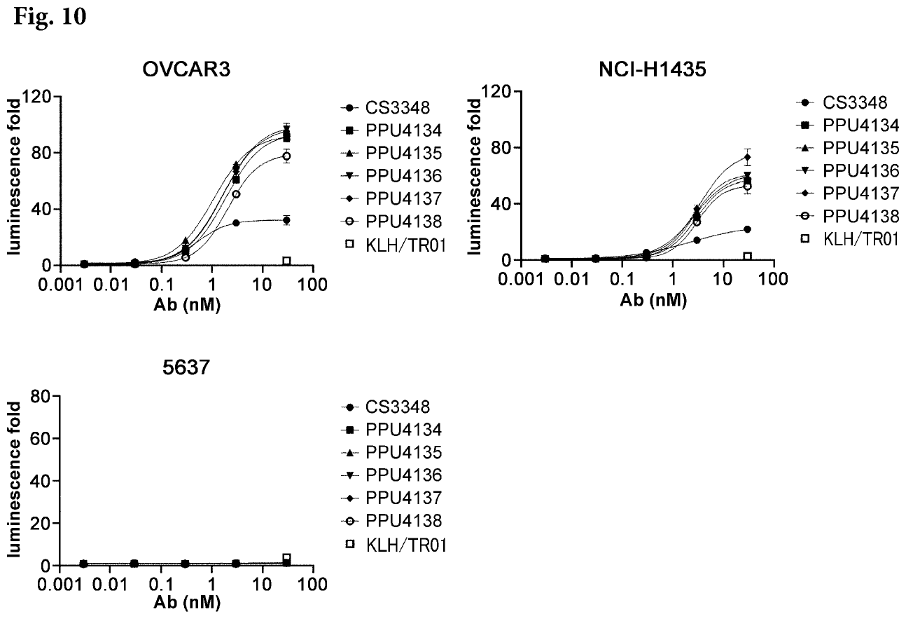

The results of NF kappa B signal activation properties of anti-CLDN6/Dual-Fab tri-specific antibodies and CS3348 using CLDN6 expressing human cell lines (OVCAR3 and NCI-H1435) and CLDN6 negative cell line (5637) as target cells are shown in FIG. 10.

In the presence of the claudin-6 positive cell lines, NF kappa B activation by all antibodies was observed in a dose dependent manner Especially, much greater activation was observed in the presence of anti-CLDN6/Dual-Fab tri-specific antibodies. On the other hand, no activation was observed even at the high concentration of antibody in the presence of the claudin-6 negative cell line 5637.

### Example 12 In Vivo Anti-Tumor Efficacy Study

The in vivo anti-tumor efficacy of the anti-CLDN6/Dual-Fab tri-specific antibodies was evaluated using tumor bearing mice model. The human cancer cell line expressing human CLDN6 (NCI-H1435 or OV-90) was transplanted subcutaneously into humanized NOG mice injected by human stem cell derived from cord blood (HuNOG mice model). Tumor bearing mice were randomized to treatment groups to receive an administration of the antibody, or vehicle as a control (Table 10).

After randomization and grouping the mice according to tumor size and body weight from day 8 (NCI-H1435) or day 16 (0V90) after transplantation, anti-CLDN6/Dual-Fab tri-specific antibodies were administered intravenously. The anti-CLDN6/Dual-Fab tri-specific antibodies were administered only once. The length (L) and width (W) of the tumor mass were measured, and tumor volume (TV) was calculated as: TV=(L×W×W)/2.

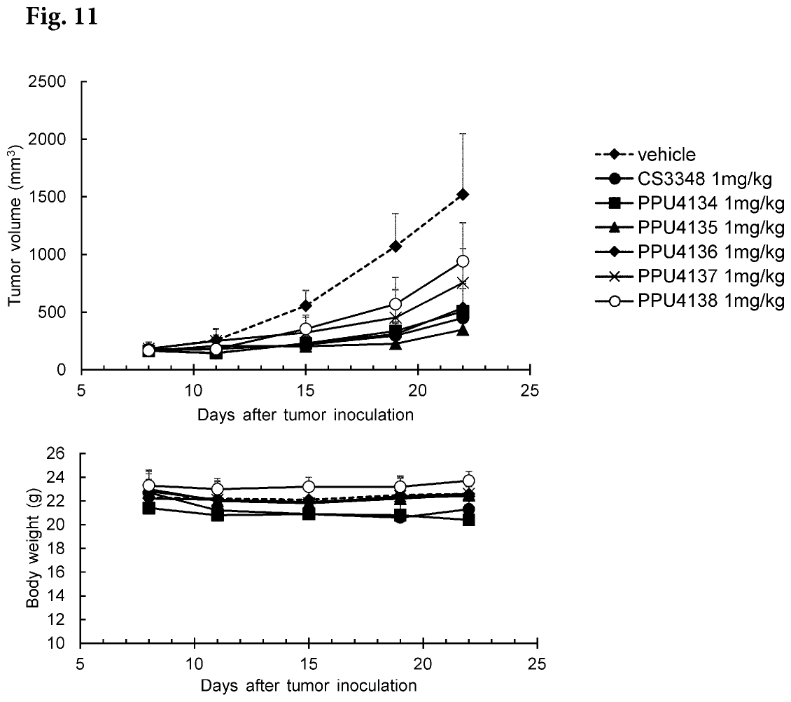

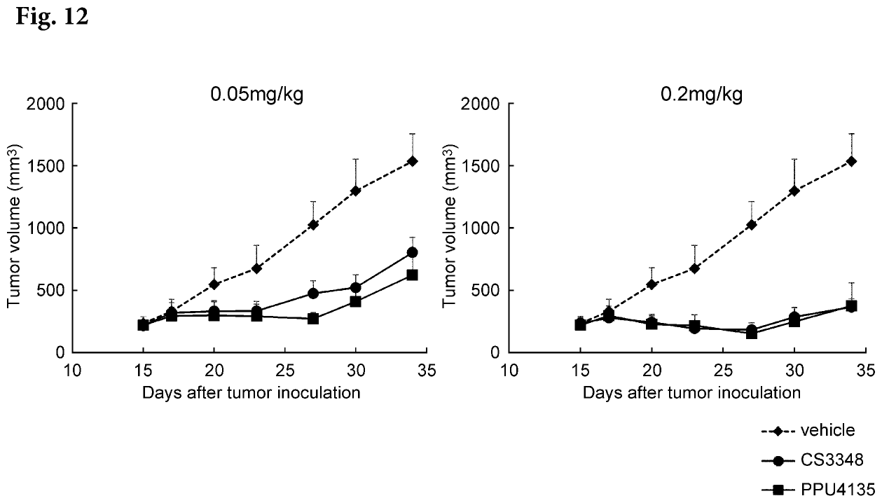

Anti-tumor efficacy was observed in the anti-CLDN6/Dual-Fab tri-specific antibodies-administered groups compared with the vehicle-administered control group (FIGS. 11 and 12).

TABLE 10
Details of study groups for in vivo anti-tumor efficacy evaluation

| | | |
|---|---|---|
| Group | Antibody | Dose |

a. Study groups using NCI-H1435/HuNOG mice model

| 1 | vehicle | — | |
|---|---|---|---|
| 2 | CS3348 | 1 | mg/kg |
| 3 | PPU4134 | 1 | mg/kg |
| 4 | PPU4135 | 1 | mg/kg |
| 5 | PPU4136 | 1 | mg/kg |
| 6 | PPU4137 | 1 | mg/kg |
| 7 | PPU4138 | 1 | mg/kg |

b. Study groups using OV-90/HuNOG mice model

| 1 | vehicle | — | |
|---|---|---|---|
| 2 | CS3348 | 0.05 | mg/kg |
| 3 | CS3348 | 0.2 | mg/kg |
| 4 | PPU4135 | 0.05 | mg/kg |
| 5 | PPU4135 | 0.2 | mg/kg |

### Example 13 Toxicology Study of the Anti-CLDN6/Dual-Fab Tri-Specific Antibody

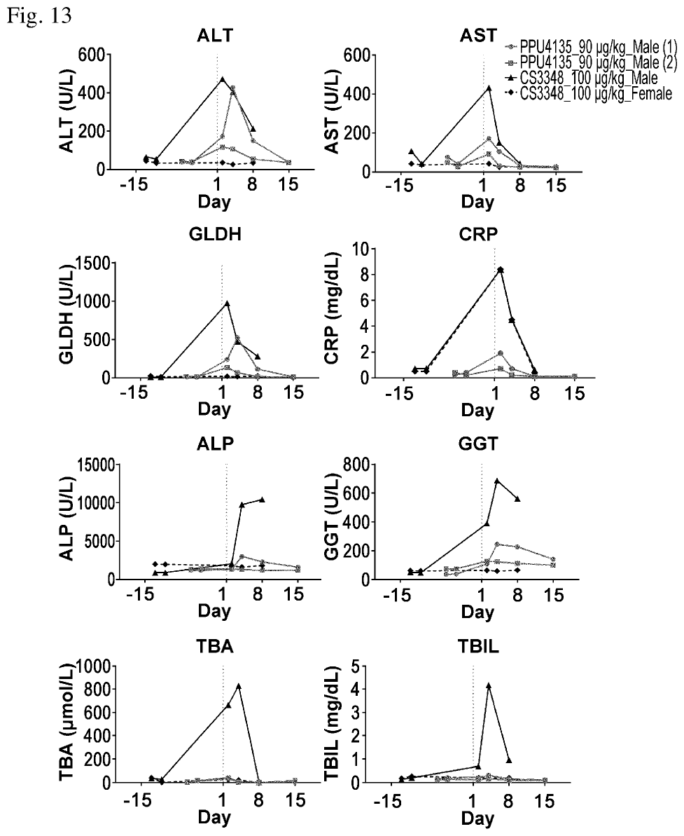

Potential toxicity of PPU4135 antibody (anti-CLDN6/Dual-Fab tri-specific antibody) was evaluated in toxicity study using cynomolgus monkeys compared with CS3348 antibody (anti-CLDN6/CD3 bispecific antibody). Because the both PPU4135 and CS3348 antibodies cross-reacted with their antigens of cynomolgus monkey, cynomolgus monkey was selected as the animal species for evaluations in the in vivo toxicology studies Summary of single dose toxicology studies is shown in Table 11. Since toxicological findings seemed to be more sensitive in male than female animals in the toxicity study with CS3348 (FIG. 13), 2 males were used for evaluation of the PPU4135-mediated toxicity. In these studies, dose levels were set at 100 (for CS3348) or 90 (for PPU4135) micro g/kg, which were approximately 2.57-fold efficacious concentration producing 80% of the maximal response.

TABLE 11
Summary of Toxicology Studies

| | | | | |
|---|---|---|---|---|
| | | Treatment/ | | Dose |
| | | Observation | | (micro |
| Study Type | Species | period | Animals | g/kg) |

Single-dose toxicity:

| | | | | |
|---|---|---|---|---|
| [CS3348] | Cynomolgus | Single IV | 1 male and | 100 |
| Single dose | monkey | dose, 8 days | 1 female | |
| | (3 to 4 years | | | |
| | of age) | | | |
| [PPU4135] | Cynomolgus | Single IV | 2 males | 90 |
| Single dose | monkey | dose, 29 days | | |
| | (4 years of age) | | | |

IV = intravenous

In the male animals treated with CS3348 or PPU4135, plasma exposure levels were comparable between PPU4135 treated group and CS3348 treated group until Day 8. Increased AST (aspartate aminotransferase), ALT (alanine aminotransferase) and GLDH (glutamate dehydrogenase) (hepatic enzymes); ALP (alkaline phosphatase), TBIL (total bilirubin), GGT (gamma glytamyltranspeptidase) and TBA (total bile acid) (hepatobiliary damage parameters); and CRP (C-reactive protein) (inflammatory marker) were noted after single administration of these antibodies (FIG. 13). Although the differences of the hepatic enzyme levels between these antibodies treated male animals were slight (FIG. 13), the hepatobiliary damage parameters and inflammatory marker elevations were dramatically mitigated by PPU4135 administration rather than CS3348 administration throughout the studies (FIG. 13). These results suggest that test article-mediated hepatotoxicity, mainly hepatobiliary damage, is attenuated by using the anti-CLDN6/Dual-Fab tri-specific antibody rather than using the anti-CLDN6/CD3 bispecific antibody.

### Example 14 Characterization of Anti-CLDN6/Dual-Fab Tri-Specific Antibody

The binding affinity of the anti-CLDN6/Dual-Fab tri-specific antibody against human and cynomolgus (cyno) CLDN6 VLP (virus-like particle) at pH 7.4 was determined at 25 degree C. using Biacore T200 instrument (GE Healthcare). Anti-human CD81 (BD Pharmingen) antibody was immobilized onto all flow cells of a C1 sensor chip using amine coupling kit (GE Healthcare). Human and cyno CLDN6 VLP were captured onto the sensor surface by the anti-human CD81 antibody. Each VLP was 5-fold dilution by buffer (20 mM NaPhosphate, 150 mM NaCl, 0.1 mg/mL BSA, 0.005% NaN₃, pH 7.4). Tested antibody was prepared in buffer (20 mM NaPhosphate, 150 mM NaCl, 0.1 mg/mL BSA, 0.005% NaN₃, pH 7.4). Anti-CLDN6/Dual-Fab tri-specific antibody was injected at 50 and 200 nM, followed by dissociation. Sensor surface was regenerated each cycle with 0.1% SDS and 100 mM H₃PO₄. As shown in Table 12, binding affinity of PPU4135 towards cyno CLDN6 is comparable with that towards human CLDN6. Binding affinities were determined by processing and fitting the data to 1:1 binding model using Biacore T200 Evaluation software, version 2.0 (GE Healthcare).

The binding affinity of the anti-CLDN6/Dual-Fab tri-specific antibody against recombinant human and cyno CD3eg (gamma and epsilon subunits of CD3) at pH 7.4 was determined at 25 degree C. using Biacore 8K instrument (GE Healthcare). The binding affinity of the anti-CLDN6/Dual-Fab tri-specific antibody against recombinant human and cyno CD137 at pH 7.4 was determined at 37 degree C. using Biacore 8K instrument (GE Healthcare). Anti-human Fc (GE Healthcare) antibody was immobilized onto all flow cells of a CM4 sensor chip using amine coupling kit (GE Healthcare). Tested antibody and analytes were prepared in ACES pH 7.4 containing 20 mM ACES, 150 mM NaCl, 0.05% Tween 20, 0.005% NaN3. Anti-CLDN6/Dual-Fab tri-specific antibody was captured onto the sensor surface by anti-human Fc. Antibody capture levels were aimed at 300 resonance unit (RU). Recombinant CD3eg and CD137 were injected at both 500 and 2000 nM, followed by dissociation. Sensor surface was regenerated each cycle with 3M MgCl₂. As shown in Table 12, binding affinities of PPU4135 towards cyno CD3eg and cyno CD137 are comparable with those towards human CD3eg and CD137, respectively. Binding affinities were determined by processing and fitting the data to 1:1 binding model using Biacore Insight Evaluation software (GE Healthcare).

TABLE 12
Binding affinity of PPU4135 towards human and cyno antigens

| | affinity | | |
|---|---|---|---|
| Antigen | ka (1/Ms) | kd (1/s) | KD (M) |
| human CLDN6 | 2.17E+05 | 7.94E−04 | 3.65E−09 |
| cyno CLDN6 | 2.18E+05 | 8.21E−04 | 3.77E−09 |
| human CD3eg | 7.80E+04 | 2.66E−02 | 3.41E−07 |
| cyno CD3eg | 1.01E+05 | 3.32E−02 | 3.29E−07 |
| human CD137 | 1.26E+04 | 5.91E−04 | 4.68E−08 |
| cyno CD137 | 8.35E+03 | 1.46E−03 | 1.74E−07 |

(The expression E used to express the ka (1/Ms), kd (1/s), and KD values in the table means “10 to the power of” and, for instance, 2.17E+05=2.17*10⁵)

### Reference Example 1 Purification of Anti-CLDN6/Dual-Fab Tri-Specific Antibodies

The heavy and light chain variable regions were cloned into expression vectors containing the heavy chain and light chain constant regions with respective mutations for hetero-dimerization.

For large scale preparation of anti-CLDN6/Dual-Fab tri-specific antibodies for in vitro and in vivo studies, the antibodies were transiently expressed using Expi293F cells (Life technologies), according to the manufacturer's instructions. Culture medium containing recombinant antibodies was first purified with MabSelect Sure (GE healthcare) column and eluted with 50 mM Acetic acid. Eluted antibodies were neutralized with 1.5M Tris HCl/1M Arginine HCl buffer. ProA eluates were then loaded onto the cation exchange HiTrap SP-HP (GE healthcare) column in 20 mM Sodium Phosphate, pH6 buffer and eluted with 20 mM Sodium Phosphate, 1M NaCl, pH6 buffer. Fractions containing the bispecific antibody were pooled and concentrated. To remove high molecular weight and/or low molecular weight components, size exclusion chromatography was performed in P1 buffer (20 mM Histidine, 150 mM Arginine, 162.1 mM Asp, pH6.0) using Superdex 200 column (GE healthcare). Purified bispecific antibodies were concentrated and stored in −80 degrees C. freezer.

### Reference Example 2 Generation of Claudin Expressing Cells

Ba/F3 cells expressing human CLDN6 (hCLDN6/BaF), Ba/F3 cells expressing human CLDN9 (hCLDN9/BaF), Ba/F3 cells expressing human CLDN3 (hCLDN3/BaF), Ba/F3 cells expressing human CLDN4 (hCLDN4/BaF), Ba/F3 cells expressing mouse CLDN6 (mCLDN6/BaF), Ba/F3 cells expressing mouse CLDN9 (mCLDN9/BaF), Ba/F3 cells expressing mouse CLDN3 (mCLDN3/BaF), and Ba/F3 cells expressing mouse CLDN4 (mCLDN4/BaF), were established by transfecting human CLDN6, human CLDN9 (SEQ ID NO: 198), human CLDN3 (SEQ ID NO: 199), human CLDN4 (SEQ ID NO: 200), mouse CLDN6 (SEQ ID NO: 201), mouse CLDN9 (SEQ ID NO: 202), mouse CLDN3 (SEQ ID NO: 203), and mouse CLDN4 (SEQ ID NO:204) expression vectors into mouse pro B cell line Ba/F3 respectively.

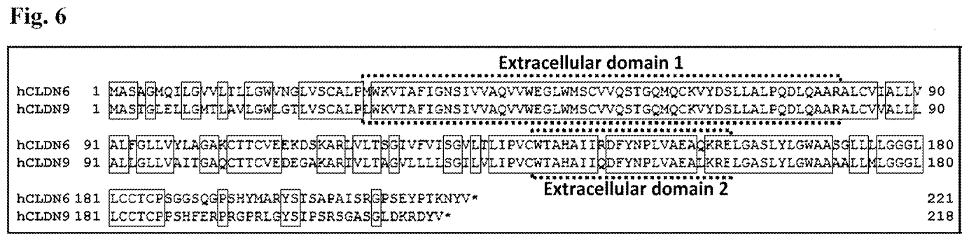

Claudin family proteins have two extracellular domains which are accessible to antibody. With regard to amino acid sequence similarity between the extracellular domains of human CLDN6 and human CLDN9, the first extracellular domain is almost the same, and there are only two different amino acids in the second extracellular domain (FIG. 6). The glutamine at position 156 of human Claudin 6 (position 156 in the sequence shown in SEQ ID NO: 196 or 197) was substituted to leucine to make a human CLDN6 mutant comprising the same amino acid as human Claudin 9 at position 156. This human CLDN6 mutant was named as hCLDN6(Q156L) (SEQ ID NO: 205). Ba/F3 transfectant stably expressing hCLDN6(Q156L) was generated using similar method described above. The established Ba/F3 transfectant was named as hCLDN6(Q156L)/BaF.

FreeStyle™ 293-F transfectant cells transiently expressing human and mouse CLDN3, 4, 6, and 9 were generated by introducing expressing vector of human and mouse CLDNs (including CLDN6, CLDN9, CLDN3, and CLDN4) into FreeStyle™ 293-F cells (Invitrogen) using 293fectin (Invitrogen). The generated FreeStyle™ 293-F transfectant cells were named as hCLDN3/FS293, hCLDN4/FS293, hCLDN6/FS293, hCLDN9/FS293, mCLDN3/FS293, mCLDN4/FS293, mCLDN6/FS293, and mCLDN9/FS293, respectively.

### INDUSTRIAL APPLICABILITY

The present disclosure provides multispecific antigen-binding molecules capable of binding to CD3 and CD137 (4-1BB) but not binding to CD3 and CD137 at the same time, and capable of binding to CLDN6. The multispecific antigen-binding molecules of the present disclosure exhibit enhanced T-cell dependent cytotoxicity activity in a CLDN6-dependent manner through binding to the CD3/CD37 and CLDN6. The multispecific antigen-binding molecules and pharmaceutical compositions thereof can be used for targeting cells expressing CLDN6, for use in immunotherapy for treating various cancers, especially those associated with CLDN6 such as CLDN6-positive cancers.

Sequence Listing C1-A2011Psq. txt

1. A multispecific antigen-binding molecule comprising:
(i) a first antigen-binding moiety that is capable of binding to CD3 and CD137, wherein the first antigen-binding moiety binds to either CD3 or CD137; and
(ii) a second antigen-binding moiety that is capable of binding to claudin-6 (CLDN6).

2. A multispecific antigen-binding molecule comprising:
a first antigen-binding moiety wherein the first antigen-binding moiety has at least one property selected from:
(i) binding to CD3,
(ii) binding to CD137, and
(iii) being capable of binding to CD3 and CD137,
wherein the first antigen-binding moiety binds to either CD3 or CD137; and
a second antigen-binding moiety, wherein the second antigen-binding moiety is capable of binding to claudin-6 (CLDN6) and;
wherein the first antigen-binding moiety comprises:
(a1) a first antibody variable region comprising the complementarity determining region (CDR) 1 of SEQ ID NO: 9, the CDR 2 of SEQ ID NO: 15, and the CDR 3 of SEQ ID NO: 21, and a second antibody variable region comprising the CDR 1 of SEQ ID NO: 31, the CDR 2 of SEQ ID NO: 35, and the CDR 3 of SEQ ID NO: 39;
(a2) a first antibody variable region comprising the complementarity determining region (CDR) 1 of SEQ ID NO: 10, the CDR 2 of SEQ ID NO: 16, and the CDR 3 of SEQ ID NO: 22, and a second antibody variable region comprising the CDR 1 of SEQ ID NO: 31, the CDR 2 of SEQ ID NO: 35, and the CDR 3 of SEQ ID NO: 39;
(a3) a first antibody variable region comprising the complementarity determining region (CDR) 1 of SEQ ID NO: 11, the CDR 2 of SEQ ID NO: 17, and the CDR 3 of SEQ ID NO: 23, and a second antibody variable region comprising the CDR 1 of SEQ ID NO: 32, the CDR 2 of SEQ ID NO: 36, and the CDR 3 of SEQ ID NO: 40; or
(a4) a first antibody variable region comprising the complementarity determining region (CDR) 1 of SEQ ID NO: 12, the CDR 2 of SEQ ID NO: 18, and the CDR 3 of SEQ ID NO: 24, and a second antibody variable region comprising the CDR 1 of SEQ ID NO: 32, the CDR 2 of SEQ ID NO: 36, and the CDR 3 of SEQ ID NO: 40.

3. The multispecific antigen-binding molecule of claim 2, wherein the second antigen-binding moiety comprises:
(b1) a third antibody variable region comprising the complementarity determining region (CDR) 1 of SEQ ID NO: 8, the CDR 2 of SEQ ID NO: 14, and the CDR 3 of SEQ ID NO: 20, and a fourth antibody variable region comprising the CDR 1 of SEQ ID NO: 30, the CDR 2 of SEQ ID NO: 34, and the CDR 3 of SEQ ID NO: 38;
(b2) a third antibody variable region comprising the complementarity determining region (CDR) 1 of SEQ ID NO: 7, the CDR 2 of SEQ ID NO: 13, and the CDR 3 of SEQ ID NO: 19, and a fourth antibody variable region comprising the CDR 1 of SEQ ID NO: 29, the CDR 2 of SEQ ID NO: 33, and the CDR 3 of SEQ ID NO: 37; or
(b3) a third antibody variable region comprising the complementarity determining region (CDR) 1 of SEQ ID NO: 29, the CDR 2 of SEQ ID NO: 33, and the CDR 3 of SEQ ID NO: 37, and a fourth antibody variable region comprising the CDR 1 of SEQ ID NO: 7, the CDR 2 of SEQ ID NO: 13, and the CDR 3 of SEQ ID NO: 19.

4. The multispecific antigen-binding molecule of claim 2, wherein the antibody variable regions comprised in the first and/or second antigen-binding moiety comprise human antibody frameworks or humanized antibody frameworks.

5. A multispecific antigen-binding molecule comprising:
a first antigen-binding moiety wherein the first antigen-binding moiety has at least one property selected from:
(i) binding to CD3,
(ii) binding to CD137, and
(iii) being capable of binding to CD3 and CD137,
wherein the first antigen-binding moiety binds to either CD3 or CD137; and
a second antigen-binding moiety, wherein the second antigen-binding moiety is capable of binding to claudin-6 (CLDN6); and
wherein the first antigen-binding moiety comprises:
(c1) a first antibody variable region comprising the amino acid sequence of SEQ ID NO: 3, and a second antibody variable region comprising the amino acid sequence of SEQ ID NO: 27;
(c2) a first antibody variable region comprising the amino acid sequence of SEQ ID NO: 4, and a second antibody variable region comprising the amino acid sequence of SEQ ID NO: 27;
(c3) a first antibody variable region comprising the amino acid sequence of SEQ ID NO: 5, and a second antibody variable region comprising the amino acid sequence of SEQ ID NO: 28; or,
(c4) a first antibody variable region comprising the amino acid sequence of SEQ ID NO: 6, and a second antibody variable region comprising the amino acid sequence of SEQ ID NO: 28.

6. The multispecific antigen-binding molecule of claim 5, wherein the second antigen-binding moiety comprises:
(d1) a third antibody variable region comprising the amino acid sequence of SEQ ID NO: 2, and a fourth antibody variable region comprising the amino acid sequence of SEQ ID NO: 26;
(d2) a third antibody variable region comprising the amino acid sequence of SEQ ID NO: 1, and a fourth antibody variable region comprising the amino acid sequence of SEQ ID NO: 25; or
(d3) a third antibody variable region comprising the amino acid sequence of SEQ ID NO: 25, and a fourth antibody variable region comprising the amino acid sequence of SEQ ID NO: 1.

7. The multispecific antigen-binding molecule of claim 2, which further comprises:
(iii) an Fc domain which exhibits reduced binding affinity to human Fc gamma receptor, as compared to a native human IgG1 Fc domain.

8. The multispecific antigen-binding molecule of claim 2, wherein the first antibody variable region of the first antigen-binding moiety is fused with a first heavy chain constant region, the second antibody variable region of the first antigen-binding moiety is fused with a first light chain constant region, the third antibody variable region of the second antigen-binding moiety is fused with a second heavy chain constant region, the fourth antibody variable region of the second antigen-binding moiety is fused with a second light chain constant region, wherein the constant regions comprise:
(g1) a first heavy chain constant region comprising the amino acid sequence of SEQ ID NO: 74, a first light chain constant region comprising the amino acid sequence of SEQ ID NO: 87, a second heavy chain constant region comprising the amino acid sequence of SEQ ID NO: 73, and a second light chain constant region comprising the amino acid sequence of SEQ ID NO: 88;
(g2) a first heavy chain constant region comprising the amino acid sequence of SEQ ID NO: 74, a first light chain constant region comprising the amino acid sequence of SEQ ID NO: 85, a second heavy chain constant region comprising the amino acid sequence of SEQ ID NO: 81, and a second light chain constant region comprising the amino acid sequence of SEQ ID NO: 86;
(g3) a first heavy chain constant region comprising the amino acid sequence of SEQ ID NO: 79, a first light chain constant region comprising the amino acid sequence of SEQ ID NO: 72, a second heavy chain constant region comprising the amino acid sequence of SEQ ID NO: 80, and a second light chain constant region comprising the amino acid sequence of SEQ ID NO: 89;
(g4) a first heavy chain constant region comprising the amino acid sequence of SEQ ID NO: 83, a first light chain constant region comprising the amino acid sequence of SEQ ID NO: 87, a second heavy chain constant region comprising the amino acid sequence of SEQ ID NO: 82, and a second light chain constant region comprising the amino acid sequence of SEQ ID NO: 88;
(g5) a first heavy chain constant region comprising the amino acid sequence of SEQ ID NO: 83, a first light chain constant region comprising the amino acid sequence of SEQ ID NO: 85, a second heavy chain constant region comprising the amino acid sequence of SEQ ID NO: 84, and a second light chain constant region comprising the amino acid sequence of SEQ ID NO: 86;
(g6) a first heavy chain constant region comprising the amino acid sequence of SEQ ID NO: 77, a first light chain constant region comprising the amino acid sequence of SEQ ID NO: 72, a second heavy chain constant region comprising the amino acid sequence of SEQ ID NO: 78, and a second light chain constant region comprising the amino acid sequence of SEQ ID NO: 89; or
(g7) a first heavy chain constant region comprising the amino acid sequence of SEQ ID NO: 75, a first light chain constant region comprising the amino acid sequence of SEQ ID NO: 72, a second heavy chain constant region comprising the amino acid sequence of SEQ ID NO: 76, and a second light chain constant region comprising the amino acid sequence of SEQ ID NO: 89.

9. A multispecific antigen-binding molecule comprising four polypeptide chains, wherein the four polypeptide chains are any one of (h01) to (h18) below:
(h01) a heavy chain comprising the amino acid sequence of SEQ ID NO: 41 (chain 1) and a light chain comprising the amino acid sequence of SEQ ID NO: 50 (chain 2), and a heavy chain comprising the amino acid sequence of SEQ ID NO: 54 (chain 3) and a light chain comprising the amino acid sequence of SEQ ID NO: 68 (chain 4);
(h02) a heavy chain comprising the amino acid sequence of SEQ ID NO: 41 (chain 1) and a light chain comprising the amino acid sequence of SEQ ID NO: 50 (chain 2), and a heavy chain comprising the amino acid sequence of SEQ ID NO: 55 (chain 3) and a light chain comprising the amino acid sequence of SEQ ID NO: 68 (chain 4);
(h03) a heavy chain comprising the amino acid sequence of SEQ ID NO: 42 (chain 1) and a light chain comprising the amino acid sequence of SEQ ID NO: 51 (chain 2), and a heavy chain comprising the amino acid sequence of SEQ ID NO: 56 (chain 3) and a light chain comprising the amino acid sequence of SEQ ID NO: 69 (chain 4);
(h04) a heavy chain comprising the amino acid sequence of SEQ ID NO: 42 (chain 1) and a light chain comprising the amino acid sequence of SEQ ID NO: 51 (chain 2), and a heavy chain comprising the amino acid sequence of SEQ ID NO: 57 (chain 3) and a light chain comprising the amino acid sequence of SEQ ID NO: 69 (chain 4);
(h05) a heavy chain comprising the amino acid sequence of SEQ ID NO: 44 (chain 1) and a light chain comprising the amino acid sequence of SEQ ID NO: 52 (chain 2), and a heavy chain comprising the amino acid sequence of SEQ ID NO: 60 (chain 3) and a light chain comprising the amino acid sequence of SEQ ID NO: 70 (chain 4);
(h06) a heavy chain comprising the amino acid sequence of SEQ ID NO: 44 (chain 1) and a light chain comprising the amino acid sequence of SEQ ID NO: 52 (chain 2), and a heavy chain comprising the amino acid sequence of SEQ ID NO: 61 (chain 3) and a light chain comprising the amino acid sequence of SEQ ID NO: 70 (chain 4);
(h07) a heavy chain comprising the amino acid sequence of SEQ ID NO: 45 (chain 1) and a light chain comprising the amino acid sequence of SEQ ID NO: 50 (chain 2), and a heavy chain comprising the amino acid sequence of SEQ ID NO: 62 (chain 3) and a light chain comprising the amino acid sequence of SEQ ID NO: 68 (chain 4);
(h08) a heavy chain comprising the amino acid sequence of SEQ ID NO: 45 (chain 1) and a light chain comprising the amino acid sequence of SEQ ID NO: 50 (chain 2), and a heavy chain comprising the amino acid sequence of SEQ ID NO: 63 (chain 3) and a light chain comprising the amino acid sequence of SEQ ID NO: 68 (chain 4);
(h09) a heavy chain comprising the amino acid sequence of SEQ ID NO: 46 (chain 1) and a light chain comprising the amino acid sequence of SEQ ID NO: 51 (chain 2), and a heavy chain comprising the amino acid sequence of SEQ ID NO: 64 (chain 3) and a light chain comprising the amino acid sequence of SEQ ID NO: 69 (chain 4);
(h10) a heavy chain comprising the amino acid sequence of SEQ ID NO: 46 (chain 1) and a light chain comprising the amino acid sequence of SEQ ID NO: 51 (chain 2), and a heavy chain comprising the amino acid sequence of SEQ ID NO: 65 (chain 3) and a light chain comprising the amino acid sequence of SEQ ID NO: 69 (chain 4);
(h11) a heavy chain comprising the amino acid sequence of SEQ ID NO: 47 (chain 1) and a light chain comprising the amino acid sequence of SEQ ID NO: 52 (chain 2), and a heavy chain comprising the amino acid sequence of SEQ ID NO: 66 (chain 3) and a light chain comprising the amino acid sequence of SEQ ID NO: 70 (chain 4);
(h12) a heavy chain comprising the amino acid sequence of SEQ ID NO: 47 (chain 1) and a light chain comprising the amino acid sequence of SEQ ID NO: 52 (chain 2), and a heavy chain comprising the amino acid sequence of SEQ ID NO: 67 (chain 3) and a light chain comprising the amino acid sequence of SEQ ID NO: 70 (chain 4);
(h13) a heavy chain comprising the amino acid sequence of SEQ ID NO: 48 (chain 1) and a light chain comprising the amino acid sequence of SEQ ID NO: 53 (chain 2), and a heavy chain comprising the amino acid sequence of SEQ ID NO: 56 (chain 3) and a light chain comprising the amino acid sequence of SEQ ID NO: 71 (chain 4);
(h14) a heavy chain comprising the amino acid sequence of SEQ ID NO: 48 (chain 1) and a light chain comprising the amino acid sequence of SEQ ID NO: 53 (chain 2), and a heavy chain comprising the amino acid sequence of SEQ ID NO: 57 (chain 3) and a light chain comprising the amino acid sequence of SEQ ID NO: 71 (chain 4);
(h15) a heavy chain comprising the amino acid sequence of SEQ ID NO: 49 (chain 1) and a light chain comprising the amino acid sequence of SEQ ID NO: 53 (chain 2), and a heavy chain comprising the amino acid sequence of SEQ ID NO: 64 (chain 3) and a light chain comprising the amino acid sequence of SEQ ID NO: 71 (chain 4);
(h16) a heavy chain comprising the amino acid sequence of SEQ ID NO: 49 (chain 1) and a light chain comprising the amino acid sequence of SEQ ID NO: 53 (chain 2), and a heavy chain comprising the amino acid sequence of SEQ ID NO: 65 (chain 3) and a light chain comprising the amino acid sequence of SEQ ID NO: 71 (chain 4);
(h17) a heavy chain comprising the amino acid sequence of SEQ ID NO: 43 (chain 1) and a light chain comprising the amino acid sequence of SEQ ID NO: 52 (chain 2), and a heavy chain comprising the amino acid sequence of SEQ ID NO: 58 (chain 3) and a light chain comprising the amino acid sequence of SEQ ID NO: 70 (chain 4); and
(h18) a heavy chain comprising the amino acid sequence of SEQ ID NO: 43 (chain 1) and a light chain comprising the amino acid sequence of SEQ ID NO: 52 (chain 2), and a heavy chain comprising the amino acid sequence of SEQ ID NO: 59 (chain 3) and a light chain comprising the amino acid sequence of SEQ ID NO: 70 (chain 4).

10. An isolated nucleic acid encoding the multispecific antigen-binding molecule of claim 2.

11. A host cell comprising the nucleic acid of claim 10.

12. A method of producing a multispecific antigen-binding molecule comprising culturing the host cell of claim 11 so that the multispecific antigen-binding molecule is produced.

13. A pharmaceutical composition comprising the multispecific antigen-binding molecule of claim 2, and a pharmaceutically acceptable carrier.

14. A method of treating an individual having cancer comprising administering to the individual an effective amount of the multispecific antigen-binding molecule of claim 2.

15. The method of claim 14, wherein the cancer comprises CLDN6 expressing cells.

16. The multispecific antigen-binding molecule of claim 5, which further comprises:
(iii) an Fc domain which exhibits reduced binding affinity to human Fc gamma receptor, as compared to a native human IgG1 Fc domain.

17. The multispecific antigen-binding molecule of claim 5, wherein the first antibody variable region of the first antigen-binding moiety is fused with a first heavy chain constant region, the second antibody variable region of the first antigen-binding moiety is fused with a first light chain constant region, the third antibody variable region of the second antigen-binding moiety is fused with a second heavy chain constant region, the fourth antibody variable region of the second antigen-binding moiety is fused with a second light chain constant region, wherein the constant regions comprises:
(g1) a first heavy chain constant region comprising the amino acid sequence of SEQ ID NO: 74, a first light chain constant region comprising the amino acid sequence of SEQ ID NO: 87, a second heavy chain constant region comprising the amino acid sequence of SEQ ID NO: 73, and a second light chain constant region comprising the amino acid sequence of SEQ ID NO: 88;
(g2) a first heavy chain constant region comprising the amino acid sequence of SEQ ID NO: 74, a first light chain constant region comprising the amino acid sequence of SEQ ID NO: 85, a second heavy chain constant region comprising the amino acid sequence of SEQ ID NO: 81, and a second light chain constant region comprising the amino acid sequence of SEQ ID NO: 86;
(g3) a first heavy chain constant region comprising the amino acid sequence of SEQ ID NO: 79, a first light chain constant region comprising the amino acid sequence of SEQ ID NO: 72, a second heavy chain constant region comprising the amino acid sequence of SEQ ID NO: 80, and a second light chain constant region comprising the amino acid sequence of SEQ ID NO: 89;
(g4) a first heavy chain constant region comprising the amino acid sequence of SEQ ID NO: 83, a first light chain constant region comprising the amino acid sequence of SEQ ID NO: 87, a second heavy chain constant region comprising the amino acid sequence of SEQ ID NO: 82, and a second light chain constant region comprising the amino acid sequence of SEQ ID NO: 88;
(g5) a first heavy chain constant region comprising the amino acid sequence of SEQ ID NO: 83, a first light chain constant region comprising the amino acid sequence of SEQ ID NO: 85, a second heavy chain constant region comprising the amino acid sequence of SEQ ID NO: 84, and a second light chain constant region comprising the amino acid sequence of SEQ ID NO: 86;
(g6) a first heavy chain constant region comprising the amino acid sequence of SEQ ID NO: 77, a first light chain constant region comprising the amino acid sequence of SEQ ID NO: 72, a second heavy chain constant region comprising the amino acid sequence of SEQ ID NO: 78, and a second light chain constant region comprising the amino acid sequence of SEQ ID NO: 89; or
(g7) a first heavy chain constant region comprising the amino acid sequence of SEQ ID NO: 75, a first light chain constant region comprising the amino acid sequence of SEQ ID NO: 72, a second heavy chain constant region comprising the amino acid sequence of SEQ ID NO: 76, and a second light chain constant region comprising the amino acid sequence of SEQ ID NO: 89.

18. An isolated nucleic acid encoding the multispecific antigen-binding molecule of claim 5.

19. A host cell comprising the nucleic acid of claim 18.

20. A method of producing a multispecific antigen-binding molecule comprising culturing the host cell of claim 19 so that the multispecific antigen-binding molecule is produced.

21. A pharmaceutical composition comprising the multispecific antigen-binding molecule of claim 5 and a pharmaceutically acceptable carrier.

22. A method of treating an individual having cancer comprising administering to the individual an effective amount of the multispecific antigen-binding molecule of claim 5.

23. The method of claim 22, wherein the cancer comprises CLDN6 expressing cells.

24. The method of claim 23, wherein the cancer is ovarian cancer, non-small cell lung cancer, gastric cancer, liver cancer, endometrial cancer, or germ cell tumor.

25. The method of claim 14, wherein the cancer is ovarian cancer, non-small cell lung cancer, gastric cancer, liver cancer, endometrial cancer, or germ cell tumor.

26. A kit comprising the multispecific antigen-binding molecule of claim 2 and instructions for use.

27. A kit comprising the multispecific antigen-binding molecule of claim 5 and instructions for use.

28. A kit comprising the pharmaceutical composition of claim 13 and instructions for use.
